SEQUENCE LISTING (251 sequences)

SEQ ID NO: 1 - DNA, 681 bp, synthetic construct
  source 1..681: mol_type = other DNA
atgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctactttcacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatag

SEQ ID NO: 2 - DNA, 786 bp, synthetic construct
  source 1..786: mol_type = other DNA
atgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 3 - DNA, 417 bp, synthetic construct
  source 1..417: mol_type = other DNA
atggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataa

SEQ ID NO: 4 - DNA, 1422 bp, synthetic construct
  source 1..1422: mol_type = other DNA
atgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatataaagttcctgagttcgattcgtccacaattaaaaatatctcttctgcaaaaggcctggacgtttgggacagctggccattacaaaacactgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataa

SEQ ID NO: 5 - DNA, 375 bp, synthetic construct
  source 1..375: mol_type = other DNA
atggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaa

SEQ ID NO: 6 - DNA, 984 bp, synthetic construct
  source 1..984: mol_type = other DNA
atgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttgccgaaccttctgcagaagtggacgtcatgggtaaaaacggtaaatggctggaagtgctgggctgcgggatggtgcatccgaacgtgttgcgtaacgttggcatcgacccggaagtttactctggtttcggcttcgggatggggatggagcgtctgactatgttgcgttacggcgtcaccgacctgcgttcattcttcgaaaacgatctgcgtttcctcaaacagtttaaataa

SEQ ID NO: 7 - DNA, 660 bp, synthetic construct
  source 1..660: mol_type = other DNA
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccgtgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 8 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 9 - DNA, 30 bp, synthetic construct
  source 1..30: mol_type = other DNA
gatgcaagtgtgtcgctgtcgacaggccct

SEQ ID NO: 10 - DNA, 4107 bp, synthetic construct
  source 1..4107: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 11 - DNA, 461 bp, synthetic construct
  source 1..461: mol_type = other DNA
aaaaagtttaaattaaatccataatgatttgatgatttcaataatagttttaatgacctccgaaattagtttaatatgctttaatttttctttttcaaaatatctcttcaaaaaatattacccaatacttaataataaatagattataacacaaaattcttttaaaaagtagtttattttgttatcattctatagtattaagtattgttttatggctgataaatttctttgaatttctccttgattatttgttataaaagttataaaataatcttgttggaaccattcaaaacagcatagcaagttaaaataaggctagtccgttatcaacttgaaaaagtggcaccgagtcggtgctttttttgatacttctattctactctgactgcaaaccaaaaaaacaagcgctttcaaaacgcttgttttatcatttttagggaaattaatctcttaatcctttt

SEQ ID NO: 12 - DNA, 30 bp, synthetic construct
  source 1..30: mol_type = other DNA
cggccgccctttagtgagggtaagcttcct

SEQ ID NO: 13 - DNA, 30 bp, synthetic construct
  source 1..30: mol_type = other DNA
attaattgtcaacacgtgctcaagcttcct

SEQ ID NO: 14 - DNA, 30 bp, synthetic construct
  source 1..30: mol_type = other DNA
ccggattgcgcgtaatcgtcaccatcccct

SEQ ID NO: 15 - DNA, 30 bp, synthetic construct
  source 1..30: mol_type = other DNA
ttcagcaaggagctgtgaaaatgtctccct

SEQ ID NO: 16 - DNA, 30 bp, synthetic construct
  source 1..30: mol_type = other DNA
cattttcaacattgttgcagctggcagcct

SEQ ID NO: 17 - DNA, 30 bp, synthetic construct
  source 1..30: mol_type = other DNA
ggatatctggggcatgacatggaagaccct

SEQ ID NO: 18 - DNA, 30 bp, synthetic construct
  source 1..30: mol_type = other DNA
tgattgtaatgcttagccattatcttccct

SEQ ID NO: 19 - DNA, 30 bp, synthetic construct
  source 1..30: mol_type = other DNA
aaccaggaaacagaacctctgacaatgcct

SEQ ID NO: 20 - DNA, 30 bp, synthetic construct
  source 1..30: mol_type = other DNA
gacgaaacctaacaggaagcacatcaccct

SEQ ID NO: 21 - DNA, 30 bp, synthetic construct
  source 1..30: mol_type = other DNA
ccttgttacctgatcagcgtaaacacccct

SEQ ID NO: 22 - DNA, 30 bp, synthetic construct
  source 1..30: mol_type = other DNA
tgtgccaggcgcaggttgaccatgctgcct

SEQ ID NO: 23 - DNA, 30 bp, synthetic construct
  source 1..30: mol_type = other DNA
catcttatccagcaaccaggtcgcatccct

SEQ ID NO: 24 - DNA, 30 bp, synthetic construct
  source 1..30: mol_type = other DNA
gacatttaggtgacactatagaaggatccg

SEQ ID NO: 25 - DNA, 30 bp, synthetic construct
  source 1..30: mol_type = other DNA
cggatccttctatagtgtcacctaaatgtc

SEQ ID NO: 26 - DNA, 13 bp, synthetic construct
  source 1..13: mol_type = other DNA
gacaggccctagg

SEQ ID NO: 27 - DNA, 13 bp, synthetic construct
  source 1..13: mol_type = other DNA
cctaggaagctta

SEQ ID NO: 28 - DNA, 26 bp, synthetic construct
  source 1..26: mol_type = other DNA
caggagcccatcaaaggtgaacatgg

SEQ ID NO: 29 - DNA, 26 bp, synthetic construct
  source 1..26: mol_type = other DNA
ctgaccccaaacctagagtcagcatc

SEQ ID NO: 30 - RNA, 10 nt, synthetic construct
  source 1..10: mol_type = other RNA
gacaggccct

SEQ ID NO: 31 - RNA, 10 nt, synthetic construct
  source 1..10: mol_type = other RNA
taagcttcct

SEQ ID NO: 32 - DNA, 24 bp, synthetic construct
  source 1..24: mol_type = other DNA
ccgtaatattgccggatggtgacg

SEQ ID NO: 33 - DNA, 24 bp, synthetic construct
  source 1..24: mol_type = other DNA
gaaaatgtctcaaaataaggcttt

SEQ ID NO: 34 - DNA, 24 bp, synthetic construct
  source 1..24: mol_type = other DNA
ggtaaaagctttcctgccagctgc

SEQ ID NO: 35 - DNA, 24 bp, synthetic construct
  source 1..24: mol_type = other DNA
acatggaagacgactgcgacatta

SEQ ID NO: 36 - DNA, 24 bp, synthetic construct
  source 1..24: mol_type = other DNA
aaacgataattcagaagataatgg

SEQ ID NO: 37 - DNA, 24 bp, synthetic construct
  source 1..24: mol_type = other DNA
ctctgacaatgaatttacagcatc

SEQ ID NO: 38 - DNA, 24 bp, synthetic construct
  source 1..24: mol_type = other DNA
cgctgtcgtctttgtgatgtgctt

SEQ ID NO: 39 - DNA, 24 bp, synthetic construct
  source 1..24: mol_type = other DNA
gcgtaaacaccttatctggcctac

SEQ ID NO: 40 - DNA, 24 bp, synthetic construct
  source 1..24: mol_type = other DNA
cttacatcgctaccagcatggtca

SEQ ID NO: 41 - DNA, 24 bp, synthetic construct
  source 1..24: mol_type = other DNA
caggtcgcatccggcaagatcacc

SEQ ID NO: 42 - DNA, 30 bp, synthetic construct
  source 1..30: mol_type = other DNA
acaatttggtaaaggctccatcatgcgcct

SEQ ID NO: 43 - DNA, 30 bp, synthetic construct
  source 1..30: mol_type = other DNA
ttgatagcaaagggattttccgccaccagt

SEQ ID NO: 44 - DNA, 30 bp, synthetic construct
  source 1..30: mol_type = other DNA
ctggttttcttccagccagcgcagagccgc

SEQ ID NO: 45 - DNA, 30 bp, synthetic construct
  source 1..30: mol_type = other DNA
cacgcccgcatagatgagtttcagacgaga

SEQ ID NO: 46 - DNA, 30 bp, synthetic construct
  source 1..30: mol_type = other DNA
cgcgaagctgaacatcaggcttttcttgac

SEQ ID NO: 47 - DNA, 30 bp, synthetic construct
  source 1..30: mol_type = other DNA
acgccagagaatttaaaacgcgataaaggc

SEQ ID NO: 48 - DNA, 30 bp, synthetic construct
  source 1..30: mol_type = other DNA
agaggcttcaatcaggcgctcaccctgtaa

SEQ ID NO: 49 - DNA, 30 bp, synthetic construct
  source 1..30: mol_type = other DNA
cagcgcgcatttgtcctgcatagtcgcccg

SEQ ID NO: 50 - DNA, 30 bp, synthetic construct
  source 1..30: mol_type = other DNA
gcaaaacaacaaattacccaaagcgagcac

SEQ ID NO: 51 - DNA, 30 bp, synthetic construct
  source 1..30: mol_type = other DNA
catgccagtgatgatgagttgtttgccagt

SEQ ID NO: 52 - DNA, 30 bp, synthetic construct
  source 1..30: mol_type = other DNA
gaagtgaaaggtgaagcgtaccgtgcattc

SEQ ID NO: 53 - DNA, 30 bp, synthetic construct
  source 1..30: mol_type = other DNA
aagacgcgcgtactggtgcatcgtatcgcc

SEQ ID NO: 54 - DNA, 30 bp, synthetic construct
  source 1..30: mol_type = other DNA
gtccgcaatcatctgccgctgcggcgcacg

SEQ ID NO: 55 - DNA, 30 bp, synthetic construct
  source 1..30: mol_type = other DNA
cccggcattgaggatcaccgcccgatcgta

SEQ ID NO: 56 - DNA, 30 bp, synthetic construct
  source 1..30: mol_type = other DNA
gaaatgtcagaatatctgtttgataagctt

SEQ ID NO: 57 - DNA, 30 bp, synthetic construct
  source 1..30: mol_type = other DNA
atcacccatccggtaaaacagcaggatctc

SEQ ID NO: 58 - DNA, 30 bp, synthetic construct
  source 1..30: mol_type = other DNA
tgtaatgaatgcggggccgattatccgcgc

SEQ ID NO: 59 - DNA, 30 bp, synthetic construct
  source 1..30: mol_type = other DNA
gaagccgggcatttacattctatgatcctc

SEQ ID NO: 60 - DNA, 30 bp, synthetic construct
  source 1..30: mol_type = other DNA
attctggtgcaaagtaccggtatggcacag

SEQ ID NO: 61 - DNA, 30 bp, synthetic construct
  source 1..30: mol_type = other DNA
tgtgtcagtgaaatcggtgagctacaaaat

SEQ ID NO: 62 - DNA, 114 bp, synthetic construct
  source 1..114: mol_type = other DNA
gacaaatagtgcgattacgaaattttttagacaaaaatagtctacgaggttttagagctatgctgttttgaatggtcccaaaacgatgcaagtgtgtcgctgtcgacaggccct

SEQ ID NO: 63 - DNA, 114 bp, synthetic construct
  source 1..114: mol_type = other DNA
gacaaatagtgcgattacgaaattttttagacaaaaatagtctacgaggttttagagctatgctgttttgaatggtcccaaaacccggattgcgcgtaatcgtcaccatcccct

SEQ ID NO: 64 - DNA, 114 bp, synthetic construct
  source 1..114: mol_type = other DNA
gacaaatagtgcgattacgaaattttttagacaaaaatagtctacgaggttttagagctatgctgttttgaatggtcccaaaaccattttcaacattgttgcagctggcagcct

SEQ ID NO: 65 - DNA, 114 bp, synthetic construct
  source 1..114: mol_type = other DNA
gacaaatagtgcgattacgaaattttttagacaaaaatagtctacgaggttttagagctatgctgttttgaatggtcccaaaactgattgtaatgcttagccattatcttccct

SEQ ID NO: 66 - DNA, 114 bp, synthetic construct
  source 1..114: mol_type = other DNA
gacaaatagtgcgattacgaaattttttagacaaaaatagtctacgaggttttagagctatgctgttttgaatggtcccaaaacgacgaaacctaacaggaagcacatcaccct

SEQ ID NO: 67 - DNA, 114 bp, synthetic construct
  source 1..114: mol_type = other DNA
gacaaatagtgcgattacgaaattttttagacaaaaatagtctacgaggttttagagctatgctgttttgaatggtcccaaaactgtgccaggcgcaggttgaccatgctgcct

SEQ ID NO: 68 - DNA, 96 bp, synthetic construct
  source 1..96: mol_type = other DNA
gatgcaagtgtgtcgctgtcgacaggccctgttttagagctatgctgttttgaatggtcccaaaaccggccgccctttagtgagggtaagcttcct

SEQ ID NO: 69 - DNA, 96 bp, synthetic construct
  source 1..96: mol_type = other DNA
gatgcaagtgtgtcgctgtcgacaggccctgttttagagctatgctgttttgaatggtcccaaaacattaattgtcaacacgtgctcaagcttcct

SEQ ID NO: 70 - DNA, 96 bp, synthetic construct
  source 1..96: mol_type = other DNA
ccggattgcgcgtaatcgtcaccatcccctgttttagagctatgctgttttgaatggtcccaaaacttcagcaaggagctgtgaaaatgtctccct

SEQ ID NO: 71 - DNA, 96 bp, synthetic construct
  source 1..96: mol_type = other DNA
cattttcaacattgttgcagctggcagcctgttttagagctatgctgttttgaatggtcccaaaacggatatctggggcatgacatggaagaccct

SEQ ID NO: 72 - DNA, 96 bp, synthetic construct
  source 1..96: mol_type = other DNA
tgattgtaatgcttagccattatcttccctgttttagagctatgctgttttgaatggtcccaaaacaaccaggaaacagaacctctgacaatgcct

SEQ ID NO: 73 - DNA, 96 bp, synthetic construct
  source 1..96: mol_type = other DNA
gacgaaacctaacaggaagcacatcaccctgttttagagctatgctgttttgaatggtcccaaaacccttgttacctgatcagcgtaaacacccct

SEQ ID NO: 74 - DNA, 96 bp, synthetic construct
  source 1..96: mol_type = other DNA
tgtgccaggcgcaggttgaccatgctgcctgttttagagctatgctgttttgaatggtcccaaaaccatcttatccagcaaccaggtcgcatccct

SEQ ID NO: 75 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacaggaagcttaccctcactaaagggcggccg

SEQ ID NO: 76 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacaggaagcttgagcacgtgttgacaattaat

SEQ ID NO: 77 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacagggagacattttcacagctccttgctgaa

SEQ ID NO: 78 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacagggtcttccatgtcatgccccagatatcc

SEQ ID NO: 79 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacaggcattgtcagaggttctgtttcctggtt

SEQ ID NO: 80 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacaggggtgtttacgctgatcaggtaacaagg

SEQ ID NO: 81 - DNA, 116 bp, synthetic construct
  source 1..116: mol_type = other DNA
cagcaggacgcactgaccgaattcaactcaacaagtctcagtgtgctgaagttttgggaccattcaaaacagcatagctctaaaacagggatgcgacctggttgctggataagatg

SEQ ID NO: 82 - DNA, 26 bp, synthetic construct
  source 1..26: mol_type = other DNA
gtcgccagacagaatgcgaagaattg

SEQ ID NO: 83 - DNA, 27 bp, synthetic construct
  source 1..27: mol_type = other DNA
cgctccactcattcgtaataccgttcg

SEQ ID NO: 84 - DNA, 28 bp, synthetic construct
  source 1..28: mol_type = other DNA
gctttactggttagcggatgtcagattg

SEQ ID NO: 85 - DNA, 25 bp, synthetic construct
  source 1..25: mol_type = other DNA
gggcaaacggtgtggattaaagacg

SEQ ID NO: 86 - DNA, 25 bp, synthetic construct
  source 1..25: mol_type = other DNA
tggcatcacgagacacatcacagag

SEQ ID NO: 87 - DNA, 27 bp, synthetic construct
  source 1..27: mol_type = other DNA
ttaccgcctggttctctgacttaacgc

SEQ ID NO: 88 - DNA, 27 bp, synthetic construct
  source 1..27: mol_type = other DNA
catgtgggcgttcagctttattcttcg

SEQ ID NO: 89 - DNA, 31 bp, synthetic construct
  source 1..31: mol_type = other DNA
ctttccactaatgcaggctacccaatcactg

SEQ ID NO: 90 - DNA, 30 bp, synthetic construct
  source 1..30: mol_type = other DNA
gtgttccgtccaatctccaacaacgatctg

SEQ ID NO: 91 - DNA, 24 bp, synthetic construct
  source 1..24: mol_type = other DNA
tgcattacgacaccaccctgaacg

SEQ ID NO: 92 - DNA, 27 bp, synthetic construct
  source 1..27: mol_type = other DNA
tgagactcacaaatccacgatgacagg

SEQ ID NO: 93 - DNA, 28 bp, synthetic construct
  source 1..28: mol_type = other DNA
ggcaagcgggaataccaaagaagaatag

SEQ ID NO: 94 - DNA, 30 bp, synthetic construct
  source 1..30: mol_type = other DNA
cccgtcagcaggagaatcagaagtaagaac

SEQ ID NO: 95 - DNA, 29 bp, synthetic construct
  source 1..29: mol_type = other DNA
atcgtcaccacaaataacgccacagactc

SEQ ID NO: 96 - DNA, 24 bp, synthetic construct
  source 1..24: mol_type = other DNA
tacaacatgaccagcggattcaag

SEQ ID NO: 97 - DNA, 28 bp, synthetic construct
  source 1..28: mol_type = other DNA
ttcttgttccagttgacctttctcccag

SEQ ID NO: 98 - DNA, 25 bp, synthetic construct
  source 1..25: mol_type = other DNA
gtgcgatagacaaagcagtcctgag

SEQ ID NO: 99 - DNA, 28 bp, synthetic construct
  source 1..28: mol_type = other DNA
cagaaccaatgtctttcaccattccacg

SEQ ID NO: 100 - DNA, 26 bp, synthetic construct
  source 1..26: mol_type = other DNA
agaggtcaacattacagccagcagag

SEQ ID NO: 101 - DNA, 23 bp, synthetic construct
  source 1..23: mol_type = other DNA
aaacgcagccactgaggcaaatg

SEQ ID NO: 102 - DNA, 49 bp, synthetic construct
  source 1..49: mol_type = other DNA
cgcggaacccctatttgtttatttttctaaatacattcaaatatgtatc

SEQ ID NO: 103 - DNA, 49 bp, synthetic construct
  source 1..49: mol_type = other DNA
ggtgcctcactgattaagcattggtaaagcgcttttccgctgcataacc

SEQ ID NO: 104 - DNA, 60 bp, synthetic construct
  source 1..60: mol_type = other DNA
ggatcctatttcttaataactaaaaatatggtataatactcttaataaatgcagtaatac

SEQ ID NO: 105 - DNA, 49 bp, synthetic construct
  source 1..49: mol_type = other DNA
gtttaaataaggaggacaatccgcggaacccctatttgtttatttttct

SEQ ID NO: 106 - DNA, 60 bp, synthetic construct
  source 1..60: mol_type = other DNA
cattcttcgaaaacgatctgcgtttcctcaaacagtttaaataaggaggacaatccgcgg

SEQ ID NO: 107 - DNA, 33 bp, synthetic construct
  source 1..33: mol_type = other DNA
ttaccaatgcttaatcagtgaggcacctatctc

SEQ ID NO: 108 - DNA, 24 bp, synthetic construct
  source 1..24: mol_type = other DNA
tcatctgttacgccggcggtagcc

SEQ ID NO: 109 - DNA, 27 bp, synthetic construct
  source 1..27: mol_type = other DNA
gatactgagcacatcagcaggacgcac

SEQ ID NO: 110 - DNA, 51 bp, synthetic construct
  source 1..51: mol_type = other DNA
tttttagttattaagaaataggatcctcatctgttacgccggcggtagccg

SEQ ID NO: 111 - DNA, 81 bp, synthetic construct
  source 1..81: mol_type = other DNA
ccttagctttcgctaaggatgataagcgtagcgcatcaggcaatttagcgtttggatactgagcacatcagcaggacgcac

SEQ ID NO: 112 - DNA, 49 bp, synthetic construct
  source 1..49: mol_type = other DNA
gtgaagcgtcctaaggcttaacgcggaacccctatttgtttatttttct

SEQ ID NO: 113 - DNA, 60 bp, synthetic construct
  source 1..60: mol_type = other DNA
gttaaagaccgtaagcaggctcgttccaagtatggcgtgaagcgtcctaaggcttaacgc

SEQ ID NO: 114 - DNA, 46 bp, synthetic construct
  source 1..46: mol_type = other DNA
aatggcgatagagtatattttagatgaagatgctaggatccctagg

SEQ ID NO: 115 - DNA, 41 bp, synthetic construct
  source 1..41: mol_type = other DNA
taaaatatactctatcgccattgctccccaaatacaaaacc

SEQ ID NO: 116 - DNA, 34 bp, synthetic construct
  source 1..34: mol_type = other DNA
aacacaatttggtaaaggctccatcatgcgcctg

SEQ ID NO: 117 - DNA, 34 bp, synthetic construct
  source 1..34: mol_type = other DNA
aacttgatagcaaagggattttccgccaccagtg

SEQ ID NO: 118 - DNA, 34 bp, synthetic construct
  source 1..34: mol_type = other DNA
aacctggttttcttccagccagcgcagagccgcg

SEQ ID NO: 119 - DNA, 34 bp, synthetic construct
  source 1..34: mol_type = other DNA
aaccacgcccgcatagatgagtttcagacgagag

SEQ ID NO: 120 - DNA, 34 bp, synthetic construct
  source 1..34: mol_type = other DNA
aaccgcgaagctgaacatcaggcttttcttgacg

SEQ ID NO: 121 - DNA, 34 bp, synthetic construct
  source 1..34: mol_type = other DNA
aacacgccagagaatttaaaacgcgataaaggcg

SEQ ID NO: 122 - DNA, 34 bp, synthetic construct
  source 1..34: mol_type = other DNA
aacagaggcttcaatcaggcgctcaccctgtaag

SEQ ID NO: 123 - DNA, 34 bp, synthetic construct
  source 1..34: mol_type = other DNA
aaccagcgcgcatttgtcctgcatagtcgcccgg

SEQ ID NO: 124 - DNA, 34 bp, synthetic construct
  source 1..34: mol_type = other DNA
aacgcaaaacaacaaattacccaaagcgagcacg

SEQ ID NO: 125 - DNA, 34 bp, synthetic construct
  source 1..34: mol_type = other DNA
aaaccaagaggtcggaataatattgccgcaggtc

SEQ ID NO: 126 - DNA, 34 bp, synthetic construct
  source 1..34: mol_type = other DNA
aacgaagtgaaaggtgaagcgtaccgtgcattcg

SEQ ID NO: 127 - DNA, 34 bp, synthetic construct
  source 1..34: mol_type = other DNA
aacaagacgcgcgtactggtgcatcgtatcgccg

SEQ ID NO: 128 - DNA, 34 bp, synthetic construct
  source 1..34: mol_type = other DNA
aacgtccgcaatcatctgccgctgcggcgcacgg

SEQ ID NO: 129 - DNA, 34 bp, synthetic construct
  source 1..34: mol_type = other DNA
aaccccggcattgaggatcaccgcccgatcgtag

SEQ ID NO: 130 - DNA, 34 bp, synthetic construct
  source 1..34: mol_type = other DNA
aacgaaatgtcagaatatctgtttgataagcttg

SEQ ID NO: 131 - DNA, 34 bp, synthetic construct
  source 1..34: mol_type = other DNA
aacatcacccatccggtaaaacagcaggatctcg

SEQ ID NO: 132 - DNA, 34 bp, synthetic construct
  source 1..34: mol_type = other DNA
aactgtaatgaatgcggggccgattatccgcgcg

SEQ ID NO: 133 - DNA, 34 bp, synthetic construct
  source 1..34: mol_type = other DNA
aacatttcgggtcttgggggtagcggcagttacg

SEQ ID NO: 134 - DNA, 34 bp, synthetic construct
  source 1..34: mol_type = other DNA
aacattctggtgcaaagtaccggtatggcacagg

SEQ ID NO: 135 - DNA, 34 bp, synthetic construct
  source 1..34: mol_type = other DNA
aactgtgtcagtgaaatcggtgagctacaaaatg

SEQ ID NO: 136 - DNA, 34 bp, synthetic construct
  source 1..34: mol_type = other DNA
aaacaggcgcatgatggagcctttaccaaattgt

SEQ ID NO: 137 - DNA, 34 bp, synthetic construct
  source 1..34: mol_type = other DNA
aaacactggtggcggaaaatccctttgctatcaa

SEQ ID NO: 138 - DNA, 34 bp, synthetic construct
  source 1..34: mol_type = other DNA
aaacgcggctctgcgctggctggaagaaaaccag

SEQ ID NO: 139 - DNA, 34 bp, synthetic construct
  source 1..34: mol_type = other DNA
aaactctcgtctgaaactcatctatgcgggcgtg

SEQ ID NO: 140 - DNA, 34 bp, synthetic construct
  source 1..34: mol_type = other DNA
aaacgtcaagaaaagcctgatgttcagcttcgcg

SEQ ID NO: 141 - DNA, 34 bp, synthetic construct
  source 1..34: mol_type = other DNA
aaacgcctttatcgcgttttaaattctctggcgt

SEQ ID NO: 142 - DNA, 34 bp, synthetic construct
  source 1..34: mol_type = other DNA
aaacttacagggtgagcgcctgattgaagcctct

SEQ ID NO: 143 - DNA, 34 bp, synthetic construct
  source 1..34: mol_type = other DNA
aaaccgggcgactatgcaggacaaatgcgcgctg

SEQ ID NO: 144 - DNA, 34 bp, synthetic construct
  source 1..34: mol_type = other DNA
aaacgtgctcgctttgggtaatttgttgttttgc

SEQ ID NO: 145 - DNA, 34 bp, synthetic construct
  source 1..34: mol_type = other DNA
aacgacctgcggcaatattattccgacctcttgg

SEQ ID NO: 146 - DNA, 34 bp, synthetic construct
  source 1..34: mol_type = other DNA
aaacgaatgcacggtacgcttcacctttcacttc

SEQ ID NO: 147 - DNA, 34 bp, synthetic construct
  source 1..34: mol_type = other DNA
aaacggcgatacgatgcaccagtacgcgcgtctt

SEQ ID NO: 148 - DNA, 34 bp, synthetic construct
  source 1..34: mol_type = other DNA
aaaccgtgcgccgcagcggcagatgattgcggac

SEQ ID NO: 149 - DNA, 34 bp, synthetic construct
  source 1..34: mol_type = other DNA
aaactacgatcgggcggtgatcctcaatgccggg

SEQ ID NO: 150 - DNA, 34 bp, synthetic construct
  source 1..34: mol_type = other DNA
aaacaagcttatcaaacagatattctgacatttc

SEQ ID NO: 151 - DNA, 34 bp, synthetic construct
  source 1..34: mol_type = other DNA
aaacgagatcctgctgttttaccggatgggtgat

SEQ ID NO: 152 - DNA, 34 bp, synthetic construct
  source 1..34: mol_type = other DNA
aaacgcgcggataatcggccccgcattcattaca

SEQ ID NO: 153 - DNA, 34 bp, synthetic construct
  source 1..34: mol_type = other DNA
aaacgtaactgccgctacccccaagacccgaaat

SEQ ID NO: 154 - DNA, 34 bp, synthetic construct
  source 1..34: mol_type = other DNA
aaacctgtgccataccggtactttgcaccagaat

SEQ ID NO: 155 - DNA, 34 bp, synthetic construct
  source 1..34: mol_type = other DNA
aaacattttgtagctcaccgatttcactgacaca

SEQ ID NO: 156 - DNA, 70 bp, synthetic construct
  source 1..70: mol_type = other DNA
caatttggtaaaggctccatcatgcgcctgtaataagcttgaagaccgttccatggatgtggaaaccatc

SEQ ID NO: 157 - DNA, 70 bp, synthetic construct
  source 1..70: mol_type = other DNA
ctttccggccgcgattgcctcgtcgtcatgtaataagcttggcggaaaatccctttgctatcaaatccct

SEQ ID NO: 158 - DNA, 70 bp, synthetic construct
  source 1..70: mol_type = other DNA
ctgtgcgcccgtttttctctgaaagatacgtaataagcttctgcgctggctggaagaaaaccagcttgaa

SEQ ID NO: 159 - DNA, 70 bp, synthetic construct
  source 1..70: mol_type = other DNA
ggatgcatccgcaccaaagtggatgatttataataagcttctgaaactcatctatgcgggcgtgacggaa

SEQ ID NO: 160 - DNA, 70 bp, synthetic construct
  source 1..70: mol_type = other DNA
cgcgaagctgaacatcaggcttttcttgactaataagcttgagacagcacaaacccatcaggtggatgcg

SEQ ID NO: 161 - DNA, 70 bp, synthetic construct
  source 1..70: mol_type = other DNA
acgccagagaatttaaaacgcgataaaggctaataagcttgtgttactggagatctggctaggtgccagc

SEQ ID NO: 162 - DNA, 73 bp, synthetic construct
  source 1..73: mol_type = other DNA
tgtcgccgagacactagatcctttgcgcttgtaataagcttggtgagcgcctgattgaagcctctgccggcac

SEQ ID NO: 163 - DNA, 70 bp, synthetic construct
  source 1..70: mol_type = other DNA
cagcgcgcatttgtcctgcatagtcgcccgtaataagcttaccagcctgatgctggacgtcttcacggag

SEQ ID NO: 164 - DNA, 70 bp, synthetic construct
  source 1..70: mol_type = other DNA
gcaaaacaacaaattacccaaagcgagcactaataagcttgagtgcgagttactcaataacgaagtgcgc

SEQ ID NO: 165 - DNA, 70 bp, synthetic construct
  source 1..70: mol_type = other DNA
ataaatataacccactccctcctgcgttttaagcttattacaagaggtcggaataatattgccgcaggtc

SEQ ID NO: 166 - DNA, 70 bp, synthetic construct
  source 1..70: mol_type = other DNA
gaagtgaaaggtgaagcgtaccgtgcattctaataagcttaatcgggcgcgtaaccaacccgacggtaat

SEQ ID NO: 167 - DNA, 70 bp, synthetic construct
  source 1..70: mol_type = other DNA
aagacgcgcgtactggtgcatcgtatcgcctaataagcttagcgtggaaaactgctcgccatactcgatt

SEQ ID NO: 168 - DNA, 70 bp, synthetic construct
  source 1..70: mol_type = other DNA
gcgcttcaggaacagatccccgactttatttaataagcttccgcagcggcagatgattgcggacgtcgcc

SEQ ID NO: 169 - DNA, 70 bp, synthetic construct
  source 1..70: mol_type = other DNA
acaacgcttggaaaacgtctggcacagcactaataagcttgatcgggcggtgatcctcaatgccgggatt

SEQ ID NO: 170 - DNA, 67 bp, synthetic construct
  source 1..67: mol_type = other DNA
gctgaaatgtcagaatatctgtttgataagtaataatcttagcaagcgggatgccaaagaactggtt

SEQ ID NO: 171 - DNA, 70 bp, synthetic construct
  source 1..70: mol_type = other DNA
cagcagtatctcaggctgaaagcccagcattaataagcttctgctgttttaccggatgggtgatttttat

SEQ ID NO: 172 - DNA, 70 bp, synthetic construct
  source 1..70: mol_type = other DNA
tgtaatgaatgcggggccgattatccgcgctaataagcttcagtgcagtgcctgtcatgcctggaacacc

SEQ ID NO: 173 - DNA, 70 bp, synthetic construct
  source 1..70: mol_type = other DNA
atttcgggtcttgggggtagcggcagttactaataagcttcaactggcggtgctggagcaggagtatcag

SEQ ID NO: 174 - DNA, 70 bp, synthetic construct
  source 1..70: mol_type = other DNA
attctggtgcaaagtaccggtatggcacagtaataagcttatgaccctgtcgcaaaagtttggtattgcg

SEQ ID NO: 175 - DNA, 70 bp, synthetic construct
  source 1..70: mol_type = other DNA
tgtgtcagtgaaatcggtgagctacaaaattaataagctttgcttgctggcttctcaagcggtcagcagg

SEQ ID NO: 176 - DNA, 70 bp, synthetic construct
  source 1..70: mol_type = other DNA
gatggtttccacatccatggaacggtcttcaagcttattacaggcgcatgatggagcctttaccaaattg

SEQ ID NO: 177 - DNA, 70 bp, synthetic construct
  source 1..70: mol_type = other DNA
agggatttgatagcaaagggattttccgccaagcttattacatgacgacgaggcaatcgcggccggaaag

SEQ ID NO: 178 - DNA, 70 bp, synthetic construct
  source 1..70: mol_type = other DNA
ttcaagctggttttcttccagccagcgcagaagcttattacgtatctttcagagaaaaacgggcgcacag

SEQ ID NO: 179 - DNA, 70 bp, synthetic construct
  source 1..70: mol_type = other DNA
ttccgtcacgcccgcatagatgagtttcagaagcttattataaatcatccactttggtgcggatgcatcc

SEQ ID NO: 180 - DNA, 70 bp, synthetic construct
  source 1..70: mol_type = other DNA
cgcatccacctgatgggtttgtgctgtctcaagcttattagtcaagaaaagcctgatgttcagcttcgcg

SEQ ID NO: 181 - DNA, 70 bp, synthetic construct
  source 1..70: mol_type = other DNA
gctggcacctagccagatctccagtaacacaagcttattagcctttatcgcgttttaaattctctggcgt

SEQ ID NO: 182 - DNA, 73 bp, synthetic construct
  source 1..73: mol_type = other DNA
gtgccggcagaggcttcaatcaggcgctcaccaagcttattacaagcgcaaaggatctagtgtctcggcgaca

SEQ ID NO: 183 - DNA, 70 bp, synthetic construct
  source 1..70: mol_type = other DNA
ctccgtgaagacgtccagcatcaggctggtaagcttattacgggcgactatgcaggacaaatgcgcgctg

SEQ ID NO: 184 - DNA, 70 bp, synthetic construct
  source 1..70: mol_type = other DNA
gcgcacttcgttattgagtaactcgcactcaagcttattagtgctcgctttgggtaatttgttgttttgc

SEQ ID NO: 185 - DNA, 70 bp, synthetic construct
  source 1..70: mol_type = other DNA
gacctgcggcaatattattccgacctcttgtaataagcttaaaacgcaggagggagtgggttatatttat

SEQ ID NO: 186 - DNA, 70 bp, synthetic construct
  source 1..70: mol_type = other DNA
attaccgtcgggttggttacgcgcccgattaagcttattagaatgcacggtacgcttcacctttcacttc

SEQ ID NO: 187 - DNA, 70 bp, synthetic construct
  source 1..70: mol_type = other DNA
aatcgagtatggcgagcagttttccacgctaagcttattaggcgatacgatgcaccagtacgcgcgtctt

SEQ ID NO: 188 - DNA, 70 bp, synthetic construct
  source 1..70: mol_type = other DNA
ggcgacgtccgcaatcatctgccgctgcggaagcttattaaataaagtcggggatctgttcctgaagcgc

SEQ ID NO: 189 - DNA, 70 bp, synthetic construct
  source 1..70: mol_type = other DNA
aatcccggcattgaggatcaccgcccgatcaagcttattagtgctgtgccagacgttttccaagcgttgt

SEQ ID NO: 190 - DNA, 67 bp, synthetic construct
  source 1..67: mol_type = other DNA
aaccagttctttggcatcccgcttgctaagattattacttatcaaacagatattctgacatttcagc

SEQ ID NO: 191 - DNA, 70 bp, synthetic construct
  source 1..70: mol_type = other DNA
ataaaaatcacccatccggtaaaacagcagaagcttattaatgctgggctttcagcctgagatactgctg

SEQ ID NO: 192 - DNA, 70 bp, synthetic construct
  source 1..70: mol_type = other DNA
ggtgttccaggcatgacaggcactgcactgaagcttattagcgcggataatcggccccgcattcattaca

SEQ ID NO: 193 - DNA, 70 bp, synthetic construct
  source 1..70: mol_type = other DNA
ctgatactcctgctccagcaccgccagttgaagcttattagtaactgccgctacccccaagacccgaaat

SEQ ID NO: 194 - DNA, 70 bp, synthetic construct
  source 1..70: mol_type = other DNA
cgcaataccaaacttttgcgacagggtcataagcttattactgtgccataccggtactttgcaccagaat

SEQ ID NO: 195 - DNA, 70 bp, synthetic construct
  source 1..70: mol_type = other DNA
cctgctgaccgcttgagaagccagcaagcaaagcttattaattttgtagctcaccgatttcactgacaca

SEQ ID NO: 196 - DNA, 39 bp, synthetic construct
  source 1..39: mol_type = other DNA
gtaaaacgacggccagtctactgcgtatgcattgcagac

SEQ ID NO: 197 - DNA, 42 bp, synthetic construct
  source 1..42: mol_type = other DNA
gtaaaacgacggccagtccctctcagagactaaaagcattgc

SEQ ID NO: 198 - DNA, 50 bp, synthetic construct
  source 1..50: mol_type = other DNA
gtaaaacgacggccagtaccagtttatactgtacacaataacagtaatgg

SEQ ID NO: 199 - DNA, 39 bp, synthetic construct
  source 1..39: mol_type = other DNA
gtaaaacgacggccagtctgatgcgtctgatcgatatgc

SEQ ID NO: 200 - DNA, 41 bp, synthetic construct
  source 1..41: mol_type = other DNA
gtaaaacgacggccagtataggttgcgatcattaatgcgac

SEQ ID NO: 201 - DNA, 41 bp, synthetic construct
  source 1..41: mol_type = other DNA
gtaaaacgacggccagttcgattaccggcaacctaaaaagc

SEQ ID NO: 202 - DNA, 38 bp, synthetic construct
  source 1..38: mol_type = other DNA
gtaaaacgacggccagtgctgacgccgcaaaaacttgc

SEQ ID NO: 203 - DNA, 38 bp, synthetic construct
  source 1..38: mol_type = other DNA
gtaaaacgacggccagtttgaagcgcgtaaagacatgc

SEQ ID NO: 204 - DNA, 39 bp, synthetic construct
  source 1..39: mol_type = other DNA
gtaaaacgacggccagtgcaggaaatgcctttccaactg

SEQ ID NO: 205 - DNA, 46 bp, synthetic construct
  source 1..46: mol_type = other DNA
gtaaaacgacggccagtaagccagcagagtgaataaggataaggac

SEQ ID NO: 206 - DNA, 39 bp, synthetic construct
  source 1..39: mol_type = other DNA
gtaaaacgacggccagtaagattgatttcacccgcgagc

SEQ ID NO: 207 - DNA, 38 bp, synthetic construct
  source 1..38: mol_type = other DNA
gtaaaacgacggccagtatttcccggttggcatctctg

SEQ ID NO: 208 - DNA, 43 bp, synthetic construct
  source 1..43: mol_type = other DNA
gtaaaacgacggccagttcgctggtgctgtagaaatttatgat

SEQ ID NO: 209 - DNA, 37 bp, synthetic construct
  source 1..37: mol_type = other DNA
gtaaaacgacggccagttggtggcaaatccgccaagc

SEQ ID NO: 210 - DNA, 42 bp, synthetic construct
  source 1..42: mol_type = other DNA
gtaaaacgacggccagtgcgtaaacttatttgacgtgtaccg

SEQ ID NO: 211 - DNA, 42 bp, synthetic construct
  source 1..42: mol_type = other DNA
gtaaaacgacggccagtcttgcttcataagcatcacgcaaaa

SEQ ID NO: 212 - DNA, 37 bp, synthetic construct
  source 1..37: mol_type = other DNA
gtaaaacgacggccagtgattgcctaccatgccaagc

SEQ ID NO: 213 - DNA, 39 bp, synthetic construct
  source 1..39: mol_type = other DNA
gtaaaacgacggccagtgccgcgattaacgaaatctacg

SEQ ID NO: 214 - DNA, 40 bp, synthetic construct
  source 1..40: mol_type = other DNA
gtaaaacgacggccagtagcatcagcgttacgctagtttc

SEQ ID NO: 215 - DNA, 37 bp, synthetic construct
  source 1..37: mol_type = other DNA
gtaaaacgacggccagtggaagctgtggagcacaaac

SEQ ID NO: 216 - DNA, 23 bp, synthetic construct
  source 1..23: mol_type = other DNA
cccagtttacgtgcgtagattgg

SEQ ID NO: 217 - DNA, 25 bp, synthetic construct
  source 1..25: mol_type = other DNA
gatataaagcagacgaatttgcccg

SEQ ID NO: 218 - DNA, 25 bp, synthetic construct
  source 1..25: mol_type = other DNA
agctttgatgccacaactgataacg

SEQ ID NO: 219 - DNA, 20 bp, synthetic construct
  source 1..20: mol_type = other DNA
cggaccatatgctgcacctg

SEQ ID NO: 220 - DNA, 20 bp, synthetic construct
  source 1..20: mol_type = other DNA
cgcgacgaggcaagatttgc

SEQ ID NO: 221 - DNA, 20 bp, synthetic construct
  source 1..20: mol_type = other DNA
cttcccagtcttcgcgtagc

SEQ ID NO: 222 - DNA, 20 bp, synthetic construct
  source 1..20: mol_type = other DNA
tcggtggtttcacgcagaca

SEQ ID NO: 223 - DNA, 22 bp, synthetic construct
  source 1..22: mol_type = other DNA
aaagttcagagaagcgcgtctc

SEQ ID NO: 224 - DNA, 21 bp, synthetic construct
  source 1..21: mol_type = other DNA
gcgacaacaggctgttcagac

SEQ ID NO: 225 - DNA, 27 bp, synthetic construct
  source 1..27: mol_type = other DNA
cgtcatcttaaatttgaggggtgacgg

SEQ ID NO: 226 - DNA, 25 bp, synthetic construct
  source 1..25: mol_type = other DNA
agatttcagtgccggttaactcctc

SEQ ID NO: 227 - DNA, 23 bp, synthetic construct
  source 1..23: mol_type = other DNA
ggttcatggctttgatcagacgc

SEQ ID NO: 228 - DNA, 27 bp, synthetic construct
  source 1..27: mol_type = other DNA
gcagtgaatttaagatcgggaatgatc

SEQ ID NO: 229 - DNA, 21 bp, synthetic construct
  source 1..21: mol_type = other DNA
cagttgctggcgtaaaacacc

SEQ ID NO: 230 - DNA, 23 bp, synthetic construct
  source 1..23: mol_type = other DNA
cagattactcgtctttgggcgaa

SEQ ID NO: 231 - DNA, 21 bp, synthetic construct
  source 1..21: mol_type = other DNA
ggatcgccaatttgttcgcag

SEQ ID NO: 232 - DNA, 21 bp, synthetic construct
  source 1..21: mol_type = other DNA
gccaatcagaatggcacttcc

SEQ ID NO: 233 - DNA, 22 bp, synthetic construct
  source 1..22: mol_type = other DNA
cagttaccgacgcgggataatc

SEQ ID NO: 234 - DNA, 22 bp, synthetic construct
  source 1..22: mol_type = other DNA
gtcagtcagataatgccgcaac

SEQ ID NO: 235 - DNA, 26 bp, synthetic construct
  source 1..26: mol_type = other DNA
ccgcaactttttgccagttaatttca

SEQ ID NO: 236 - DNA, 22 bp, synthetic construct
  source 1..22: mol_type = other DNA
gctgcggcgccctacgggcttg

SEQ ID NO: 237 - DNA, 61 bp, synthetic construct
  source 1..61: mol_type = other DNA
accgccggcgtaacagatgaggatcctatttcttaataactaaaaatatggtataatactc

SEQ ID NO: 238 - DNA, 21 bp, synthetic construct
  source 1..21: mol_type = other DNA
ggaggacaatccgcggaaccc

SEQ ID NO: 239 - DNA, 52 bp, synthetic construct
  source 1..52: mol_type = other DNA
ggctgtagccgcctcgccgcgttgacaattaatcatcggcatagtatatcgg

SEQ ID NO: 240 - DNA, 25 bp, synthetic construct
  source 1..25: mol_type = other DNA
gcggcgaggcggctacagccgatag

SEQ ID NO: 241 - DNA, 41 bp, synthetic construct
  source 1..41: mol_type = other DNA
agcccgtagggcgccgcagcgatactgagcacatcagcagg

SEQ ID NO: 242 - DNA, 22 bp, synthetic construct
  source 1..22: mol_type = other DNA
tcatctgttacgccggcggtag

SEQ ID NO: 243 - DNA, 43 bp, synthetic construct
  source 1..43: mol_type = other DNA
ggttccgcggattgtcctccttaagccttaggacgcttcacgc

SEQ ID NO: 244 - DNA, 75 bp, synthetic construct
  source 1..75: mol_type = other DNA
taataaatttgcggccgctaatacgactcactatagggagaggatccgcggatactgagcacatcagcaggacgc

SEQ ID NO: 245 - DNA, 23 bp, synthetic construct
  source 1..23: mol_type = other DNA
cagtactgcgatgagtggcaggg

SEQ ID NO: 246 - DNA, 27 bp, synthetic construct
  source 1..27: mol_type = other DNA
gtggtgaccaggaatgttcagagcatc

SEQ ID NO: 247 - DNA, 23 bp, synthetic construct
  source 1..23: mol_type = other DNA
gcataaaagacggctgctggagg

SEQ ID NO: 248 - DNA, 83 bp, synthetic construct
  source 1..83: mol_type = other DNA
ccacccaacccccacaatattttaattcagaattcatcagtataagaattcgttgacaattaatcatcggcatagtatatcgg

SEQ ID NO: 249 - DNA, 68 bp, synthetic construct
  source 1..68: mol_type = other DNA
gtttgagaccagtctgggcaacaaagcaagattccatctctaaagaattccggcctggtgatgatggc

SEQ ID NO: 250 - DNA, 83 bp, synthetic construct
  source 1..83: mol_type = other DNA
gaggtccagggaaaggatgtgattggctcatggggcatatattgtgaattcgttgacaattaatcatcggcatagtatatcgg

SEQ ID NO: 251 - DNA, 31 bp, synthetic construct
  source 1..31: mol_type = other DNA
gcagtaatacaggggcttttcaagactgaag

(19) United States
(12) Patent Application Publication
(10) Pub. No.: US 2025/0075231 A1
(43) Pub. Date: Mar. 6, 2025

(54) METHODS OF EDITING NUCLEIC ACID SEQUENCES

(71) Applicant: United Kingdom Research and Innovation, Swindon, Wiltshire (GB)

(72) Inventors: Jerome F. ZURCHER, Swindon (GB); Louise F. H. FUNKE, Swindon (GB); Askar A. KLEEFELDT, Swindon (GB); Jakob BIRNBAUM, Swindon (GB); Julius FREDENS, Swindon (GB); Martin SPINCK, Swindon (GB); Jason W. CHIN, Swindon (GB)

(21) Appl. No.: 18/695,095

(22) PCT Filed: Nov. 3, 2022
(86) PCT No.: PCT/EP2022/080675

§ 371 Date: Mar. 25, 2024

(30) Foreign Application Priority Data
Nov. 3, 2021 (GB) 2115820.9

(51) Int. Cl. C12N 15/90; C12N 9/22; C12N 15/11; C12N 15/70

(52) U.S. Cl. CPC C12N 15/902; C12N 9/22; C12N 15/111; C12N 15/70; C12N 2310/20

(57) ABSTRACT

In an aspect, the present invention relates to methods of introducing a sequence of interest into a target nucleic acid. The invention also relates to methods of assembling nucleic acid sequences comprising iterating the methods of introducing a sequence of interest into a target nucleic acid, as well as assembly of replicons encoding larger amounts heterologous nucleic acid.

### FIELD OF THE INVENTION

In an aspect, the present invention relates to methods of introducing a sequence of interest into a target nucleic acid. The invention also relates to methods of assembling nucleic acid sequences comprising iterating the methods of introducing a sequence of interest into a target nucleic acid.

### BACKGROUND OF THE INVENTION

Strategies for replacing genomic DNA with synthetic DNA¹⁻¹² enable genome engineering and provide a basis for powerful technologies to create entirely synthetic genomes that cannot be created by editing methodologies alone. Genome synthesis has been used to create synthetic genomes for two organisms: mycoplasma (1 Mb), where it has been used to investigate genome minimization¹³, and E. coli (4 Mb), where it has been used to create a recoded organism¹⁴. The work in E. coli removed over 18,000 synonymous codons, to create an organism with a compressed genetic code which uses just 61 codons to encode the canonical amino acids. The recoded E. coli with a compressed genetic code provides a foundation for the creation of virus resistant cells, and for sense codon reassignment for non-canonical amino acid incorporation and encoded non-canonical polymer synthesis¹⁵. Efforts to synthesize other genomes and strategies to recode, minimize or rearrange genomes are under way^{9,10,16-18}.

The E. coli genome synthesis was based on REXER (Replicon Excision Enhanced Recombination)⁵. In REXER (FIG. 1a) a bacterial artificial chromosome (BAC)—containing an insert composed of the synthetic DNA of interest flanked by a double selection cassette (composed of a positive and negative selection marker) and 50-152 bp regions of homology to the genome—is transformed into cells containing a helper plasmid that encodes CRISPR/Cas9 and the lambda red recombination machinery. A single clone containing the correct BAC is isolated and made competent and then a spacer array is introduced into the competent cells to activate Cas9 mediated excision of the insert containing the synthetic DNA from the BAC. The spacer sequences are designed such that the spacer RNAs base pair with the unique sequences in the homology region of the insert and precisely cleave at the junctions between the insert and the BAC backbone (FIG. 1b). The precisely excised insert is then used, by the lambda red recombination machinery, to insert the synthetic DNA into the genome in place of the corresponding genomic DNA. Distinct double selection cassettes in the genome and synthetic DNA are used to select for the desired integration (FIG. 1a). It takes four days to go from cells with an appropriately marked genome to having clonal colonies on a post-REXER agar plate.

A single step of REXER has been used to replace up to 136 kb of the genome with synthetic, recoded DNA. The genome produced from one step of REXER provides a template for the next round of REXER, and iteration of REXER (Genome interchange stepwise synthesis, GENESIS) enables larger sections of the E. coli genome to be replaced with synthetic DNA. 38 REXER steps—each requiring the design, synthesis, cloning and validation of bespoke spacer pairs—were used to replace the entire E. coli genome (across seven strains) with synthetic recoded DNA. The recoded DNA was then compiled, by conjugation, into a single strain to create a recoded organism.

Strategies to further simplify and accelerate the introduction of synthetic DNA into the genome of E. coli will be key to future large-scale genome engineering, and genome synthesis efforts.

### SUMMARY OF THE INVENTION

In an aspect of the invention, there is provided a method of introducing a sequence of interest into a target nucleic acid, the method comprising
a) providing a host cell
   said host cell comprising an episomal replicon,
said episomal replicon comprising a backbone sequence and a donor nucleic acid sequence,
wherein said donor nucleic acid sequence comprises in order: 5′—homologous recombination sequence 1—sequence of interest—homologous recombination sequence 2—3′,
wherein the backbone sequence comprises a first excision site positioned adjacent to homologous recombination sequence 1 and a second excision site positioned adjacent to homologous recombination sequence 2,
   said host cell further comprising a target nucleic acid;
b) providing helper protein(s) capable of supporting nucleic acid recombination in said host cell;
c) providing an RNA-guided DNA endonuclease;
d) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
e) inducing excision of said donor nucleic acid sequence by the RNA-guided DNA endonuclease; and
f) incubating to allow recombination between the excised donor nucleic acid and said target nucleic acid.

The RNA-guided DNA endonuclease may be a CRISPR-Cas nuclease, the first RNA molecule may comprise a spacer specific for the first excision site, and the second RNA molecule may comprise a spacer specific for the second excision site. The CRISPR-Cas nuclease may be Cas9. The first RNA molecule and/or the second RNA molecule may be encoded by the episomal replicon.

In an embodiment, each terminus of the excised nucleic acid comprises nucleic acid sequence derived from the backbone sequence. The excised donor nucleic acid may comprise 6 or fewer base pairs of nucleic acid sequence derived from the backbone sequence at each terminus.

In an embodiment, the episomal replicon is a bacterial artificial chromosome. The episomal replicon may be delivered to the host cell by conjugative transfer.

The target nucleic acid may be the genome of the host cell. The host cell may be a prokaryotic cell, such as Escherichia coli.

In an aspect of the invention, there is provided a method of assembling a nucleic acid sequence, the method comprising:
(i) performing the steps of a method of introducing a sequence of interest into a target nucleic acid of the invention, to introduce a first donor nucleic acid sequence into a first target nucleic acid in order to create a second target nucleic acid; and
(ii) performing the steps of a method of introducing a sequence of interest into a target nucleic acid of the invention, to introduce a second donor nucleic acid sequence into the second target nucleic acid in order to create a third target nucleic acid. Part (i) and part (ii) may be iterated.

In an embodiment, the sequence of the first RNA molecule for part (i) is the same for each iteration and/or the sequence of the second RNA molecule for part (i) is the same for each iteration; and the sequence of the first RNA molecule for part (ii) is the same for each iteration and/or the sequence of the second RNA molecule for part (ii) is the same for each iteration.

In an embodiment, the method further comprises:
(iii) performing the steps of a method of introducing a sequence of interest into a target nucleic acid of the invention, to introduce a third donor nucleic acid sequence into the third target nucleic acid in order to create a fourth target nucleic acid;
iterating parts (i), (ii), and (iii), and wherein
   the sequence of the first RNA molecule for part (iii) is the same for each iteration and/or
   the sequence of the second RNA molecule for part (iii) is the same for each iteration.

In a particular embodiment, part (i) comprises the use of a donor-nucleic-acid-sequence-encoding episomal replicon comprising a first backbone sequence, and part (ii) comprises the use of a donor-nucleic-acid-sequence-encoding episomal replicon comprising a second backbone sequence, wherein
the first backbone sequence comprises a first marker or set of markers, encodes the first RNA molecule specific for the first excision site within said first backbone sequence, and encodes the second RNA molecule specific for the second excision site within said first backbone sequence; and
the second backbone sequence comprises a second marker or set of markers, encodes the first RNA molecule specific for the first excision site within said second backbone sequence, and encodes the second RNA molecule specific for the second excision sites within said second backbone sequence; wherein
   the first marker or set of markers is different from the second marker or set of markers.

In a further aspect of the invention, there is provided a method for constructing an episomal replicon, comprising the steps of:
a) providing a donor episomal replicon, said replicon comprising:
a backbone, said backbone comprising universal spacer sequences,
a first homology region HRn which is specific for an integration step n, and a second, universal, homology region uHR,
a first excision site positioned adjacent to HRn and a second excision site positioned adjacent to uHR;
a donor nucleic acid DNAn positioned between HRn and uHR; and
a double selection cassette, comprising positive and negative selection markers;
b) providing a host cell comprising an assembly episomal replicon comprising a double selection cassette comprising positive and negative selection markers, flanked by HRn and uHR, the double selection cassette in the assembly replicon comprising different markers to the selection cassette in the donor replicon;
c) providing helper protein(s) capable of supporting nucleic acid recombination in said host cell;
d) providing an RNA-guided DNA endonuclease;
e) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
f) inducing excision of said donor nucleic acid sequence DNAn by the RNA-guided DNA endonuclease in the host cell; and
g) incubating to allow recombination between the excised donor nucleic acid and said assembly replicon to form a second assembly replicon, which comprises the nucleic acid DNAn.

The RNA-guided DNA endonuclease may be a CRISPR-Cas nuclease, the first RNA molecule may comprise a spacer specific for the first excision site, and the second RNA molecule may comprise a spacer specific for the second excision site. The CRISPR-Cas nuclease may be Cas9. The first RNA molecule and/or the second RNA molecule may be encoded by the episomal replicon.

In an embodiment, each terminus of the excised nucleic acid comprises nucleic acid sequence derived from the backbone sequence. The excised donor nucleic acid may comprise 12, 10, 8, 6, 4 or 2 base pairs of nucleic acid sequence derived from the backbone sequence at each terminus. Preferably, the excised donor nucleic acid comprises 6 or fewer base pairs of nucleic acid sequence derived from the backbone sequence at each terminus.

In an embodiment, the episomal replicon is a bacterial artificial chromosome. The episomal replicon may be delivered to the host cell by conjugative transfer.

In a preferred aspect of the invention, the donor episomal replicon is comprised in a donor host cell and the cell assembly replicon is comprised in a recipient host cell. Conjugation between the donor and recipient host cells can advantageously transfer the donor episomal replicon to the recipient host cell. The donor host cell preferably comprises a non-transferrable F′ plasmid, such that the F′— plasmid is not transferred to the recipient host cell. Preferably, the F′ plasmid is non-transferable through oriT deletion. The donor episome is transferrable, and may contain an oriT.

Selection for the host cell can be accomplished as described, but employing positive and negative selection markers present in recombined donor and assembly replicon DNA.

The target nucleic acid may be the genome of the host cell. The host cell may be a prokaryotic cell, such as Escherichia coli.

In one embodiment, the donor nucleic acid comprises a homology region HRn+1, and the method further comprises a further step (h) of introducing into the host cell a further donor episomal replicon comprising a donor nucleic acid DNAn+1, and inducing excision of said donor nucleic acid sequence DNAn+1 by the RNA-guided DNA endonuclease in the host cell; and incubating to allow recombination between the excised donor nucleic acid DNAn+1 and said second assembly replicon to form a third assembly replicon, which comprises the nucleic acid DNAn and nucleic acid DNAn+1.

Said method steps can be iteratively repeated.

The replicon provided in this aspect of the invention may be used in the foregoing aspects which describe methods of assembling a nucleic acid sequence.

In an advantageous embodiment of all aspects of the present invention, the host cell is lacking competent recA and/or recO. Preferably, the host cell lacks recA (ΔrecA).

### BRIEF DESCRIPTION OF THE DRAWINGS

FIG. 1|The steps in REXER-mediated integration of ˜100 kb of synthetic DNA into the E. coli genome using homology region (HR)-specific spacers, and the relationship between the cuts directed by HR-specific spacers and universal spacers.

a, REXER allows integration of more than 100 kb of synthetic DNA (pink) into the genome, either through replacement of the genomic DNA (as shown here) or by insertion into the genome. A bacterial artificial chromosome (BAC) containing the synthetic DNA of interest is electroporated into competent cells with a suitably marked genome, the cells also contain a helper plasmid encoding the Cas9 protein and the lambda red recombination components. The cell is then induced with arabinose to express the helper plasmid genes and made electrocompetent again. HR-specific spacer arrays (either plasmid based as shown, or as linear DNA) are then electroporated into the cell, leading to CRISPR/Cas9 mediated in vivo excision of the synthetic DNA flanked by a double selection cassette and HRs to the genome, from the BAC. The lambda red recombination machinery then uses the HRs to direct the integration of the excised DNA into the genome. Triangles denote the Cas9 cleavage sites at the HRs (grey boxes) flanking the synthetic DNA. +1, blue is kan^{R}; −1, yellow is rpsL; +2, green is cat; −2, pink is sacB; +3, dark blue is tet^{R}; −3, purple is pheS*; +4, orange is amp^{R}; b, Previously, we designed spacer RNAs specific for each HR flanking the genomic locus for recombination. The BAC sequence flanking the synthetic DNA insert contains a constant PAM sequence (black box). Directing Cas9 with HR-specific spacer RNA allows precise excision of the synthetic DNA at the ends of HR1 and HR2. c, Universal spacer RNAs direct Cas9 to the constant sequence of the BAC backbone. To create a universal cut site for any BAC independent of the REXER locus, Cas9 is directed to the PAM sequences directly flanking the HRs. This adds an additional 6 bp non-homologous sequences to both ends of the excised DNA fragment.

FIG. 2|REXER with universal spacers results in scarless genomic integration of large synthetic DNA fragments.

a, BAC backbones used for total synthesis of the E. coli genome. The two BACs, referred to as odd numbered and even numbered BACs, contain distinct positive and negative selection cassettes, which allows REXER to be iterated. We designed Universal1 spacers for all odd numbered BACs and Universal2 spacers for all even numbered BACs (Table 3 and SEQ ID NOs: 24 and 25). One universal spacer RNA (blue) targets the sequence in the BAC backbones 5′ to the insert; this sequence is common to both backbones. Universal spacer RNAs targeting the BAC backbone 3′ to the insert; These 3′ sequences are distinct in the two backbones, and therefore two different spacer sequences (yellow and red, respectively) were designed. Cut sites are indicated with coloured triangles, PAM sequences are shown in black boxes, selection cassettes are shown in coloured arrows, and synthetic DNA is shown in pink. b, Verification by genotyping of the 5′ and 3′ genomic integration sites after REXER using universal spacers at five genomic loci. At the 5′ locus, a double selection cassette is removed from the genome upon successful replacement by REXER, while another double selection cassette is inserted at the 3′ locus. 11 post-REXER clones were genotyped for each experiment. Triangles indicate the size of the expected PCR product at each locus before (white) and after (black) REXER. c, Sequence verification of the ends of the integration sites after REXER using universal spacer RNA. The excised synthetic DNA flanked by HRs and 6 bp non-homology sequences (tilted) is shown above the sequence that is expected for scarless integration. Five post-REXER clones were sequenced for each experiment. We did not observe integration of the non-homologous termini for any clone and neither did any point mutations appear. Coloured triangles indicate the Cas9 cut sites by the respective spacer sequences described in panel a. d, Compiled recoding landscapes of REXER with HR-specific and universal spacers, respectively. We performed REXER, replacing 95.6 kb of wildtype genomic DNA with synthetic DNA (100k24). The synthetic DNA is mostly homologous to the corresponding genomic DNA, except for 410 codons that have been replaced with synonymous codons¹⁴. Cas9 cleavage was initiated with HR-specific or universal spacer RNAs. 10 post-REXER clones were fully sequenced by NGS for each experiment (20 in total from two independent repeat experiments). The compiled recoding landscape graphs show the average frequency at which each recoded codon was integrated across the genomic locus. Overall, REXER with universal spacer RNA yielded 8 clones with complete replacement of all 410 codons, whereas REXER with HR-specific spacer RNA yielded 4 completely recoded clones at this region (Recoding landscapes of individual clones in FIG. 5)

FIG. 3|Conjugation coupled with programmed excision for enhanced recombination (CONEXER).

Coupling episome transfer, excision of synthetic DNA with universal spacers, and homology directed recombination creates a rapid, simplified and standardised method for large scale genome engineering and genome synthesis. a, The BAC with a universal spacer array (grey bars) and oriT sequence (red arrow) is transferred from donor cells to recipient cells with the aid of a non-transferable F′ plasmid, via conjugative transfer for 1 h. Recipient cells contain the helper plasmid and appropriately marked genome (as in REXER). Recipient cells that have acquired the BAC are then selected, and donor cells removed, by selection for both +3 and +1 (1.5 h with arabinose to induce Cas9 and the lambda red components, followed by 2.5 h with glucose to stop further expression). Replacement of genomic DNA with synthetic DNA is then selected for by additionally selecting for loss of −2; selection for loss of −3 ensures loss of the BAC backbone. The selectable markers are +1, blue, kan^{R}; −1, yellow, rpsL; +2, green, cat; −2, pink, sacB; +3, dark blue, tet^{R}; −3, purple, pheS*. b, The compiled recoding landscape (analogous to FIG. 2d) of 84 clones from CONEXER with 100k24.

FIG. 4|Genome map

FIG. 5|Compiled recoding landscapes of REXER with HR-specific and universal spacers, respectively. The recoding landscapes of individual clones are shown.

FIG. 6|BAC Stepwise Insertion Synthesis (BASIS)

BAC stepwise insertion synthesis (BASIS) for iterative assembly of large DNA in BACs.

a, The donor BAC contains HRn and uHR, homologous to the recipient BAC. The BAC backbone contains oriT and universal spacers. uHR is a universal homology region for all steps of insertion. HRn is specific for the nth step of insertion. The BAC insert contains HRn+1, which serves as HRn for the (n+1)th step. The BAC contains a double selection cassette, −3, +3 shown. The assembly BAC contains a distinct double selection cassette, −1, +1 shown, flanked by HRn and uHR. This DNA insert is excised from the donor BAC and inserted into the assembly BAC in the recipient cell. Green triangles indicate cut sites for Cas9 excision. Note in the main text HRn is described as HR1. In the example shown, the selectable markers are +1, blue, kan^{R}; −1, yellow, rpsL; +3, purple, hygro_{R}; −2, orange, PheS; +4, petrol, Gentamycin_{R}.

b, BASIS workflow. The donor BAC is delivered by conjugation to the recipient cell containing the assembly BAC and expressing Cas9 and lambda red components. The insert is excised from the donor BAC and inserted into the recipient BAC, as shown in (a). Iteration of this process, using alternating sets of markers, allows for the insertion of n DNA fragments into the assembly BAC.

c, Three BACs encoding for segments of the CFTR gene were assembled in yeasto. The full CFTR gene sequence was reconstituted through iterative BASIS and verified by next generation sequencing (NGS).

d, Human BACs covering the indicated region of chromosome 21 were employed as substrate for successive assembly. Intermediate and final assembly products were verified by NGS. The final 503 Kbp BAC contained a 495 Kbp human DNA insert; this BAC can serve as the substrate for further iterative insertion, thereby enabling even larger stretches of human DNA to be assembled in episomes.

FIG. 7|Deletion of recA from the host genome increases the frequency of fully recoded clones in CONEXER mediated genomic replacement

a, Screening of KO strains in CONEXER mediated replacement of genomic fragment 100k24 for increased frequency of fully recoded clones reveals deletion of recA and recO improve intact integration of synthetic DNA.

b, Upon deletion of recA the frequency of fully recoded clones following CONEXER mediated genome replacements is increased in all tested fragments (100k24-100k28). Colony forming units (CFU) recorded for these experiments are shown in FIG. 9.

FIG. 8|Continuous genome synthesis

a, Conjugation-delivered and episome-excised, synthetic DNA recombines replacing a large fragment (100k24) of an appropriately marked genome (−2/+2 at LS23). Selection ensures only cells that lost −2/+2 (at LS23) and integrated −1/+1 (at LS24) survive. Clones from the selection are pooled and undergo a subsequent round of CONEXER mediated genome replacement (100k25). Selection ensures only cells that lost −1/+1 (at LS24) and integrated −2/+2 (at LS25) survive. This process was repeated three more times (100k26, 100k27 & 100k28), five times in total, until a population of cells with −1/+1 (at LS28) is obtained. A subset of those cells are expected to have continuously integrated synthetic DNA over the entire 500 Kbp region. The selectable markers are +1, blue, kan_{R}; −1, yellow, rpsL; +2, green, cat; −2, pink, sacB.

b, The compiled recoding landscape of 182 clones from continuous genome synthesis of 100k24-100k28. 19 out of the 182 sequenced clones were fully recoded over the whole 500 Kbp section of the genome.

FIG. 9|Colony forming units for CONEXER mediated genomic replacement in different genomic backgrounds

Screening of KO strains in CONEXER mediated replacement of genomic section 100k24. Colony forming units (CFU) obtained in each experiment are indicated. Deletion of recA leads to a reduction of CFU.

FIG. 10|Continuous synthesis of 500 Kbp of the E. coli genome

Conjugation enhanced replacement of 100 Kbp genome sections with synthetic DNA enables continuous genome synthesis (FIG. 5). Episome-excised, synthetic DNA recombines replacing a large section (100k24) of an appropriately marked genome (−2/+2 at LS23). Selection ensures only cells that lost −2/+2 (at LS23) and integrated −1/+1 (at LS24) survive. Clones from the selection are pooled and undergo a subsequent round of CONEXER mediated genome replacement (100k25). Selection ensures only cells that lost −1/+1 (at LS24) and integrated −2/+2 (at LS25) survive. This process is iterated 3 more times (100k26, 100k27 & 100k28) until a population of cells with −1/+1 (at LS28) is obtained. A subset of those cells is expected to have continuously integrated synthetic DNA over a region of 500 Kbp. The selectable markers are +1, blue, kan^{R}; −1, yellow, rpsL; +2, green, cat; −2, pink, sacB. Each step of >100 Kbp synthetic DNA integration by CONEXER takes two days. Continuous synthesis of 500 Kbp can therefore be achieved in 10 days.

### DETAILED DESCRIPTION

The methods disclosed in the prior art, such as REXER, require a new set of homology region (HR)-specific spacers to be cloned for each locus that is targeted; these spacers can be challenging to clone, and spacer cloning can be expensive and time consuming. For instance, the recent E. coli genome synthesis required the cloning of 78 unique spacers. Each new set of spacers must be designed to avoid undesired cutting of the target nucleic acid. Additionally, varying the spacer sequence can affect the excision efficiency and this may contribute to variation in the efficiency of REXER at distinct genomic loci. The requirement for HR-specific spacer RNA complicates the workflow and may limit the scalability of REXER.

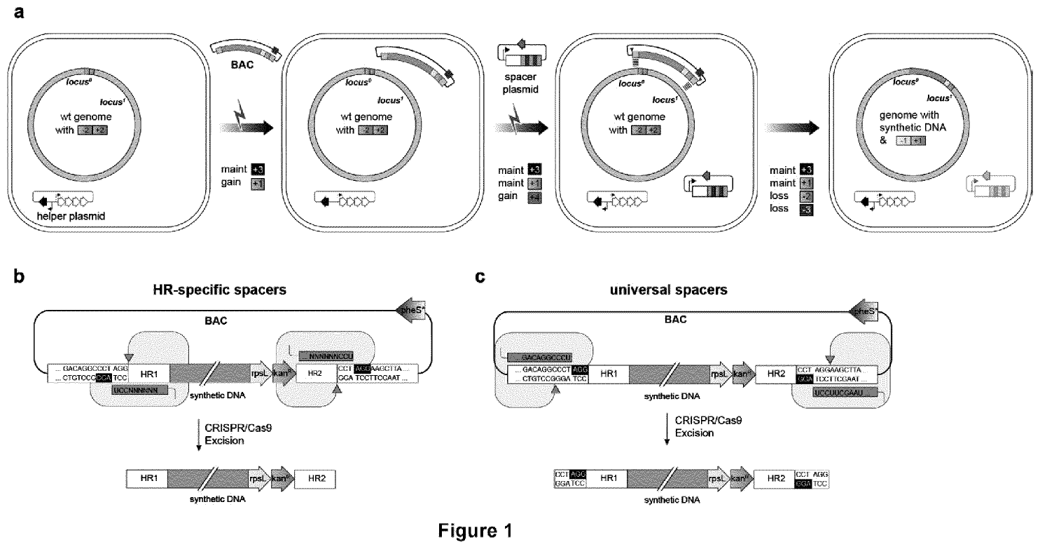

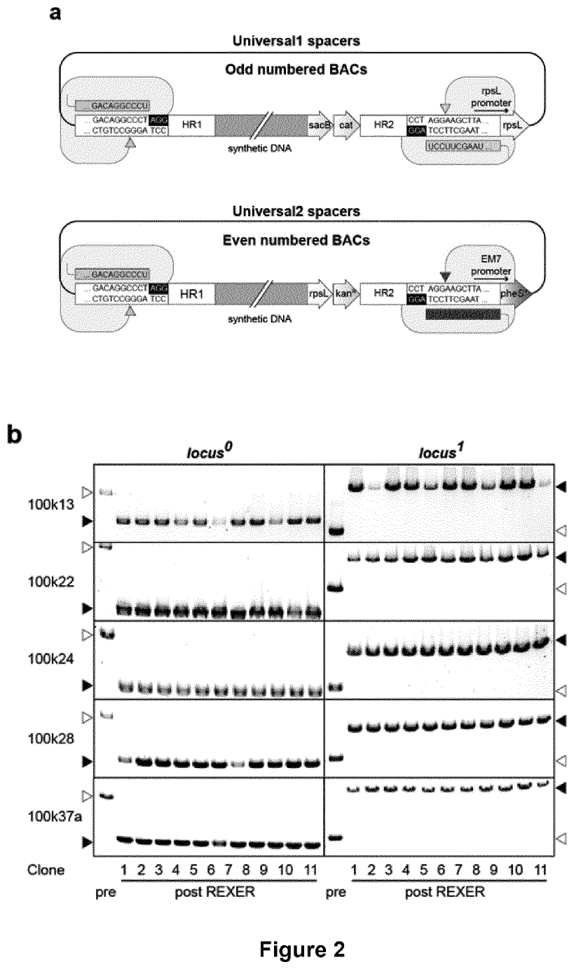

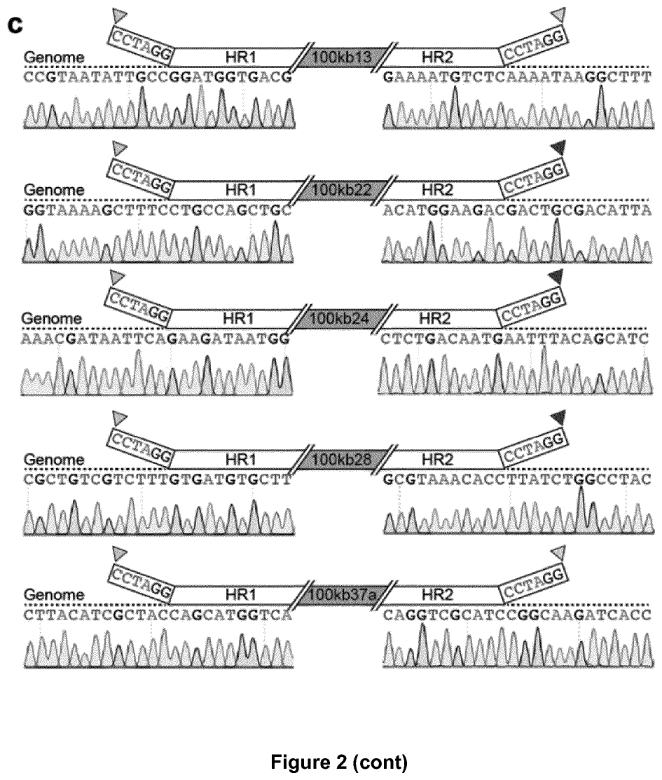

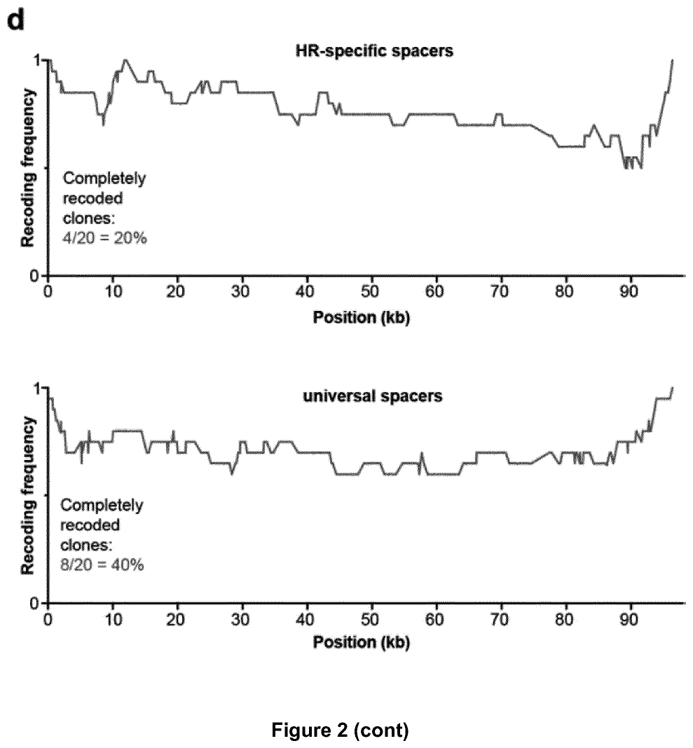

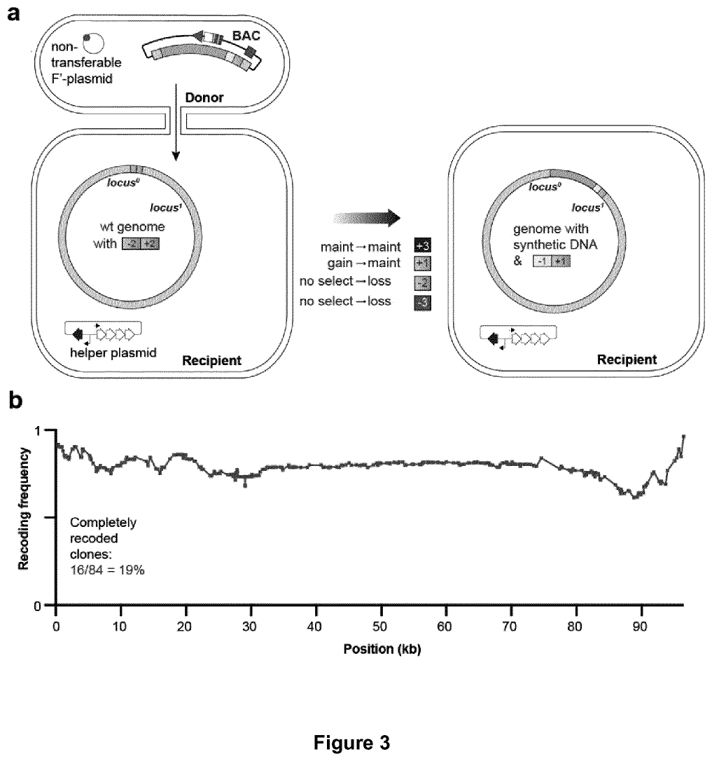

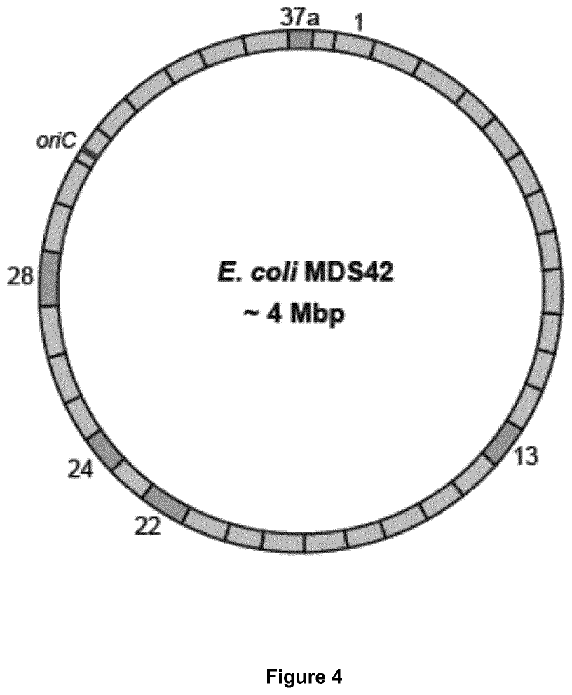

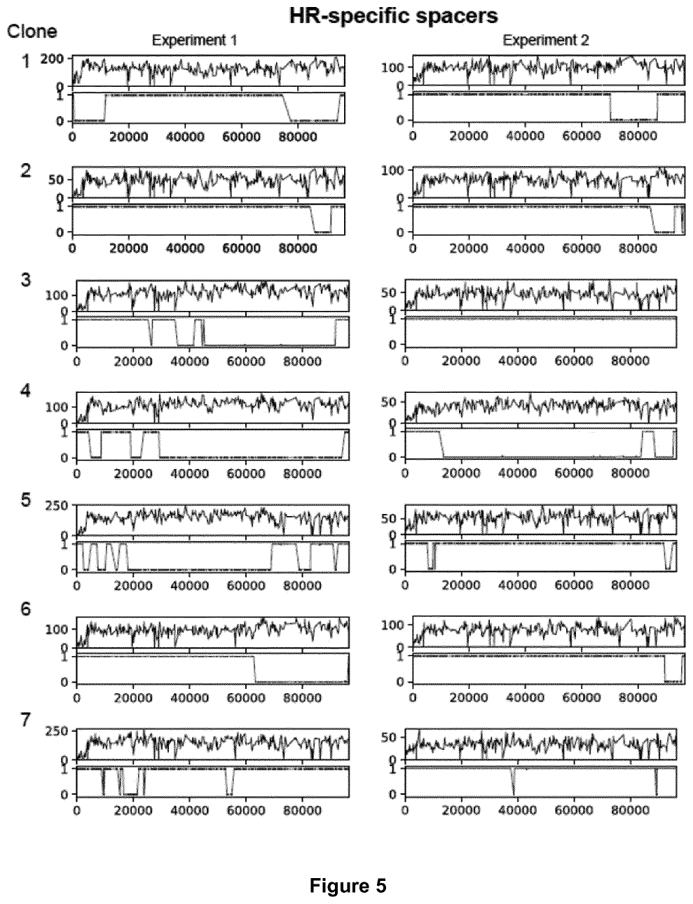

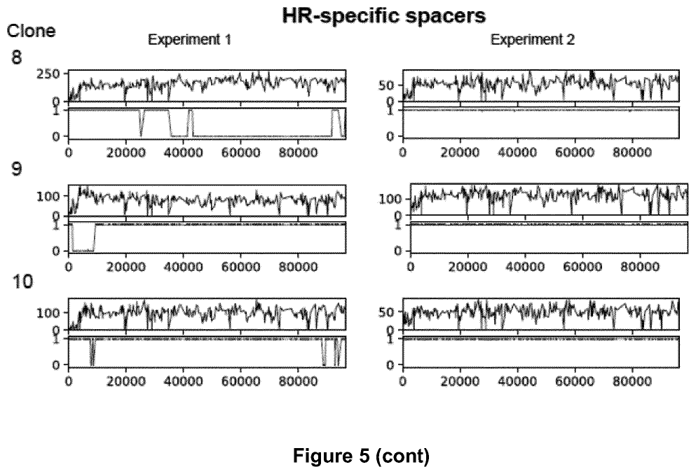

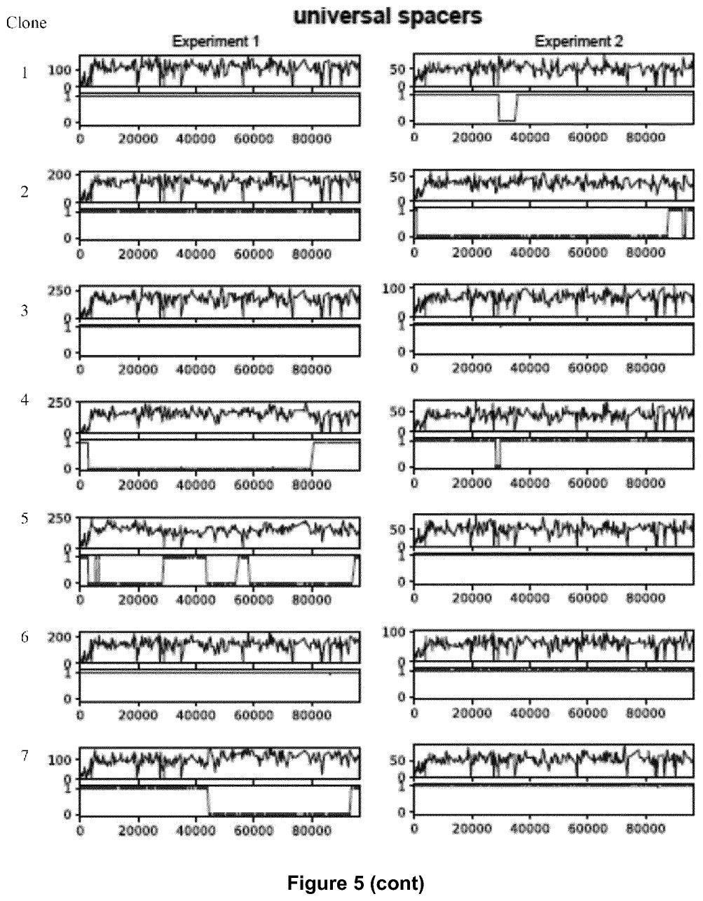

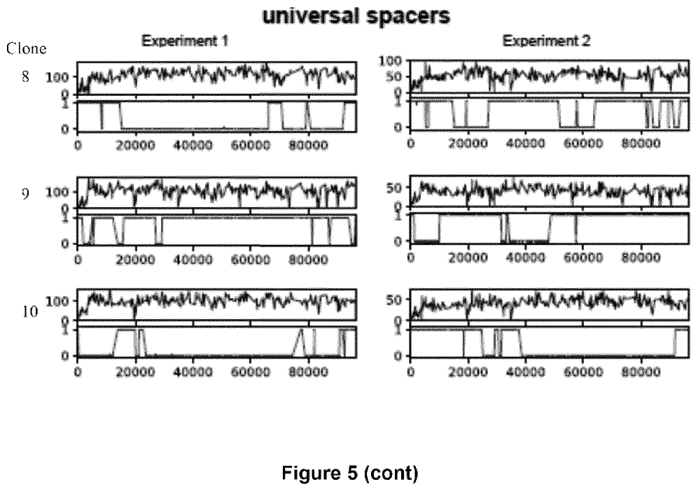

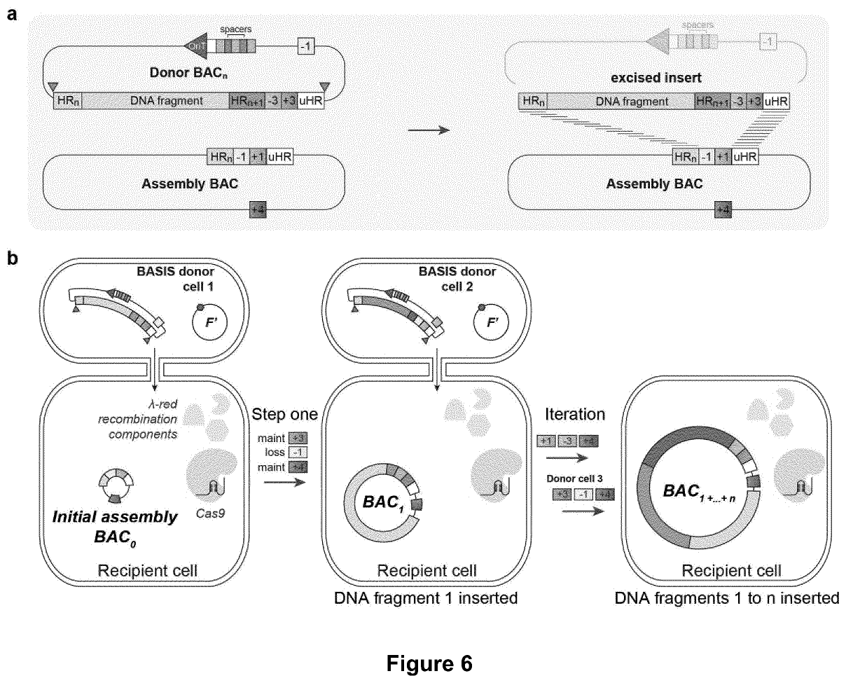

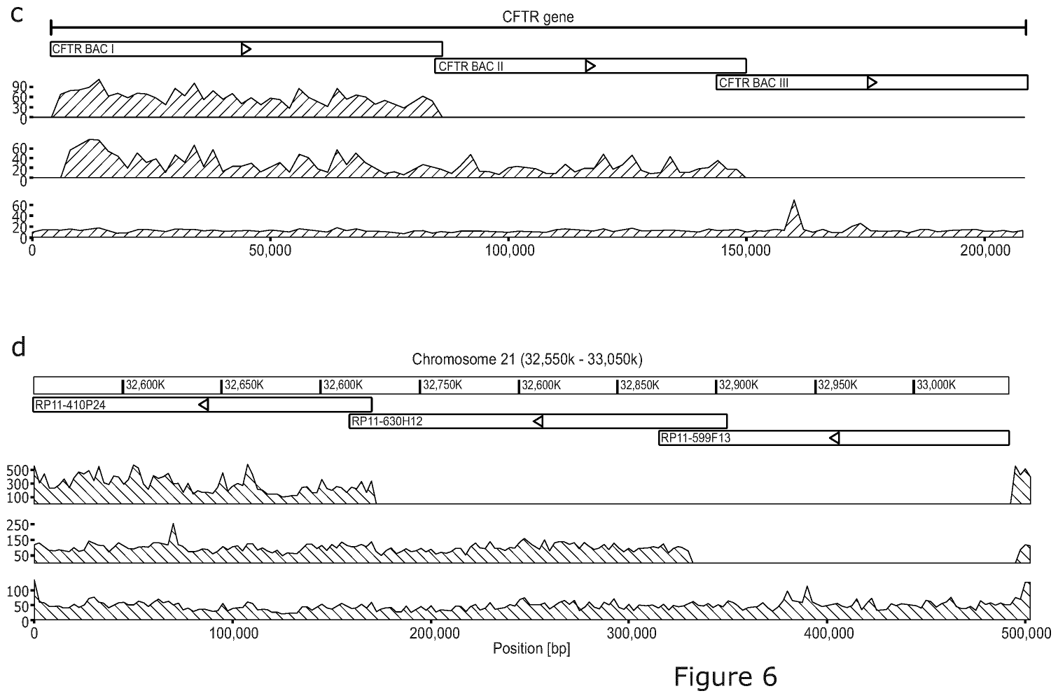

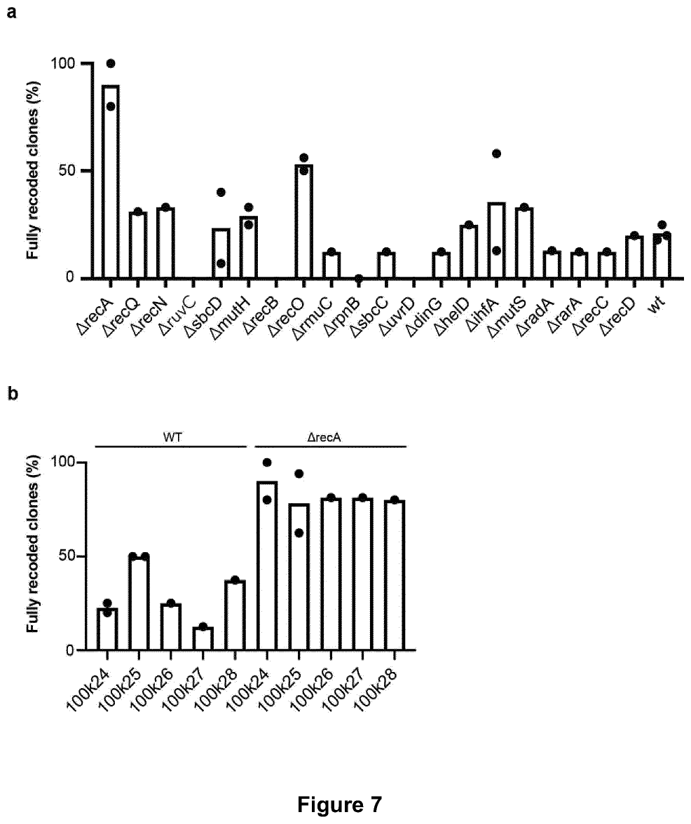

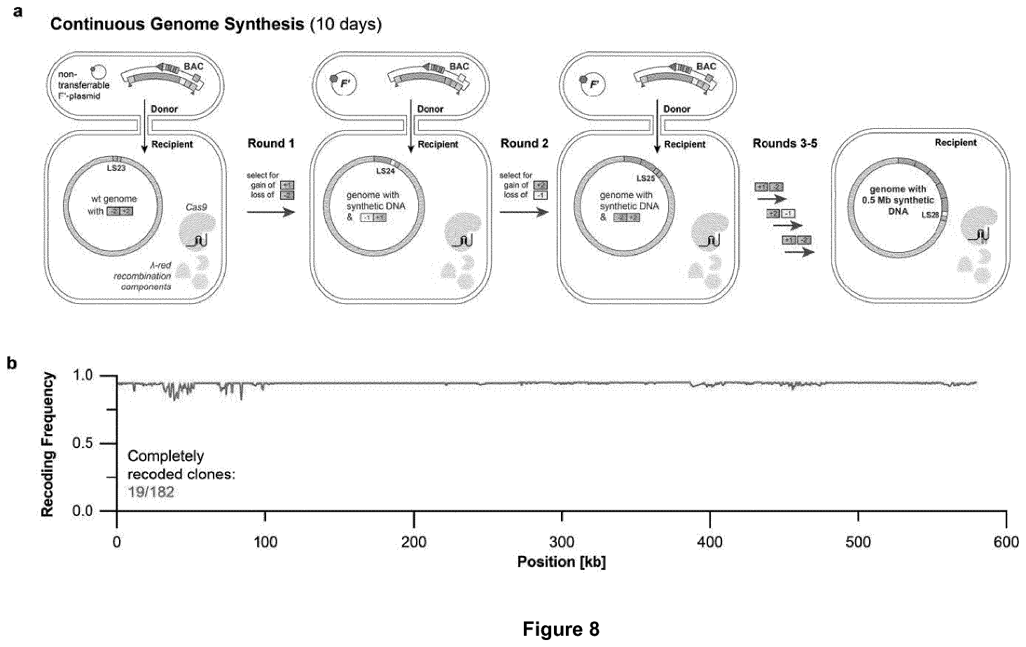

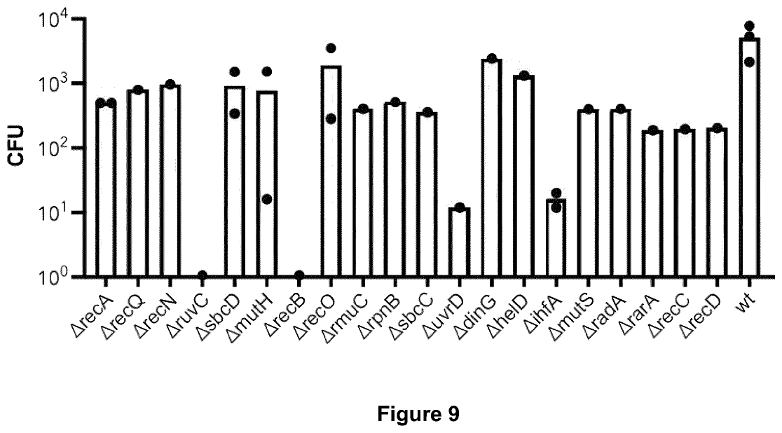

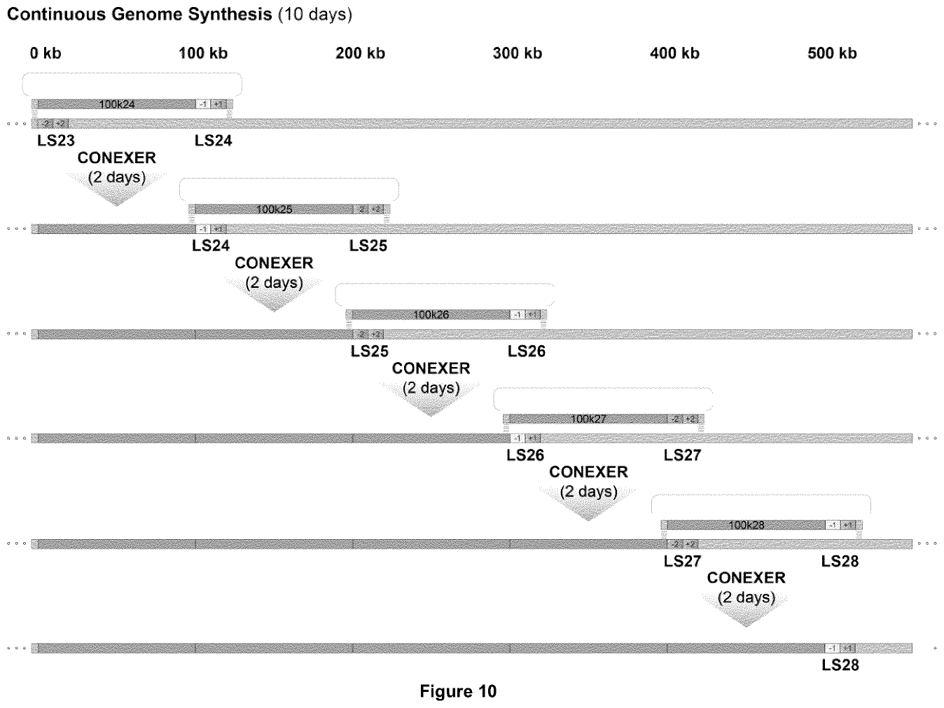

The present inventors hypothesised that, if sequences within the episomal replicon backbone, rather than the insert, could be used to direct excision of the insert from the episomal replicon then the same pair of spacers—‘universal spacers’—could be used to perform REXER at any target locus with a given episomal replicon backbone. This would massively simplify the introduction of synthetic DNA into a target nucleic acid, such as an E. coli genome, and would enable accelerated methods for large-scale genome engineering and whole genome synthesis. However, prior methods make use of spacers for Cas9 that cannot direct the precise cleavage of the junction between the episomal replicon backbone and the insert unless they specifically bind within the insert. Indeed, the arrangement of spacers that bind within the backbone and minimize the distance of the cleavage site from the junction between the backbone and the insert leads to the excision of an insert flanked by 6 base pairs of the backbone on each end (FIG. 1c). These 6 bp sequences are not commonly homologous to the region of the target nucleic acid flanking the regions complementary to the insert. It was therefore unclear whether a derivative of REXER that used universal spacers would work and whether it would lead to undesired insertions or deletions.

As demonstrated herein, the inventors provide universal spacers and demonstrate that these spacers can be used for scarless integration of synthetic DNA into a target nucleic acid. Moreover, the inventors develop an accelerated protocol for replacing genomic DNA with synthetic DNA that enables 100 kb of synthetic DNA to be introduced into a cell's genome in a single day. This approach builds upon the REXER approach that is disclosed in Wang et al. (Defining synonymous codon compression schemes by genome recoding, Nature. 2016 Nov. 3; 539(7627): 59-64. doi:10.1038/nature20124) and WO 2018/020248 (each of which is incorporated by reference). In the Examples, the inventors develop universal spacers for the two BAC backbones used for REXER-based whole genome synthesis.

These experiments reveal that the presence of short non-homologous ends on the excised synthetic DNA do not impede recombination and integration efficiency, and scarless integration is still achieved. Without being bound to a particular theory, the inventors suggest that the non-homologous ends of the DNA in the BAC may be removed by exonucleases prior to recombination, or by flap endonucleases such as EcoIX during recombination, similar to the mechanism described for FEN1 in eukaryotes. As such, the inventors have discovered that the site of the cut need not be precisely at the junction between the donor nucleic acid and the backbone of the episomal replicon.

This discovery is important because recognition of sequences that flank both sides of the actual cut site is a requirement of many RNA-guided DNA endonucleases. As such, in order to cut at the junction precisely, the RNA-guided DNA endonuclease is required to recognise part of the backbone of the episomal replicon and part of the donor nucleic acid. Now that the inventors have surprisingly discovered that the excised donor nucleic acid may tolerate regions of the backbone sequence without affecting recombination, this allows for “excision sites”, i.e. the combination of the actual cut site and the required flanking sequences, to be positioned wholly within the backbone sequence. Thus, the complexity of the process is reduced because the RNA-guided DNA endonuclease is not required to recognise any of the donor nucleic acid sequence, which will vary between rounds of the method.

Thus, in an embodiment of the invention, there is provided a method of introducing a sequence of interest into a target nucleic acid, the method comprising
a) providing a host cell
   said host cell comprising an episomal replicon,
said episomal replicon comprising a backbone sequence and a donor nucleic acid sequence,
wherein said donor nucleic acid sequence comprises in order: 5′—homologous recombination sequence 1—sequence of interest—homologous recombination sequence 2—3′,
wherein the backbone sequence comprises a first excision site positioned adjacent to homologous recombination sequence 1 and a second excision site positioned adjacent to homologous recombination sequence 2, said host cell further comprising a target nucleic acid;
b) providing helper protein(s) capable of supporting nucleic acid recombination in said host cell;
c) providing an RNA-guided DNA endonuclease;
d) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
e) inducing excision of said donor nucleic acid sequence by the RNA-guided DNA endonuclease; and
f) incubating to allow recombination between the excised donor nucleic acid and said target nucleic acid.

The steps a), b), c), and d) need not be performed in order and need not be performed as distinct steps. For instance, the RNA-guided DNA endonuclease may be provided before the provision of the helper protein(s) capable of supporting nucleic acid recombination in said host cell. The endonuclease and associated RNAs may be provided separately, at different times, and in any order. However, all components required for excision, such as the endonuclease and the associated RNAs, are provided before the induction of excision. In addition, all helper protein(s) capable of supporting nucleic acid recombination are provided before the incubation to allow recombination. Steps a), b), c), and d) may be performed simultaneously.

The episomal replicon comprises a backbone sequence and a donor nucleic acid sequence, and the backbone sequence does not overlap with the donor nucleic acid sequence. As such, a constant backbone sequence may be used for the introduction of multiple different donor nucleic acid sequences. In embodiments where multiple donor nucleic acids are introduced into a target by iterating the methods of the invention, more than one type of backbone sequence may be used. For instance, backbones comprising different markers, such as selection markers, may be used such that the successful introduction of the sequence of interest can be identified at each step.

The first and second excision sites allow for cleavage of the episomal replicon to excise the donor nucleic acid sequence. The excision sites provide all nucleic acid sequence required for recognition and cleavage by the endonuclease, and so no part of homologous recombination sequence 1 or homologous recombination sequence 2 is recognised in order for cleavage to take place. This is in contrast to the prior art (FIG. 1b) wherein homologous recombination sequence 1 and homologous recombination sequence 2 provide sequences that are recognised to enable cleavage.

The excision sites are adjacent to the homologous recombination sequences of the donor nucleic acid. Preferably each excision site is contiguous with the homologous recombination sequence. In such embodiments, there are no base pairs in between the sequences required for excision and the homologous recombination sequence. In other embodiments 1, 2, 3, or 4 base pairs of intervening sequence may be tolerated.

Any backbone sequence between the site at which the episomal replicon is cleaved and the homologous recombination sequence will be present as a part of the excised nucleic acid. As such, the excised nucleic acid may comprise: a first portion of the backbone sequence, the donor nucleic acid, and a second portion of the backbone sequence. Thus, in an embodiment, each terminus of the excised nucleic acid comprises nucleic acid sequence derived from the backbone sequence.

The first and second portion of backbone sequence may be 10, 9, 8, 7, 6 or fewer base pairs in length. In a particular embodiment, the first and/or second portion of backbone sequence is 6 base pairs in length. This embodiment is particularly relevant where the cleavage is performed by Cas9, which may cleave 3 base pairs upstream of a 3 base pair PAM.

The methods of the invention comprise the provision of a first RNA molecule that contributes to directing the RNA-guided DNA endonuclease to recognise the first excision site and a second RNA molecule that contributes to directing the RNA-guided DNA endonuclease to recognise the second excision site. The first and second RNA molecules are specific for regions of the episomal replicon that are wholly contained within the backbone sequence. The first and second RNA molecules do not recognise sequences within the donor nucleic acid sequence or within the homologous recombination sequences. The first and second RNA molecules may be of the same sequence or may be of different sequences.

An example of an RNA-guided DNA endonuclease is a CRISPR-Cas nuclease. In such embodiments, the first RNA molecule may comprise a spacer specific for the first excision site and the second RNA molecule may comprise a spacer specific for the second excision site. As discussed herein, the spacers of the first and second RNA molecules are specific for regions of the episomal replicon that are wholly contained within the backbone sequence. The regions of the episomal replicon for which the spacers are specific may be referred to as protospacers. As such, the first excision site includes the entire protospacer cognate for the spacer of the first RNA molecule and the second excision site includes the entire protospacer cognate for the spacer of the second RNA molecule. The spacers of the first and second RNA molecules do not recognise sequences within the donor nucleic acid sequence or within the homologous recombination sequences. The spacers of the first and second RNA molecules may be of the same sequence or may be of different sequences.

Thus, in embodiments where the cleavage is performed by a CRISPR-Cas nuclease, the excision sites comprise the protospacers cognate for the spacer RNAs. The excision sites also comprise the PAM.

In a particular embodiment, the first RNA molecule and the second RNA molecule are encoded by the episomal replicon and are part of the backbone sequence. In REXER the RNA molecules encode spacers that are specific for the homologous regions of the donor nucleic acid sequence, and should not be encoded by the backbone of the episomal replicon because the spacer sequences would need to vary depending on the donor nucleic acid sequence to be introduced. The present invention is not so limited and, hence, the present methods may be accelerated because the RNA molecules for directing excision may be encoded by the episomal replicon that also comprises the donor nucleic acid sequence.

This is particularly advantageous for iterative methods wherein more than one type of backbone sequence is used because the relevant spacers may be encoded directly by each backbone.

In some embodiments, the helper protein(s) capable of supporting nucleic acid recombination and/or the at least one endonuclease that is capable of cleaving the first and/or second excision site are encoded on the episomal replicon. In some embodiments, the helper protein(s) capable of supporting nucleic acid recombination and/or the at least one endonuclease that is capable of cleaving the first and/or second excision site are encoded on a separate episomal replicon, such as a plasmid. This separate episomal replicon may be known as a helper episomal replicon or a helper plasmid.

In a particular embodiment, there is provided a method comprising:
1) providing a host cell
   said host cell comprising an episomal replicon, such as a BAC,
said episomal replicon comprising a backbone sequence and a donor nucleic acid sequence,
wherein said donor nucleic acid sequence comprises in order: 5′—homologous recombination sequence 1—sequence of interest—homologous recombination sequence 2—3′,
wherein the backbone sequence comprises a first excision site positioned adjacent to the homologous recombination sequence 1 and a second excision site positioned adjacent to the homologous recombination sequence 2, wherein the first excision site comprises a first protospacer and the second excision site comprises a second protospacer, said host cell further comprising a target nucleic acid, for instance the genome of the host cell;
2) providing helper protein(s) capable of supporting nucleic acid recombination in said host cell;
3) providing a CRISPR-Cas nuclease, such as a Cas9 nuclease;
4) providing a first RNA molecule comprising a spacer specific for the first protospacer, and a second RNA molecule comprising a spacer specific for the second protospacer;
5) inducing excision of said donor nucleic acid sequence by the CRISPR-Cas nuclease; and
6) incubating to allow recombination between the excised donor nucleic acid and said target nucleic acid.

The steps 1), 2), 3), and 4) need not be performed in order and need not be performed as distinct steps. For instance, the CRISPR-Cas nuclease may be provided before the provision of the helper protein(s) capable of supporting nucleic acid recombination in said host cell. In addition, the CRISPR-Cas nuclease and the first and second RNA molecules may be provided separately, at different times, and in any order. However, all components required for excision are provided before the induction of excision. In addition, all helper protein(s) capable of supporting nucleic acid recombination are provided before the incubation to allow recombination. Steps 1), 2), 3), and 4) may be performed simultaneously.

The episomal replicon may be provided to the host cell by conjugation. Thus, the methods may further comprise the step of delivering the episomal replicon to the host cell by conjugative transfer. The episomal replicon, such as a BAC, may be included in a donor cell for transfer. The episomal replicon may comprise an origin of tranfer (oriT). The donor cell may comprise a non-transferable F′ plasmid.

The methods of the invention are particularly suitable for iteration in order to assemble large synthetic nucleic acid sequences. For instance, for the construction of artificial genomes. Thus, in an aspect of the invention, there is provided a method of assembling a nucleic acid sequence, the method comprising:
(i) performing the steps of any of the methods of the invention to introduce a first donor nucleic acid sequence into a first target nucleic acid in order to create a second target nucleic acid; and
(ii) performing the steps of any of the methods of the invention to introduce a second donor nucleic acid sequence into the second target nucleic acid in order to create a third target nucleic acid.

Parts (i) and (ii) may be iterated multiple times. This allows the introduction of a first donor nucleic acid, a second donor nucleic acid, a third donor nucleic acid, and potentially further donor nucleic acids. When the technique is iterated the product of one round of the method of the invention may act as a target nucleic acid sequence for the next round of nucleic acid introduction.

The first RNA molecules may be of the same sequence during each iteration of the method of the invention and/or the second RNA molecules may be of the same sequence during each iteration of the method of the invention. Alternatively, a first pair of RNA molecules and a second pair of RNA molecules may be used in an alternating manner during iterations of the invention, such that the first pair is used for every odd numbered iteration and the second pair is used for every even numbered iteration. The first pair of RNA molecules may be of the same sequence as the second pair of RNA molecules. The first pair of RNA molecules may comprise one RNA molecule that is the same as an RNA molecule in the second pair, and one RNA molecule that differs in sequence from an RNA molecule in the second pair. The first pair of RNA molecules may each differ in sequence from each of the RNA molecules of the second pair.

In other embodiments, further pairs of RNA molecules, such as a third pair, may be used as part of a pattern of iterations. Thus, the methods may further comprise iterating parts (i), (ii), and (iii), wherein part (iii) comprises performing the steps of any of the methods of the invention to introduce a third donor nucleic acid sequence into the third target nucleic acid in order to create a fourth target nucleic acid, and wherein part (iii) comprises the use of a third pair of RNA molecules. This pattern may be extended as desired.

The backbone sequence of the episomal replicon may be different during part (i) and during part (ii). For instance, the episomal replicon may comprise a marker or markers to allow identification or selection of the successful introduction of the sequence of interest. In order to allow rounds of nucleic acid introduction that include identification or selection, the episomal replicon of part (i) may comprise a first marker or set of markers and the episomal replicon of part (ii) may comprise a second marker or set of markers. In embodiments where parts (i) and (ii) are iterated, this may mean that a first marker or set of markers is used for every odd numbered selection and a second marker or set of markers is used for every even numbered selection. In other embodiments, further markers, such as a third marker or set of markers, may be used as part of a pattern of iterations. To allow for selection, the marker or markers for each round of nucleic acid introduction should be different from the marker or markers used in the previous round.

The backbone sequence of the episomal replicon during part (i) may be a first backbone sequence comprising a first marker or set of markers and may encode a first pair of RNA molecules as described herein. The backbone sequence of the episomal replicon during part (ii) may be a second backbone sequence comprising a second marker or set of markers and may encode a second pair of RNA molecules as described herein. This pattern may be maintained during iterations such that the first backbone sequence is present during every odd-numbered iteration and the second backbone sequence is present during every even-numbered iteration. The pattern of iterations may also include further backbone sequences, such as a third backbone sequence, comprising a further marker or set of markers and encoding further pairs of RNA molecules. The first marker or set of markers and the second marker or set of markers are different from each other, to allow selection of each successful nucleic acid introduction during the rounds of recombination. The RNA molecules encoded by each backbone sequence allow cleavage of the encoding backbone.

In a further aspect, the principles established for CONEXER can be extended to realize the scarless assembly and cloning, through iterative insertion, of megabases of DNA in episomes in E. coli. The invention thus concerns an assembly episomal replicon in which to iteratively insert and assemble DNA (FIG. 6a). In embodiments, this replicon comprises approximately 50 bp of sequence homologous to one end of the next sequence to be inserted (HR1); this is immediately followed by a positive and negative selection cassette and a universal homology region (uHR), which is complementary to the other end of the sequence to be inserted. Longer homologies, as long as multiple 10s of kb of sequence, may also be employed.

The invention also provides donor episomal replicons with the CONEXER backbone, containing universal spacers and oriT (FIG. 6a). In the donor replicons, HR1 is within one end of the next DNA sequence to be inserted into the recipient BAC; this DNA sequence is followed by a distinct positive and negative selection cassette and a universal homology region. Each step of assembly (FIG. 6a,b) proceeds by conjugation of the donor replicon to recipient cells containing the assembly replicon, nuclease-mediated excision of the sequence from HR1 to uHR from the donor replicon, and recombination mediated insertion of this sequence into the assembly replicon. Selection for loss of the negative selection markers on the assembly replicon, and gain of the positive marker from the sequence excised from the donor replicon selects for cells containing the assembly replicon with the correct insertion. Cells containing the new assembly replicon provide the input for the next step of insertion. This approach was named BAC stepwise insertion synthesis (BASIS).

Therefore, in a further aspect of the invention, there is provided a method for constructing an episomal replicon comprising a plurality of assembly steps, wherein step n comprises the steps of:
a) providing a donor episomal replicon, said replicon comprising:
   a backbone, said backbone comprising universal spacer sequences, a first homology region HRn which is specific for an integration step n, and a second, universal, homology region uHR, a first excision site positioned adjacent to HRn and a second excision site positioned adjacent to uHR;
   a donor nucleic acid DNAn, said donor nucleic acid comprising a homology region HRn+1, specific for an assembly step n+1;
   a double selection cassette, comprising positive and negative selection markers;
b) providing a host cell comprising an assembly episomal replicon comprising a double selection cassette comprising positive and negative selection markers, flanked by HRn and uHR, the double selection cassette in the assembly replicon comprising different markers to the selection cassette in the donor replicon;
c) providing helper protein(s) capable of supporting nucleic acid recombination in said host cell;
c) providing an RNA-guided DNA endonuclease;
d) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
e) inducing excision of said donor nucleic acid sequence DNAn by the RNA-guided DNA endonuclease in the host cell; and
f) incubating to allow recombination between the excised donor nucleic acid and said assembly replicon to form a second assembly replicon, which comprises the nucleic acid DNAn.

The assembly replicon carrying the donor nucleic acid can in turn be used as an assembly replicon in a second step, in which a second donor replicon comprising homology regions HIRn+1 and uHR and a second donor nucleic acid DNAn+1 is employed to introduce a second donor nucleic acid into the assembly replicon generated in the first step.

Alternating positive and negative selection marker sets allow an infinite number of steps to be performed iteratively, assembling an episomal replicon of any desired size. Preferably, the number of steps performed may be at least 2, and 100 or less; 50 or less; 25 or less; and most preferably about 10, 9, 8, 7, 6 or 5.

The size of the replicon which is assembled is preferably between 1 and 100 Mb. Preferably, it is 2 to 50 Mb, 3 to 25 Mb, 4 to 15 Mb, or 5 to 10 Mb.

Thus, in an embodiment, the invention comprises performing the method of the above aspect of the invention, further comprising the steps of introducing into the host cell a further donor episomal replicon comprising a donor nucleic acid DNAn+1, and inducing excision of said donor nucleic acid sequence DNAn by the RNA-guided DNA endonuclease in the host cell; and incubating to allow recombination between the excised donor nucleic acid DNAn+1 and said assembly replicon to form a second assembly replicon, which comprises the nucleic acid DNAn and nucleic acid DNAn+1.

The steps of this embodiment of the invention may be performed iteratively, inserting donor nucleic acids DNAn+2, n+3, n+4 etc into the assembly episomal replicon.

BASIS can be used to generate episomes or other DNA vectors or segments which are useful for continuous genome synthesis (CGS). BASIS can also be itself applied continuously, without sequencing steps, for the continuous production of artificial DNA, whether episomes, genomes, bacterial or other genes and DNA to processes for continuous genome synthesis (CGS). The inventors first demonstrated the assembly of the 208 Kbp human Cystic Fibrosis Transmembrane regulator gene by BASIS. The CFTR gene was assembled in three steps of BASIS with donor BACS that contained approximately 70 Kbp fragments of the gene.

BASIS can be used to assemble large sections of human genomic DNA, which includes exonic, intronic and intergenic regions, into a single episome.

In order to allow rapid iteration of CONEXER by directly using an un-sequenced pool of clones from one CONEXER as the input for the next CONEXER, the removal of sequencing steps to validate each clone is desirable. Factors that substantially increase the fraction of clones in which the genomic DNA has been completely replaced with synthetic DNA in a single step of CONEXER were therefore investigated.

Both recA and recO have been identified as factors that increase the fraction of clones with fully synthetic sequence (FIG. 7a, FIG. 9). Deletion of recA (ΔrecA) increased the fraction of fully synthetic DNA from 20% to approximately 80% for 100k 24 (FIG. 7a). Similar dramatic increases were observed across several other 100 Kbp regions, underscoring the generality of these observations (FIG. 7b).

Continuous genome synthesis can be performed by directly using the output from one round of CONEXER—without identifying an individual, fully recoded clone by sequencing—as the input for the next round of CONEXER.

Accordingly, the host cell used in aspects of the present invention is advantageously lacking competent recA and/or recO. Preferably, the host cell lacks recA (ΔrecA).

### Episomal Replicons

Embodiments of the invention comprise an episomal replicon comprising a donor nucleic acid sequence. The donor nucleic acid may be DNA.

The term “episome” has its ordinary meaning in the art, for example any accessory extrachromosomal replicating genetic element that can exist either autonomously or can become integrated with the chromosome.

An episomal replicon is an episomal nucleic acid which possesses its own origin of replication capable of functioning within said host cell.

The episomal replicon may be a plasmid. A plasmid means a small circular nucleic acid (usually DNA, most usually double-stranded DNA) molecule. A plasmid within a cell is physically separated from any chromosomal nucleic acid such as DNA and can replicate independently. Considering plasmids, “small” means they are typically no bigger than 10 kb. Suitably a plasmid useful in the invention has the following genetic elements: an origin of replication cognate for the host cell; and at least one selection marker.

The episomal replicon may be a BAC. The BAC may comprise the following genetic elements: an origin of replication cognate for the host cell; and at least one selection marker.

BACs and plasmids differ from each other by their replication origin. A BAC has a special replication origin which typically makes the BAC a single copy in each cell and helps the BAC to maintain a bigger size (up to several hundred kb). Plasmids have a plasmid replication origin which typically makes the plasmid multiple copies (ranging from a few copies to a few hundred copies per cell) in each cell and typically of a size up to around 10 kb.

The episomal replicon may be a yeast artificial chromosome (YAC). The YAC may comprise the following genetic elements: an origin of replication cognate for the host cell; and at least one selection marker.

Multiple origins of replication active in the same cell on the same single nucleic acid are not usually desirable. This is especially true for example when a multicopy episomal nucleic acid such as a plasmid is carrying the donor nucleic acid—in this scenario it is clearly not desirable to incorporate the plasmid origin of replication into (for example) a BAC or into the host genome. Thus, suitably said excised linear donor nucleic acid does not comprise an origin of replication. Suitably the target nucleic acid sequence comprises an origin of replication.

Suitably the origin of replication on the episomal replicon comprising the donor sequence must match with host, e.g. all prokaryotic. Suitably the origins of replication on the episomal replicon comprising the target and on the episomal replicon comprising donor sequence must match with host, e.g. all prokaryotic.

Suitably the episomal replicon comprising the donor sequence comprises a prokaryotic origin of replication. Suitably the replicon comprising the target sequence comprises a prokaryotic origin of replication. Suitably the replicon comprising the target sequence is an episomal replicon and comprises a prokaryotic origin of replication. Suitably the host cell is prokaryotic. Suitably the synthetic genome is a synthetic prokaryotic genome.

### Target Nucleic Acid

The target nucleic acid may be any suitable for the introduction of the donor nucleic acid sequence. In particular, the target nucleic acid may be a DNA molecule suitable for the introduction of a donor DNA molecule.

The target nucleic acid may comprise homologous recombination sequence 1 and homologous recombination sequence 2. The target nucleic acid may comprise a selection marker or selection markers, which may be flanked by the homologous recombination sequences. For instance, the target nucleic acid may comprise a negative selection marker.

The target nucleic acid may be a region of a nucleic acid that also possess its own origin of replication capable of functioning within the host cell. The target nucleic acid may be a plasmid. The target nucleic acid may be a BAC. The target nucleic acid may be a YAC. In a particular embodiment, the target nucleic acid is the genome of the host cell.

When the invention is applied to a genome, suitably the genome is a non-human genome, suitably a non-mammalian genome. Suitably the genome is a prokaryotic genome, suitably a bacterial genome. In particular, the genome may be an E. Coli genome.

In a particular embodiment, the episomal replicon comprising a donor DNA molecule is a BAC and the target nucleic acid is the genome of an E. Coli cell.

### Homologous Recombination

In theory any nucleotide sequence can be chosen as the site for homologous recombination sequences.

The nucleotide sequence for homologous recombination may be unique. For instance, the nucleotide sequence for homologous recombination may be unique within the target sequence into which the donor sequence is being recombined. In other examples, homologous recombination sequence 1 and/or homologous recombination sequence 2 may be unique within the target sequence into which the donor sequence is being recombined.

Alternatively, homologous recombination sequence 1 and/or homologous recombination sequence 2 may be not unique within the target sequence into which the donor sequence is being recombined. In such examples, selection may be used to identify the successful introduction of the sequence of interest into the desired site. For instance, off-target integration may not result in the removal or disruption of a negative selection marker, or off-target integration may not repair double-strand breaks induced at the site of introduction.

Suitably the sequence for homologous recombination is non-repetitive.

Suitably the sequence for homologous recombination is at least 30 nucleotides long. Homologous recombination sequences as short as 30 nucleotides may lead to a low efficiency; thus for high efficiency suitably the homologous recombination sequence is at least 40 nucleotides in length, suitably at least 50 nucleotides, suitably 50 to 100 nucleotides, most suitably 50 to 65 nucleotides.

The sequence for homologous recombination is selected on the target sequence and introduced into the donor sequence. Therefore, the homologous recombination sequence 1 (HR1) and homologous recombination sequence 2 (HR2) on the donor sequence show 100% sequence identity to the HR1 and HR2 on the target sequence.

Use of lambda Red recombination permits short nucleotide sequences to be used for homologous recombination, as outlined above. Other recombination support systems may be used. For example, the RecBCD system might be used. When the RecBCD system is used, suitably the step of “providing helper protein(s) capable of supporting nucleic acid recombination in said host cell” consists of inducing or permitting expression of the RecBCD system within the host cell.

When using the RecBCD system or other recombination support systems, the skilled operator will pay attention to the requirements of those systems on the sequences selected for homologous recombination. For example, the RecBCD system may require longer homologous recombination sequences such as 3 to 10 kb in length.

In more detail, RecBCD is a natural E. coli recombination system consisting of three components RecB, RecC, and RecD. The three subunits make up an ATP-dependent helicase/nuclease complex that is essential for both homologous recombination during the course of transduction and conjugation as well as in repair of double-strand breaks in E. coli. Studies in which double strand breaks are induced in vivo in E. coli DNA show that double-strand break repair (DSBR) can proceed via one of two recombination pathways. Both pathways require RecBCD and RecA, but one depends on the resolvase enzyme, RuvABC, while the other does not and instead relies on RecG. The recB and recD genes form an operon while recC is situated nearby but has its own promoter. The three gene products form a heterotrimer which is also known as Exonuclease V. In case any further guidance is needed, details can be found in the publicly available EcoCyc database under ‘RecBCD’, for example for the K12-MG-1655 strain of E. coli (Keseler et al. (2013), “EcoCyc: fusing model organism databases with systems biology”, Nucleic Acids Research 41: D605-12).

Thus, to support recombination according to this embodiment, at least RecBCD should be expressed in the host cell.

It may be that RecA is also required; thus, more suitably to support recombination according to this embodiment, at least RecBCD and RecA should be expressed in the host cell. Most suitably to support recombination according to this embodiment, RecBCD and RecA should be expressed in the host cell.

Another alternative to the lambda red system is the RecET system. RecE and RecT are E. coli genes of phage origin. RecE mimics lambda red alpha, and RecT mimics lambda red beta (Muyrers, J. P., Zhang, Y., Buchholz, F. & Stewart, A. F. RecE/RecT and Redalpha/Redbeta initiate double-stranded break repair by specifically interacting with their respective partners. Genes Dev. 14, 1971-1982 (2000)). The RecET combination performs comparatively to lambda red alpha/beta combination. Lambda red alpha and beta are the actual components that carry out recombination, while lambda red gamma is an inhibitor of the RecBCD system.

Suitably recombination support is provided via the lambda red system, for example from the commercially available pRed/ET plasmid from Gene Bridges (“Quick & Easy E. coli Gene Deletion Kit” from Gene Bridges GmbH, Im Neuenheimer Feld 584, 69120 Heidelberg, Germany.).

This system in this setup is first described in Datsenko et al 2000 (Datsenko K. A. & Wanner, B. L. One-step inactivation of chromosomal genes in Escherichia coli K-12 using PCR products. Proc. Natl. Acad. Sci. U.S.A. 97, 6640-6645 (2000)), which is hereby incorporated herein by reference specifically for details of the Lambda Red system.

The inventors teach that the pRed/ET plasmid is based on the pKD46 plasmid in Datsenko et al 2000 (as judged by sequence identity), and therefore the pKD46 plasmid may be used as a template to perform PCR for the construction of lambda red system.

When said helper protein(s) capable of supporting nucleic acid recombination comprise lambda Red proteins, suitably the following proteins are expressed in said host cell:

TABLE 1

| Lambda | | | |
|---|---|---|---|
| Red | Essential | | Exemplary sequence/accession |
| protein | ? | Function/notes | number |
| alpha | essential | SEQ ID NO: 1 | ATGACACCGGACATTATCCTGCAGCGTACCGGGATCGA |
| | | Lambda red | TGTGAGAGCTGTCGAACAGGGGGATGATGCGTGGCACA |
| | | recombination | AATTACGGCTCGGCGTCATCACCGCTTCAGAAGTTCAC |
| | | | AACGTGATAGCAAAACCCCGCTCCGGAAAGAAGTGGCC |
| | | | TGACATGAAAATGTCCTACTTTCACACCCTGCTTGCTG |
| | | | AGGTTTGCACCGGTGTGGCTCCGGAAGTTAACGCTAAA |
| | | | GCACTGGCCTGGGGAAAACAGTACGAGAACGACGCCAG |
| | | | AACCCTGTTTGAATTCACTTCCGGCGTGAATGTTACTG |
| | | | AATCCCCGATCATCTATCGCGACGAAAGTATGCGTACC |
| | | | GCCTGCTCTCCCGATGGTTTATGCAGTGACGGCAACGG |
| | | | CCTTGAACTGAAATGCCCGTTTACCTcccgggATTTC |
| | | | ATGAAGTTCCGGCTCGGTGGTTTCGAGGCCATAAAGT |
| | | | CAGCTTACATGGCCCAGGTGCAGTACAGCATGTGGGT |
| | | | GACGCGAAAAAATGCCTGGTACTTTGCCAACTATGAC |
| | | | CCGCGTATGAAGCGTGAAGGCCTGCATTATGTCGTGA |
| | | | TTGAGCGGGATGAAAAGTACATGGCGAGTTTTGACGA |
| | | | GATCGTGCCGGAGTTCATCGAAAAAATGGACGAGGCA |
| | | | CTGGCTGAAATTGGTTTTGTATTTGGGGAGCAATGGC |
| | | | GATAG |
| beta | essential | SEQ ID NO: 2 | ATGAGTACTGCACTCGCAACGCTGGCTGGGAAGCTGG |
| | | Lambda red | CTGAACGTGTCGGCATGGATTCTGTCGACCCACAGGA |
| | | recombination | ACTGATCACCACTCTTCGCCAGACGGCATTTAAAGGT |
| | | | GATGCCAGCGATGCGCAGTTCATCGCATTACTGATCG |
| | | | TTGCCAACCAGTACGGCCTTAATCCGTGGACGAAAGA |
| | | | AATTTACGCCTTTCCTGATAAGCAGAATGGCATCGTT |
| | | | CCGGTGGTGGGCGTTGATGGCTGGTCCCGCATCATCA |
| | | | ATGAAAACCAGCAGTTTGATGGCATGGACTTTGAGCA |
| | | | GGACAATGAATCCtgtacaTGCCGGATTTACCGCAAG |
| | | | GACCGTAATCATCCGATCTGCGTTACCGAATGGATGG |
| | | | ATGAATGCCGCCGCGAACCATTCAAAACTCGCGAAGG |
| | | | CAGAGAAATCACGGGGCCGTGGCAGTCGCATCCCAAA |
| | | | CGGATGTTACGTCATAAAGCCATGATTCAGTGTGCCC |
| | | | GTCTGGCCTTCGGATTTGCTGGTATCTATGACAAGGA |
| | | | TGAAGCCGAGCGCATTGTCGAAAATACTGCATACACT |
| | | | GCAGAACGTCAGCCGGAACGCGACATCACTCCGGTTA |
| | | | ACGATGAAACCATGCAGGAGATTAACACTCTGCTGAT |
| | | | CGCCCTGGATAAAACATGGGATGACGACTTATTGCCG |
| | | | CTCTGTTCCCAGATATTTCGCCGCGACATTCGTGCAT |
| | | | CGTCAGAACTGACACAGGCCGAAGCAGTAAAAGCTCT |
| | | | TGGATTCCTGAAACAGAAAGCCGCAGAGCAGAAGGTG |
| | | | GCAGCATGA |
| gamma | | SEQ ID NO: 3 | ATGGATATTAATACTGAAACTGAGATCAAGCAAAAGC |
| | | inhibiting | ATTCACTAACCCCCTTTCCTGTTTTCCTAATCAGCCC |
| | | RecBCD | GGCATTTCGCGGGCGATATTTTCACAGCTATTTCAGG |
| | | | AGTTCAGCCATGAACGCTTATTACATTCAGGATCGTC |
| | | | TTGAGGCTCAGAGCTGGGCGCGTCACTACCAGCAGCT |
| | | | CGCCCGTGAAGAGAAAGAGGCAGAACTGGCAGACGAC |
| | | | ATGGAAAAAGGCCTGCCCCAGCACCTGTTTGAATCGC |
| | | | TATGCATCGATCATTTGCAACGCCACGGGGCCAGCAA |
| | | | AAAATCCATTACCCGTGCGTTTGATGACGATGTTGAG |
| | | | TTTCAGGAGCGCATGGCAGAACACATCCGGTACATGG |
| | | | TTGAAACCATTGCTCACCACCAGGTTGATATTGATTC |
| | | | AGAGGTATAA |

### Homologous Recombination Sequences

In order to choose a homologous recombination sequence, the following steps may be used:
Choose 50 to 100 nucleotides in the desired position in the sequence of the nucleic acid being altered (target nucleic acid) such as a bacterial genome or plasmid backbone.
Perform a BLAST search of the chosen sequence against the target nucleic acid.
Consider the E-value for the chosen sequence compared to the closest match in the BLAST search—typically an E-value compared to an undesired target site elsewhere in the target nucleic acid of greater than 10⁻²⁰ would be too high; if this is discovered then suitably an alternative homologous recombination sequence is selected.

Suitably standard BLAST tool is used to calculate the E-value for homologous recombination (HR) sequences. One such online tool is at http://biocyc.org/ECOLI/blast.html. Suitably the focus is on how unique a given HR sequence is as judged by E-value. Suitably it is not necessary to consider/calculate affinity. In principle any sequence that can work with classical recombination, is going to work better with the invention.

In more detail, if HR sequences can work with classical recombination, they are going to work better in the invention. Suitably the HR sequences for the invention are selected following the exact principle and requirement as for classical recombination using lambda red system. For example, the inventors typically design HRs 50-70 bp in length and blast against the E. coli genome for an expected value lower than 10⁻²⁰. (E-value, a measurement of how unique a given sequence is; the lower the E-value is, the more unique the sequence is. Any suitable tool for calculation may be used, for example standard BLAST tool to calculate the E-value for HR sequences. One such online tool is at http://biocyc.org/ECOLI/blast.html.). Values lower than 10⁻²⁰ E-value are not expected to be necessary, although of course sequences with lower values are still useful in the invention.

The E-value is a measurement of how unique a given sequence is. Because classical recombination solely relies on the specificity of the homology regions, it requires a relatively stringent E-value cut off such as 10⁻²⁰. Because the methods of the invention may boost locus specificity not only by the specificity of the homology regions but also by the simultaneous loss of the negative selection marker and gain of positive selection marker, the methods can in principle tolerate less stringent E-value(s) (e.g. less stringent homology regions). However, it is practically very straightforward to generate homology regions with stringent E-value, so suitably the 10⁻²⁰ E-value cut off is used.

### Selectable Markers

Any of the methods of the invention may comprise the further step of selecting for recombinants having incorporated the donor nucleic acid into the target nucleic acid. This step would be performed after the induction step to allow recombination.

The sequence of interest may comprise a positive selectable marker. Such markers include any that would allow the identification or selection of a cell comprising the marker.

The target nucleic acid may comprise in order: 5′—homologous recombination sequence 1—negative selectable marker—homologous recombination sequence 2-3′.

Selecting for recombinants having incorporated said donor nucleic acid into said target nucleic acid may comprise selection for gain of the positive selectable marker of the donor nucleic acid and loss of the negative selectable marker of the target nucleic acid. Suitably selection for gain of the positive selectable marker of the donor nucleic acid and loss of the negative selectable marker of the target nucleic acid is carried out simultaneously. In other embodiments, the step of selecting for recombinants comprises sequential selection for the positive and negative markers, or sequential selection for the negative and positive markers.

The sequence of interest may comprise both a positive selectable marker and a negative selectable marker.

The methods of the invention may further comprise the step of:
inducing at least one double stranded break in the target nucleic acid sequence,
wherein said double stranded break is between said homologous recombination sequence 1 and said homologous recombination sequence 2.

Suitably at least two double stranded breaks are induced in the target nucleic acid sequence, wherein each said double stranded break is between said homologous recombination sequence 1 and said homologous recombination sequence 2.

The episomal replicon may comprise a negative selectable marker independent of the donor nucleic acid sequence. Suitably said method comprises the further step of selecting for loss of the episomal replicon by selecting for loss of said negative selectable marker independent of the donor nucleic acid sequence.

Some methods of the invention comprise a combinatorial selection approach involving a positive marker and loss of a negative marker. Use of this “double selection” scheme actually also helps with site specificity. For example, if a recombination event takes place at an inappropriate site, it could result in acquisition of the positive selectable marker. However, by using simultaneous selection for the positive marker and loss of the negative marker, even if the nucleic acid has been incorporated into the target nucleic acid at an inappropriate site (thereby conferring the positive marker), such molecules still would not be selected because if they have recombined into an inappropriate site they will not have simultaneously resulted in the loss of the negative marker. Therefore, as well as being a useful selection in its own right, this actually adds to the technical benefit of assisting in the site specificity by selecting not only for acquisition of the donor sequence but also simultaneous deletion of the sequence being removed/replaced.

Examples of suitable selectable markers are shown in the following table (Table 2). Furthermore, any suitable antibiotic marker may be used. Examples of such antibiotic markers include TetR, AmpR, HyR, and ErmR, which allow for a selection scheme including tetracycline, ampicillin, hygromycin, or erythromycin resistance, respectively.

| | Selection | |
|---|---|---|
| Marker | Scheme | Accession number/nucleic acid |
| sacB | Sucrose | sacB |
| | sensitivity | ATGAACATCAAAAAGTTTGCAAAACAAGCAACAGTATTAACCTTTACTACCGCACTGCTGGCAGGAGGC |
| | SEQ ID NO: 4 | GCAACTCAAGCGTTTGCGAAAGAAACGAACCAAAAGCCATATAAGGAAACATACGGCATTTCCCATATT |
| | | ACACGCCATGATATGCTGCAAATCCCTGAACAGCAAAAAAATGAAAAATATAAAGTTCCTGAGTTCGAT |
| | | TCGTCCACAATTAAAAATATCTCTTCTGCAAAAGGCCTGGACGTTTGGGACAGCTGGCCATTACAAAAC |
| | | CACTGACGGCACTGTCGCAAACTATCACGGCTACCACATCGTCTTTGCATTAGCCGGAGATCCTAAAAA |
| | | TGCGGATGACACATCGATTTACATGTTCTATCAAAAAGTCGGCGAAACTTCTATTGACAGCTGGAAAAA |
| | | CGCTGGCCGCGTCTTTAAAGACAGCGACAAATTCGATGCAAATGATTCTATCCTAAAAGACCAAACACA |
| | | AGAATGGTCAGGTTCAGCCACATTTACATCTGACGGAAAAATCCGTTTATTCTACACTGATTTCTCCGG |
| | | TAAACATTACGGCAAACAAACACTGACAACTGCACAAGTTAACGTATCAGCATCAGACAGCTCTTTGAA |
| | | CATCAACGGTGTAGAGGATTATAAATCAATCTTTGACGGTGACGGAAAAACGTATCAAAATGTACAGCA |
| | | GTTCATCGATGAAGGCAACTACAGCTCAGGCGACAACCATACGCTGAGAGATCCTCACTACGTAGAAGA |
| | | TAAAGGCCACAAATACTTAGTATTTGAAGCAAACACTGGAACTGAAGATGGCTACCAAGGCGAAGAATC |
| | | TTTATTTAACAAAGCATACTATGGCAAAAGCACATCATTCTTCCGTCAAGAAAGTCAAAAACTTCTGCA |
| | | AAGCGATAAAAAACGCACGGCTGAGTTAGCAAACGGCGCTCTCGGTATGATTGAGCTAAACGATGATTA |
| | | CACACTGAAAAAAGTGATGAAACCGCTGATTGCATCTAACACAGTAACAGATGAAATTGAACGCGCGAA |
| | | CGTCTTTAAAATGAACGGCAAATGGTACCTGTTCACTGACTCCCGCGGATCAAAAATGACGATTGACGG |
| | | CATTACGTCTAACGATATTTACATGCTTGGTTATGTTTCTAATTCTTTAACTGGCCCATACAAGCCGCT |
| | | GAACAAAACTGGCCTTGTGTTAAAAATGGATCTTGATCCTAACGATGTAACCTTTACTTACTCACACTT |
| | | CGCTGTACCTCAAGCGAAAGGAAACAATGTCGTGATTACAAGCTATATGACAAACAGAGGATTCTACGC |
| | | AGACAAACAATCAACGTTTGCGCCTAGCTTCCTGCTGAACATCAAAGGCAAGAAAACATCTGTTGTCAA |
| | | AGACAGCATCCTTGAACAAGGACAATTAACAGTTAACAAATAA |
| rpsL (S12 | Streptomycin | rps L |
| ribosomal | sensitivity | ATGGCAACAGTTAACCAGCTGGTACGCAAACCACGTGCTCGCAAAGTTGCGAAAAGCAACGTGCCTGCG |
| protein) | SEQ ID NO: 5 | CTGGAAGCATGCCCGCAAAAACGTGGCGTATGTACTCGTGTATATACTACCACTCCTAAAAAACCGAAC |
| | | TCCGCGCTGCGTAAAGTATGCCGTGTTCGTCTGACTAACGGTTTCGAAGTGACTTCCTACATCGGTGGT |
| | | GAAGGTCACAACCTGCAGGAGCACTCCGTGATCCTGATCCGTGGCGGTCGTGTTAAAGACCTCCCGGGT |
| | | GTTCGTTACCACACCGTACGTGGTGCGCTTGACTGCTCCGGCGTTAAAGACCGTAAGCAGGCTCGTTCC |
| | | AAGTATGGCGTGAAGCGTCCTAAGGCTTAA |
| PheS with | P-chloro- | PheS T251A A294G |
| two point | phenylalanine | ATGTCACATCTCGCAGAACTGGTTGCCAGTGCGAAGGCGGCCATTAGCCAGGCGTCAGATGTTGCCGCG |
| mutations | SEQ ID NO: 6 | TTAGATAATGTGCGCGTCGAATATTTGGGTAAAAAAGGGCACTTAACCCTTCAGATGACGACCCTGCGT |
| | | GAGCTGCCGCCAGAAGAGCGTCCGGCAGCTGGTGCGGTTATCAACGAAGCGAAAGAGCAGGTTCAGCAG |
| | | GCGCTGAATGCGCGTAAAGCGGAACTGGAAAGCGCTGCACTGAATGCGCGTCTGGCGGCGGAAACGATT |
| | | GATGTCTCTCTGCCAGGTCGTCGCATTGAAAACGGCGGTCTGCATCCGGTTACCCGTACCATCGACCGT |
| | | ATCGAAAGTTTCTTCGGTGAGCTTGGCTTTACCGTGGCAACCGGGCCGGAAATCGAAGACGATTATCAT |
| | | AACTTCGATGCTCTGAACATTCCTGGTCACCACCCGGCGCGCGCTGACCACGACACTTTCTGGTTTGAC |
| | | ACTACCCGCCTGCTGCGTACCCAGACCTCTGGCGTACAGATCCGCACCATGAAAGCCCAGCAGCCACCG |
| | | ATTCGTATCATCGCGCCTGGCCGTGTTTATCGTAACGACTACGACCAGACTCACACGCCGATGTTCCAT |
| | | CAGATGGAAGGTCTGATTGTTGATACCAACATCAGCTTTACCAACCTGAAAGGCACGCTGCACGACTTC |
| | | CTGCGTAACTTCTTTGAGGAAGATTTGCAGATTCGCTTCCGTCCTTCCTACTTCCCGTTTGCCGAACCT |
| | | TCTGCAGAAGTGGACGTCATGGGTAAAAACGGTAAATGGCTGGAAGTGCTGGGCTGCGGGATGGTGCAT |
| | | CCGAACGTGTTGCGTAACGTTGGCATCGACCCGGAAGTTTACTCTGGTTTCGGCTTCGGGATGGGGATG |
| | | GAGCGTCTGACTATGTTGCGTTACGGCGTCACCGACCTGCGTTCATTCTTCGAAAACGATCTGCGTTTC |
| | | CTCAAACAGTTTAAATAA |
| Cm^{R} | Chloramphen- | CmR |
| | icol | ATGGAGAAAAAAATCACTGGATATACCACCGTTGATATATCCCAATGGCATCGTAAAGAACATTTTGAG |
| | resistance | GCATTTCAGTCAGTTGCTCAATGTACCTATAACCAGACCGTTCAGCTGGATATTACGGCCTTTTTAAAG |
| | SEQ ID NO: 7 | ACCGTAAAGAAAAATAAGCACAAGTTTTATCCGGCCTTTATTCACATTCTTGCCCGCCTGATGAATGCT |
| | | CATCCGGAATTCCGTATGGCAATGAAAGACGGTGAGCTGGTGATATGGGATAGTGTTCACCCTTGTTAC |
| | | ACCGTTTTCCATGAGCAAACTGAAACGTTTTCATCGCTCTGGAGTGAATACCACGACGATTTCCGGCAG |
| | | TTTCTACACATATATTCGCAAGATGTGGCGTGTTACGGTGAAAACCTGGCCTATTTCCCTAAAGGGTTT |
| | | ATTGAGAATATGTTTTTCGTCTCAGCCAATCCCTGGGTGAGTTTCACCAGTTTTGATTTAAACGTGGCC |
| | | AATATGGACAACTTCTTCGCCCCCGTTTTCACCgTGGGCAAATATTATACGCAAGGCGACAAGGTGCTG |
| | | ATGCCGCTGGCGATTCAGGTTCATCATGCCGTCTGTGATGGCTTCCATGTCGGCAGAATGCTTAATGAA |
| | | TTACAACAGTACTGCGATGAGTGGCAGGGCGGGGCGTAA |
| KanR | Kanamycin | KanR |
| | resistance | ATGATTGAACAAGATGGATTGCACGCAGGTTCTCCGGCCGCTTGGGTGGAGAGGCTATTCGGCTATGAC |
| | SEQ ID NO: 8 | TGGGCACAACAGACAATCGGCTGCTCTGATGCCGCCGTGTTCCGGCTGTCAGCGCAGGGGCGCCCGGTT |
| | | CTTTTTGTCAAGACCGACCTGTCCGGTGCCCTGAATGAACTGCAGGACGAGGCAGCGCGGCTATCGTGG |
| | | CTGGCCACGACGGGCGTTCCTTGCGCAGCTGTGCTCGACGTTGTCACTGAAGCGGGAAGGGACTGGCTG |
| | | CTATTGGGCGAAGTGCCGGGGCAGGATCTCCTGTCATCTCACCTTGCTCCTGCCGAGAAAGTATCCATC |
| | | ATGGCTGATGCAATGCGGCGGCTGCATACGCTTGATCCGGCTACCTGCCCATTCGACCACCAAGCGAAA |
| | | CATCGCATCGAGCGAGCACGTACTCGGATGGAAGCCGGTCTTGTCGATCAGGATGATCTGGACGAAGAG |
| | | CATCAGGGGCTCGCGCCAGCCGAACTGTTCGCCAGGCTCAAGGCGCGCATGCCCGACGGCGAGGATCTC |
| | | GTCGTGACCCATGGCGATGCCTGCTTGCCGAATATCATGGTGGAAAATGGCCGCTTTTCTGGATTCATC |
| | | GACTGTGGCCGGCTGGGTGTGGCGGACCGCTATCAGGACATAGCGTTGGCTACCCGTGATATTGCTGAA |
| | | GAGCTTGGCGGCGAATGGGCTGACCGCTTCCTCGTGCTTTACGGTATCGCCGCTCCCGATTCGCAGCGC |
| | | ATCGCCTTCTATCGCCTTCTTGACGAGTTCTTCTGA |

Thus, in an embodiment, the negative selectable marker is selected from the group consisting of sacB (sucrose sensitivity) or rpsL (S12 ribosomal protein—streptomycin sensitivity). In some embodiments, the positive selectable marker is selected from the group consisting of CmR (chloramphenicol resistance) or KanR (kanamycin resistance).

### Excision/Introduction of Double Stranded Breaks

The methods of the invention include a mechanism for excision/introduction of double stranded breaks. Suitably excision is performed to generate a linear donor nucleic acid.

In a particular embodiment, the system is the CRISPR/Cas9 system. However, other systems producing this function are also known.

For example, there have been three published papers about alternative RNA-guided endonucleases as alternates to the original Streptococcus pyogenes CRISPR/Cas9 (Ran, F. A. et al. In vivo genome editing using Staphylococcus aureus Cas9. Nature 520, 186-191 (2015); Zetsche, B. et al. Cpf1 is a single RNA-guided endonuclease of a class 2 CRISPR-Cas system. Cell 163, 759-771 (2015); Lee, C. M., Cradick, T. J. & Bao, G. The Neisseria meningitidis CRISPR-Cas9 System Enables Specific Genome Editing in Mammalian Cells. Mal. Ther. 24, 645-654 (2016).) They can all be used to guide in vivo excision in the invention. These references are expressly incorporated herein by reference specifically for the teachings of alternate systems for introduction of double stranded breaks/excisions as used herein.

### CRISPR/Cas9 Sequences

The CRISPR/Cas9 system is described in Jiang et al 2013 (Jiang, W., Cox, D., Zhang, F., Bikard, D. & Marraffini, L. A. RNA-guided editing of bacterial genomes using CRISPR-Cas systems. Nature Biotechnology 31, 233-239 (2013)).

In outline, guide RNA refers to the single fusion RNA between tracrRNA and spacerRNA. Suitably a combination of the constant tracrRNA and different spacerRNAs is used in the invention, as discussed herein. These tracrRNA spacerRNA combinations can optionally be replaced with multiple different guideRNAs.

In the art, the guide RNA only refers to the fusion of tracrRNA and spacerRNA as a single RNA, and does not mean the dual-RNA complex of tracrRNA and spacerRNA.

PAM stands for protospacer adjacent motif. This is typically a 3 nucleotide motif. A typical guide RNA is 30 nucleotides in length. The guide RNA typically comprises 27 nucleotides of target sequence as well as the 3 nucleotides of PAM sequence.

Suitably the same CRISPR setup of separate tracr RNA/spacer RNA as in Jiang et al 2013 may be used in the invention. Alternatively, a single guide RNA CRISPR setup may be used, for example as known in the art (see Le Cong et al. Multiplex genome engineering using CRISPR/Cas systems. Science 339, 819-823 (2013); Mali, P. et al. RNA-guided human genome engineering via Cas9. Science 339, 823-826 (2013)).

In order for the excision to be supported when CRISPR/Cas 9 is used, suitably the helper protein(s) capable of supporting nucleic acid excision comprise a minimum of: Cas9 (e.g. see below), and RNAseIII (e.g. rnc, accession ID EG10857 from EcoCyc), together with the relevant RNAs (spacerRNA guide, tracrRNA (see below)).

Exemplary sequences are provided below:

Cas9
(SEQ ID NO: 10)
ATGGATAAGAAATACTCAATAGGCTTAGATATCGGCACAAATAGCGTCGGATGGGCGGTGATCACTGATGA
ATATAAGGTTCCGTCTAAAAAGTTCAAGGTTCTGGGAAATACAGACCGCCACAGTATCAAAAAAAATCTT
ATAGGGGCTCTTTTATTTGACAGTGGAGAGACAGCGGAAGCGACTCGTCTCAAACGGACAGCTCGTAGAA
GGTATACACGTCGGAAGAATCGTATTTGTTATCTACAGGAGATTTTTTCAAATGAGATGGCGAAAGTAGAT
GATAGTTTCTTTCATCGACTTGAAGAGTCTTTTTTGGTGGAAGAAGACAAGAAGCATGAACGTCATCCTAT
TTTTGGAAATATAGTAGATGAAGTTGCTTATCATGAGAAATATCCAACTATCTATCATCTGCGAAAAAAAT
TGGTAGATTCTACTGATAAAGCGGATTTGCGCTTAATCTATTTGGCCTTAGCGCATATGATTAAGTTTCGT
GGTCATTTTTTGATTGAGGGAGATTTAAATCCTGATAATAGTGATGTGGACAAACTATTTATCCAGTTGGTACA
AACCTACAATCAATTATTTGAAGAAAACCCTATTAACGCAAGTGGAGTAGATGCTAAAGCGATTCTTTCTG
CACGATTGAGTAAATCAAGACGATTAGAAAATCTCATTGCTCAGCTCCCCGGTGAGAAGAAAAATGGCTTA
TTTGGGAATCTCATTGCTTTGTCATTGGGTTTGACCCCTAATTTTAAATCAAATTTTGATTTGGCAGAAGA
TGCTAAATTACAGCTTTCAAAAGATACTTACGATGATGATTTAGATAATTTATTGGCGCAAATTGGAGATC
AATATGCTGATTTGTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAACATCATCAAGACTT
GACTCTTTTAAAAGCTTTAGTTCGACAACAACTTCCAGAAAAGTATAAAGAAATCTTTTTTGATCAATCAA
AAAACGGATATGCAGGTTATATTGATGGGGGAGCTAGCCAAGAAGAATTTTATAAATTTATCAAACCAATT
TTAGAAAAAATGGATGGTACTGAGGAATTATTGGTGAAACTAAATCGTGAAGATTTGCTGCGCAAGCAACGG
ACCTTTGACAACGGCTCTATTCCCCATCAAATTCACTTGGGTGAGCTGCATGCTATTTTGAGAAGACAAGAAG
ACTTTTATCCATTTTTAAAAGACAATCGTGAGAAGATTGAAAAAATCTTGACTTTTCGAATTCCTTATTAT
GTTGGTCCATTGGCGCGTGGCAATAGTCGTTTTGCATGGATGACTCGGAAGTCTGAAGAAACAATTACCCC
ATGGAATTTTGAAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTACGGTTTATAAC
GAATTGACAAAGGTCAAATATGTTACTGAAGGAATGCGAAAACCAGCATTTCTTTCAGGTGAACAGAAGAA
AGCCATTGTTGATTTACTCTTCAAAACAAATCGAAAAGTAACCGTTAAGCAATTAAAAGAAGATTATTTCA
AAAAAATAGAATGTTTTGATAGTGTTGAAATTTCAGGAGTTGAAGATAGATTTAATGCTTCATTAGGTACC
TACCATGATTTGCTAAAAATTATTAAAGATAAAGATTTTTTGGATAATGAAGAAAATGAAGATATCTTAGA
GGATATTGTTTTAACATTGACCTTATTTGAAGATAGGGAGATGATTGAGGAAAGACTTAAAACATATGCTCA
CCTCTTTGATGATAAGGTGATGAAACAGCTTAAACGTCGCCGTTATACTGGTTGGGGACGTTTGTCTCGAAAA
TTGATTAATGGTATTAGGGATAAGCAATCTGGCAAAACAATATTAGATTTTTTGAAATCAGATGGTTTTGC
CAATCGCAATTTTATGCAGCTGATCCATGATGATAGTTTGACATTTAAAGAAGACATTCAAAAAGCACAAG
TGTCTGGACAAGGCGATAGTTTACATGAACATATTGCAAATTTAGCTGGTAGCCCTGCTATTAAAAAAGGT
ATTTTACAGACTGTAAAAGTTGTTGATGAATTGGTCAAAGTAATGGGGCGGCATAAGCCAGAAAATATCGT
TATTGAAATGGCACGTGAAAATCAGACAACTCAAAAGGGCCAGAAAAATTCGCGAGAGCGTATGAAACGA
ATCGAAGAAGGTATCAAAGAATTAGGAAGTCAGATTCTTAAAGAGCATCCTGTTGAAAATACTCAATTGCA
AAATGAAAAGCTCTATCTCTATTATCTCCAAAATGGAAGAGACATGTATGTGGACCAAGAATTAGATATTA
ATCGTTTAAGTGATTATGATGTCGATCACATTGTTCCACAAAGTTTCCTTAAAGACGATTCAATAGACAAT
AAGGTCTTAACGCGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGATAATTTAACGA
AAGCTGAACGTGGAGGTTTGAGTGAACTTGATAAAGCTGGTTTTATCAAACGCCAATTGGTTGAAACTCGC
CAAATCACTAAGCATGTGGCACAAATTTTGGATAGTCGCATGAATACTAAATACGATGAAAATGATAAACT
TATTCGAGAGGTTAAAGTGATTACCTTAAAATCTAAATTAGTTTCTGACTTCCGAAAAGATTTCCAATTCT
ATAAAGTACGTGAGATTAACAATTACCATCATGCCCATGATGCGTATCTAAATGCCGTCGTTGGAACTGCT
TTGATTAAGAAATATCCAAAACTTGAATCGGAGTTTGTCTATGGTGATTATAAAGTTTATGATGTTCGTAA
AATGATTGCTAAGTCTGAGCAAGAAATAGGCAAAGCAACCGCAAAATATTTCTTTTACTCTAATATCATGA
ACTTCTTCAAAACAGAAATTACACTTGCAAATGGAGAGATTCGCAAACGCCCTCTAATCGAAACTAATGGG
GAAACTGGAGAAATTGTCTGGGATAAAGGGCGAGATTTTGCCACAGTGCGCAAAGTATTGTCCATGCCCCA
AGTCAATATTGTCAAGAAAACAGAAGTACAGACAGGCGGATTCTCCAAGGAGTCAATTTTACCAAAAAGAA
ATTCGGACAAGCTTATTGCTCGTAAAAAAGACTGGGATCCAAAAAAATATGGTGGTTTTGATAGTCCAACGGT
AGCTTATTCAGTCCTAGTGGTTGCTAAGGTGGAAAAAGGGAAATCGAAGAAGTTAAAATCCGTTAAAGAGTT
ACTAGGGATCACAATTATGGAAAGAAGTTCCTTTGAAAAAAATCCGATTGACTTTTTAGAAGCTAAAGGAT
ATAAGGAAGTTAAAAAAGACTTAATCATTAAACTACCTAAATATAGTCTTTTTGAGTTAGAAAACGGTCGT
AAACGGATGCTGGCTAGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCTAAGCGTGTTAT
TTTAGCAGATGCCAATTTAGATAAAGTTCTTAGTGCATATAACAAACATAGAGACAAACCAATACGTGAAC
AAGCAGAAAATATTATTCATTTATTTACGTTGACGAATCTTGGAGCTCCCGCTGCTTTTAAATATTTTGAT
ACAACAATTGATCGTAAACGATATACGTCTACAAAAGAAGTTTTAGATGCCACTCTTATCCATCAATCCAT
CACTGGTCTTTATGAAACACGCATTGATTTGAGTCAGCTAGGAGGTGACTGA
tracrRNA
(SEQ ID NO: 11)
AAAAAGTTTAAATTAAATCCATAATGATTTGATGATTTCAATAATAGTTTTAATGACCTCCGAAATTAGTTT
AATATGCTTTAATTTTTCTTTTTCAAAATATCTCTTCAAAAAATATTACCCAATACTTAATAATAAATAGAT
TATAACACAAAATTCTTTTAAAAAGTAGTTTATTTTGTTATCATTCTATAGTATTAAGTATTGTTTTATGGC
TGATAAATTTCTTTGAATTTCTCCTTGATTATTTGTTATAAAAGTTATAAAATAATCTTGTTGGAACCATTC
AAAACAGCATAGCAAGTTAAAATAAGGCTAGTCCGTTATCAACTTGAAAAAGTGGCACCGAGTCGGTGCTTT
TTTTGATACTTCTATTCTACTCTGACTGCAAACCAAAAAAACAAGCGCTTTCAAAACGCTTGTTTTATCATT
TTTAGGGAAATTAATCTCTTAATCCTTTT

The actual sequences of Cas9 and tracrRNA typically remain constant through all experiments. The spacerRNA sequence changes as a function of the exact CRISPR/Cas9 cutting sites. As discussed herein the methods of the invention allow for the use of universal spacers that remain constant for iterations of the invention. For instance, the spacers may be constant for every odd-numbered iteration of the invention and a different set of spacers may be constant for every even-numbered iteration of the invention.

Suitably Cas9, tracrRNA and spacerRNA are provided together to the cell in which the excision takes place (i.e. the host cell). Suitably all three of these elements are essential for efficient excision.

In one embodiment the tracrRNA is constitutively expressed in the host cell. The Cas9 is induced together with the helper protein(s) capable of supporting nucleic acid recombination (such as lambda red alpha/beta/gamma). The spacerRNA may be provided to the host cell by transforming the cell with a small plasmid expressing spacerRNA(s). Alternatively, in a preferred embodiment, the spacerRNAs are encoded by the episomal replicon comprising the donor nucleic acid sequence. When all the three components are in the cell, the excision happens.

In another embodiment, the nucleic acid (such as DNA) sequence to express the Cas9, the tracrRNA, and the spacerRNA can be provided together to the cell, while the actual expression of (some of) the three components may be suppressed (uninduced/silent). At the appropriate time, the expression may induced, and thus inducing the excision.

In an embodiment, the tracrRNA is constantly (constitutively) expressed, the expression of Cas9 is induced, and the spacerRNA is provided last to trigger excision.

Induction of expression is well within the abilities of a skilled worker in the art. For example, the sequence of interest (such as Cas9) is placed under the control of an inducible promoter. That promoter activity is induced when desired. For example, the well-known arabinose (pAra) promoter may be used, which is induced in the presence of arabinose. Similarly, the skilled worker may choose constitutive promoters from a vast array of well-known promoters suitable for constitutive expression as desired.

As is well known in relation to operating the CRISPR system, the sequences of spacerRNAs are different for different target sites. Choosing appropriate spacerRNAs is well within the ambit of the skilled person.

TABLE 3a
Spacers used for REXER and CONEXER

| Spacers type | Target(s) | 5′ spacer sequence (5′ −> 3′) | 3′ spacer sequence (5′ −> 3′) |
|---|---|---|---|
| universal | 100k01, | GATGCAAGTGTGTCGCTGTCGACAGGCCCT | CGGCCGCCCTTTAGTGAGGGTAAGCTTCCT |
| spacers | 100k03, | (SEQ ID NO: 9) | (SEQ ID NO: 12) |
| | 100k05 | | |
| universal | 100k02, | GATGCAAGTGTGTCGCTGTCGACAGGCCCT | ATTAATTGTCAACACGTGCTCAAGCTTCCT |
| spacers | 100k04, | (SEQ ID NO: 9) | (SEQ ID NO: 13) |
| | 100k06 | | |
| HR-specific | 100k13 | CCGGATTGCGCGTAATCGTCACCATCCCCT | TTCAGCAAGGAGCTGTGAAAATGTCTCCCT |
| spacers | | (SEQ ID NO: 14) | (SEQ ID NO: 15) |
| HR-specific | 100k22 | CATTTTCAACATTGTTGCAGCTGGCAGCCT | GGATATCTGGGGCATGACATGGAAGACCCT |
| spacers | | (SEQ ID NO: 16) | (SEQ ID NO: 17) |
| HR-specific | 100k24 | TGATTGTAATGCTTAGCCATTATCTTCCCT | AACCAGGAAACAGAACCTCTGACAATGCCT |
| spacers | | (SEQ ID NO: 18) | (SEQ ID NO: 19) |
| HR-specific | 100k28 | GACGAAACCTAACAGGAAGCACATCACCCT | CCTTGTTACCTGATCAGCGTAAACACCCCT |
| spacers | | (SEQ ID NO: 20) | (SEQ ID NO: 21) |
| HR-specific | 100k37a | TGTGCCAGGCGCAGGTTGACCATGCTGCCT | CATCTTATCCAGCAACCAGGTCGCATCCCT |
| spacers | | (SEQ ID NO: 22) | (SEQ ID NO: 23) |
| pBACe3.6- | human BACs | GACATTTAGGTGACACTATAGAAGGATCCG | CGGATCCTTCTATAGTGTCACCTAAATGTC |
| backbone | (hDNA- | | |
| specific spacer | orientation) | | |

| | | | | | | | | |
|---|---|---|---|---|---|---|---|---|
| Spacers | Universal1 | Universal2 | 100k13 | 100k22 | 100k24 | 100k28 | 100k37a | Universal3 |
| reference | spacers | spacers | spacers | spacers | spacers | spacers | spacers | spacers |

TABLE 3b
Spacers used for host factor knock-out

| Spacers reference | Spacer Sequence |
|---|---|
| recA | acaatttggtaaaggctccatcatgcgcct |
| recQ | ttgatagcaaagggattttccgccaccagt |
| recN | ctggttttcttccagccagcgcagagccgc |
| ruvC | cacgcccgcatagatgagtttcagacgaga |
| sbcD | CGCGAAGCTGAACATCAGGCTTTTCTTGAC |
| mutH | acgccagagaatttaaaacgcgataaaggc |
| recB | agaggcttcaatcaggcgctcaccctgtaa |
| recO | cagcgcgcatttgtcctgcatagtcgcccg |
| rmuC | gcaaaacaacaaattacccaaagcgagcac |
| rpnB | catgccagtgatgatgagttgtttgccagt |
| sbcC | gaagtgaaaggtgaagcgtaccgtgcattc |
| uvrD | aagacgcgcgtactggtgcatcgtatcgcc |
| dinG | gtccgcaatcatctgccgctgcggcgcacg |
| helD | cccggcattgaggatcaccgcccgatcgta |
| ifhA | gaaatgtcagaatatctgtttgataagctt |
| muts | atcacccatccggtaaaacagcaggatctc |
| radA | tgtaatgaatgcggggccgattatccgcgc |
| rarA | gaagccgggcatttacattctatgatcctc |
| recC | attctggtgcaaagtaccggtatggcacag |
| recD | tgtgtcagtgaaatcggtgagctacaaaat |

The tracrRNA & spacerRNA combination can be provided separately, or can be provided as a single guide RNA, which is a fusion of the tracrRNA & spacerRNA.

It should be noted that different motifs are required for different elements of the CRISPR system. For Cas9, the PAM is NGG. In more detail, alternative implementations of the CRISPR system may be used depending on operator choice, for example implementations which lead to alternative PAMs being used. In more detail, it has been demonstrated that the Streptococcus pyogenes CRISPR/Cas9 system, which naturally recognizes NGG as PAM, can be engineered to recognize altered PAM (Kleinstiver, B. P. et al. Engineered CRISPR-Cas9 nucleases with altered PAM specificities. Nature (2015). doi:10.1038/nature14592). The three alternative RNA-guided endonuclease systems as mentioned in Ran 2015, Zetsche 2015 and Lee 2016 (see above) naturally have different PAMs.

The skilled operator will realise that if alternative components of the CRISPR system are employed in the invention then the corresponding alternate cognate PAM sequence should be used. This is well within the ambit of the skilled worker. In case any further guidance is needed, the following table shows alternative elements of the CRISPR system together with their PAM sequences.

TABLE 4

| Name | Species | PAM/Motif | Reference | PMID |
|---|---|---|---|---|
| Cas9 | Streptococcus pyogenes | NGG | 10.1126/science.1225829 | 22745249 |
| Cas9 variant | Streptococcus | NGGNG | 10.1038/nature13579 | 25079318 |
| | thermophilus A | | | |
| Cas9 variant | Streptococcus | NNAGAAW | 10.1038/nature14592 | 26098369 |
| | thermophilus B | | | |
| Cas9 variant | Staphylococcus aureus | NGRRT | 10.1038/nature14592 | 26098369 |
| | | or | | |
| | | NGRRN | | |
| Cas9 variant | Neisseria meningitidis | NNNNGATT | 10.1038/nmeth.2681 | 24076762 |
| Cas9 variant | Treponema denticola | NAAAAC | 10.1038/nmeth.2681 | 24076762 |
| Cas9 variant | Listeria innocua | NGG | 10.1038/nature13579 | 25079318 |
| Cas9 variant | Francisella novicida | NG | 10.1038/nature13579 | 25079318 |
| Cas9 variant | Lactobacillus buchneri | NAAAAN | 10.1038/nature13579 | 25079318 |
| Cas9 variant | Campylobacter jejuni | NNNNACA | 10.1038/nature13579 | 25079318 |
| Cas9 variant | Pasteurella multocida | GNNNCNNA | 10.1038/nature13579 | 25079318 |
| eSpCas9 | Streptococcus pyogenes | NGG | 10.1126/science.aad5227 | 26628643 |
| SpCas9-HF1 | Streptococcus pyogenes | NGG | 10.1038/nature16526 | 26735016 |
| Cas9-nickase | D10A & H840A of | NGG | 10.1038/nmeth.2857 | 24584192 |
| | S. pyogenes | | | |
| dCas9-FokI | Streptococcus pyogenes | NGG | 10.1038/nbt.2908 | 24770325 |
| Cpfl | Francisella novicida | TTN | 10.1016/j.cell.2015.09.038 | 26422227 |
| NgAgo | Natronobacterium | programmable | 10.1038/nbt.3547 | 27136078 |
| | gregoryi | | | |
| TtAgo | Thermus thermophilus | programmable | 10.1038/nature12971 | 24531762 |
| PfAgo | Pyrococcus furiosus | programmable | 10.1093/nar/gkv415 | 25925567 |

### Manipulation of PAM Sites for Controlled Excision

When operating the invention, the PAMs on the target sequence may be compared to the PAMs on the donor nucleic acid (e.g. sDNA) going into the target and if necessary mutated so as to avoid a double excision problem (e.g. excision accidentally including the homologous recombination sequences) if the PAM sequences match on the donor nucleic acid (e.g. sDNA) and target nucleic acid (e.g. genome DNA). This is easily done by the skilled worker in arranging the elements in the order as taught herein.

The homologous regions flanking the donor nucleic acid (e.g. synthetic DNA) on the episomal replicon are optionally further flanked by AvrII sites (CCTAGG). The AGG or CCT corresponds to the NGG PAM sequence (depending on the orientation) required by the CRISPR/Cas9 system from Streptococcus pyogenes, while the complementing CCT or AGG constitutes the last three nucleotides of the protospacer. Any substitution in the last three nucleotides of the protospacer and/or any of the G in the NGG PAM will disable CRISPR/Cas9 recognition and/or cut.

Some embodiments comprise introducing double strand breaks into the target nucleic acid into which the donor nucleic acid is to be recombined. In such embodiments, care is needed in choosing the PAM employed on the sequence resident in the nucleic acid being altered (target nucleic acid). The reason is that the sequence on the donor nucleic acid (such as DNA being introduced into the target nucleic acid) should not match the PAM on the target nucleic acid. If those do match, then the excision step of the method of the invention risks also introducing double stranded breaks in the target nucleic acid at an inappropriate location. Therefore, suitably the PAM on the target nucleic acid (such as the genome or the plasmid or the BAC into which the donor DNA is being introduced) should be compared to the PAM on the episomal replicon bearing the donor nucleic acid; if the PAM sequences are found to match then they should be mutated on the target nucleic acid being altered so as to avoid this possible problem. This is well within the ambit of the skilled reader. In more detail, in these embodiments, the two cut sites on the episomal replicon is differentiated from the corresponding end of the homologous regions on the target nucleic acid in the same way as disclosed above. The two additional cut sites on the inner side of the homologous regions on the target nucleic acid need to be identified by looking for NGG motifs, which define the boundary of the homologous regions on the target nucleic acid. The NGG PAMs of the two additional cut sites on the inner side of the homologous regions on the target nucleic acid also need to be absent on the corresponding end of the homologous regions on the episomal replicon bearing the donor nucleic acid to avoid the “double excision”. This can be very easily achieved as the sequence for insertion is naturally different from the cut sites on the target nucleic acid (such as the genome). This should be carefully arranged when the donor nucleic acid (e.g. synthetic DNA) has similar sequence to the target nucleic acid (such as wildtype genomic DNA). This is achieved in replacement by changing the corresponding NGG in the donor nucleic acid (e.g. synthetic DNA) and/or the last three nucleotide in the otherwise protospacer right next to the NGG. In this way, the inventors mark the cut sites only to the target nucleic acid (e.g. genome) positions.

In one embodiment it may be desirable to induce a cut on the target nucleic acid in order to assist in selection for recombinants. In this embodiment suitably there are 3 cuts—two on the episomal replicon to excise the donor nucleic acid and one on the target nucleic acid to assist in selection. Thus suitably said target nucleic acid comprises in order: 5′—homologous recombination sequence 1—cut site—homologous recombination sequence 2-3′.

Suitably said target nucleic acid comprises in order:
a) 5′—homologous recombination sequence 1—cut site—homologous recombination sequence 2-3′
b) 5′—homologous recombination sequence 1—positive selectable marker—homologous recombination sequence 2—3′, further comprising a cut site between said homologous recombination sequence 1 and homologous recombination sequence 2
c) 5′—homologous recombination sequence 1—negative selectable marker—homologous recombination sequence 2—3′, further comprising a cut site between said homologous recombination sequence 1 and homologous recombination sequence 2
d) 5′—homologous recombination sequence 1—positive selectable marker—negative selectable marker—homologous recombination sequence 2—3′, further comprising a cut site between said homologous recombination sequence 1 and homologous recombination sequence 2
e) 5′—homologous recombination sequence 1—negative selectable marker—positive selectable marker—homologous recombination sequence 2—3′, further comprising a cut site between said homologous recombination sequence 1 and homologous recombination sequence 2

When applying the invention in multiple rounds, the donor nucleic acid of a first round may contribute/become part of the target nucleic acid in next round. Thus, suitably the sequence of interest may comprise in order:
a) 5′—homologous recombination sequence 1—cut site—homologous recombination sequence 2-3′
b) 5′—homologous recombination sequence 1—positive selectable marker—homologous recombination sequence 2—3′, further comprising a cut site between said homologous recombination sequence 1 and homologous recombination sequence 2
c) 5′—homologous recombination sequence 1—negative selectable marker—homologous recombination sequence 2—3′, further comprising a cut site between said homologous recombination sequence 1 and homologous recombination sequence 2
d) 5′—homologous recombination sequence 1—positive selectable marker—negative selectable marker—homologous recombination sequence 2—3′, further comprising a cut site between said homologous recombination sequence 1 and homologous recombination sequence 2
e) 5′—homologous recombination sequence 1—negative selectable marker—positive selectable marker—homologous recombination sequence 2—3′, further comprising a cut site between said homologous recombination sequence 1 and homologous recombination sequence 2

Suitably the cut site on the target nucleic acid or sequence of interest is different from the excision site on the episomal replicon/donor nucleic acid.

Said cut site may be between said positive/negative selectable markers, or may be within said positive/negative selectable markers. Suitably said target nucleic acid comprises two such cut sites. Suitably said cut site is adjacent to one of said homologous recombination sequences. Suitably said two cut sites comprise a first cut site adjacent to said homologous recombination sequence 1, and a second cut site adjacent to said homologous recombination sequence 2.

### Applications

The invention involves the introduction of a sequence of interest into a target nucleic acid. “Introducing a sequence of interest”, as used herein, means that the sequence of interest is integrated into the target nucleic acid such that the resultant nucleic acid sequence comprises the sequence of interest. This may be referred to as incorporation of the sequence of interest into a target nucleic acid.

Introducing a sequence of interest into a target nucleic acid may comprise replacing a part of the target nucleic acid sequence with the sequence of interest. Thus, after a replacement, the resultant nucleic acid sequence comprises the sequence of interest and only part of the original sequence of the target nucleic acid. For example, in embodiments wherein a genome is the target nucleic acid sequence, a part of the genome sequence may be replaced by the introduced sequence of interest during an iteration of the method of the invention. The sequence of interest may replace a region within the target nucleic acid that is smaller, the same size, or larger than the sequence of interest.

Introducing a sequence of interest may comprise inserting the sequence of interest into the target nucleic acid. As used herein, “inserting” means that that the sequence of interest is introduced into a site within the target nucleic acid such that the resultant nucleic acid sequence comprises all of the original sequence of the target nucleic acid and also includes the inserted sequence of interest.

Methods of the invention may include multiple steps whereby a donor nucleic acid, for instance encoding selection markers, is introduced into the target nucleic acid and then replaced by a sequence of interest.

As an example, the methods of the invention may be used to insert a sequence of interest into a genome, or other target nucleic acid, such that all original sequences of the genome remain in addition to the newly inserted sequence of interest. In another example, the methods of the invention may be used to replace part of a genome, or other target nucleic acid, with a sequence of interest such that the overall size of the genome is unaltered. In yet other examples, the methods of the invention may be used to replace part of a genome, or other target nucleic acid, with a sequence of interest that is longer than the replaced region, such that the resultant nucleic acid comprises only part of the original genome sequence and the overall size of the genome is increased.

The invention is useful in the construction of plasmids. The invention is useful in manipulation of host genomes. The invention is useful in the construction of artificial chromosomes such as BACs.

The invention finds particular application in the making of large sized nucleic acid constructs. The invention finds particular application in the creation of high diversity libraries. In this regard, a transformation efficiency of approximately 10⁸ is achievable using current transformation techniques. However, a transformation efficiency of 10¹⁰ or beyond is extremely challenging and/or problematic. According to the present invention, a first half-library may be created and transformed into a first host cell (population of host cells). This first half-library is then transformed with nucleic acid encoding the second half-library. By using recombination according to the present invention, those two half-libraries are then combined in vivo resulting in a library having diversity of 10¹⁰, which has advantageously been obtained having only ever needing to use a transformation efficiency of 10⁵.

### Host Cell

Suitably the host cell is a prokaryotic cell. Suitably the host cell is a bacterial cell.

In one embodiment, the host cell is in vitro i.e. in the laboratory. In one embodiment, the methods of the invention are in vitro methods. In some embodiments, the methods are not practiced in vivo. Suitably the host cell is not part of a live human or animal body. Suitably the host cell is selected from one of the host cells used in the examples below.

The host cell may be any gram-negative bacterium. The host cell may be E. Coli. The host cell may be any E. coli strain (such as MG1655 or BL21), or cells derived therefrom.

MG1655 is considered as the wild type strain of E. coli. The GenBank ID of genomic sequence of this strain is U00096 (U00096.3 as of the date of filing). BL21 is widely available commercially.

The host organism such as E. coli may be chosen, or may be manipulated, in order to inhibit naturally occurring repair mechanisms to ensure the absence of, or extremely low likelihood of, double stranded repair. For example, the RecBCD system may be mutated or inhibited provided that suitable helper protein(s) capable of supporting nucleic acid recombination in the host cell are present in place of RecBCD, e.g. the lambda Red proteins described herein or other suitable recombination support proteins. For example, in one embodiment RecBCD may be inhibited because it can interfere with lambda red components and reduce the efficiency of recombination using double strand DNA with short homology regions (e.g. around 50 bp)(degraded by RecBCD system) carried out by lambda red components. However, if long homology regions (e.g. around 3-5 kb) are used, RecBCD can be an alternative as recombination support protein(s) to lambda red components as recombination support protein(s).

### Optional Additional Steps or Features

In one embodiment, the invention may involve a first recombination step carried out by conventional techniques. This has the advantage of allowing introduction into the target site of contra-selectable markers.

Optionally the invention comprises a final step of a final recombination which may be accomplished either by the methods disclosed herein or by conventional recombination. For example, this may be advantageous in removing selectable markers which have served their purpose and are no longer required for further iterations of the methods disclosed herein.

In some embodiments, the iterative methods disclosed herein begin and continue without a first conventional homologous recombination event.

In an embodiment, the excision machinery, such as CRISPR/Cas9, is employed to cut at a site intended to be replaced by recombination event, thereby creating selective pressure against the cut (and not recombined) target nucleic acid i.e. negative selection by double stranded break. In another embodiment, this negative selection by double stranded break in the target sequence is used to improve selection with a 3-double strand break embodiment (2 double strand breaks for excision of the donor nucleic acid and one double strand break between the HR1 and HR2 sequences on the target nucleic acid making 3 DS breaks/cuts in total).

In a particular embodiment, there is provided a method of introducing a sequence of interest into a target nucleic acid comprising
1) providing an E. coli host cell comprising a genome, the genome comprising a target nucleic acid;
2) delivering a BAC to the host cell by conjugative transfer,
said BAC comprising a backbone sequence and a donor nucleic acid sequence,
wherein said donor nucleic acid sequence comprises in order: 5′—homologous recombination sequence 1—sequence of interest—homologous recombination sequence 2—3′,
wherein the backbone sequence comprises a first excision site positioned adjacent to the homologous recombination sequence 1 and a second excision site positioned adjacent to the homologous recombination sequence 2, and the first excision site comprises a first protospacer and the second excision site comprises a second protospacer, and
wherein the backbone sequence encodes a first RNA molecule comprising a spacer specific for the first protospacer, and a second RNA molecule comprising a spacer specific for the second protospacer;
3) providing lambda red proteins capable of supporting nucleic acid recombination in said host cell;
4) providing a Cas9 nuclease;
5) inducing excision of said donor nucleic acid sequence by the Cas9 nuclease;
6) incubating to allow recombination between the excised donor nucleic acid and said target nucleic acid; and
7) selecting for recombinants having incorporated said donor nucleic acid into said target nucleic acid. Any of the alternatives for the features of this method that are discussed herein may be substituted for the corresponding features of this method. For instance, the BAC may be any episomal replicon, the target nucleic acid may be any target nucleic acid, the lambda red proteins may be substituted for any suitable for the purpose, etc.

As discussed herein, steps 1) to 4) may be performed in any order or simultaneously.

In another embodiment, the method is for the assembly of a nucleic acid sequence, and comprises performing steps 1) to 7) to introduce a first donor nucleic acid sequence into a first target nucleic acid in order to create a second target nucleic acid; and then performing steps 1) to 7) to introduce a second donor nucleic acid sequence into the second target nucleic acid in order to create a third target nucleic acid. This process may be iterated, wherein the product of each iteration is the target of the next iteration. A BAC comprising a first backbone sequence may be used for each odd-numbered iteration and a BAC comprising a second backbone sequence may be used for each even-numbered iteration. The first backbone sequence and the second backbone sequence may encode different spacers and may comprise different selection markers.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

### Methodology

Strains and Plasmids Used in this Study

We used a reduced-genome, streptomycin resistant E. coli (Mds42; Scarab Genomics) as starting strain for REXER and CONEXER. The same strain was used for assembly of the human CFTR gene by BASIS. The large section of the human genome was assembled a ΔrecA mutant of the same strain. All cloning procedures were performed in E. coli DH10b. We performed yeast assemblies in strain BY4741.

We used the following BACs in this study −100k13, 100k22, 100k24, 100k25, 100k26, 100k27, 100k28 and 100k37a¹⁴. Each BAC carries ˜100 kb of synthetic DNA with a defined synonymous codon compression scheme in which two serine codons (TCG and TCA) and a stop codon (TAG) are replaced through defined recoding rules (TCG to AGC, TCA to AGT, and TAG to TAA).

We used the following positive and negative selection markers in REXER and CONEXER: sacB (conferring sucrose sensitivity), cat (chloramphenicol resistance), rpsL (streptomycin sensitivity), kan^{R} (kanamycin resistance), pheST^{251A_A294G} (pheS*) (4-chloro-phenylalanine (4-CP) sensitivity), and Hyg^{R} (hygromycin resistance)¹⁴. For REXER, we used reduced-genome streptomycin resistant E. coli strains (Mds42) carrying a genomic double selection cassette at the upstream end of the integration site (locus⁰): rpsL-kan^{R} for REXER with 100k13 and 100k37a; sacB-cat for REXER with 100k22, 100k24, and 100k28. For CONEXER, we used reduced-genome streptomycin resistant E. coli (WT or ΔrecA as indicated) strains carrying a genomic double selection cassette at the upstream end of the integration site as recipient cells (locus⁰): rpsL-kan^{R} for CONEXER with 100k25 and 100k27; sacB-cat for CONEXER with 100k24, 100k26, and 100k28.

We used the helper plasmid pKW20 (Wang, K. et al. Nature 539, 59-64, doi:10.1038/nature20124 (2016)). to enable excision and recombination in REXER and CONEXER. pKW20 constitutively expresses a tracrRNA, and cas9 and lambda-red components under control of an arabinose inducible promoter. Furthermore, we created a derivative plasmid without cas9 to allow lambda-red recombination without the expression of Cas9, which was employed to modify BACs for CONEXER (see below). This was done by PCR-amplification of the rest of pKW20 followed by NEBuilder HiFi DNA Assembly.

The BACs for the assembly of the large region of the human chromosome 21 (FIG. 3C) are based on BACs from the 32k-human BAC library (BACPACK genomics). They were adapted for CONEXER using lambda-red recombination as described in construction of BACs for CONEXER.

For host gene deletions we used plasmids bearing spacer sequences and pKW20. Spacer plasmids were constructed by restriction-ligation into pMB1 plasmid backbone with ssDNA oligonucleotides encoding for guides. All spacer sequences are provided in Tables 3a and 3b.

### Construction of Spacer Array

In this study we perform genomic integration of synthetic DNA from BACs of two different designs (labelled with even and odd numbers, respectively), which requires a set of universal spacers each (Universal1 and Universal2). Spacers for CONEXER BACs adapted from the human BAC library are based on a third design (Universal3). Note that a series of BACs can be designed so one single universal spacer RNA excises both 5′ and 3′ in all BACs to simplify the method further.

All spacer RNAs for REXER are expressed from plasmid pKW3_NMB1amp_tracr_spacers (Table 15) carrying an ampicillin resistance marker, a tracrRNA, and a spacer array. We constructed each array from overlapping oligonucleotides through two rounds of PCR and prepared the backbone by restriction digestion of pKW3 with AccI and EcoRI¹⁴. We combined the backbone and each array by NEBuilder HiFi DNA Assembly prior to verification by Sanger sequencing. All spacer sequences and oligonucleotide sequences are found in Tables 5 to 15.

### Construction of BACs for CONEXER

We modified the even BACs for CONEXER by integrating an origin of transfer (oriT) sequence to enable transfer by conjugation and the universal spacer array (Universal2) on the BAC backbone (Table 5). To this end, we coupled the oriT and spacer array sequences to the selection marker amp^{R}. We amplified each sequence by PCR; the plasmid pKW3_MB1amp_tracr_Universal2 served as template for amp^{R}, pRK24 (addgene #51950) for oriT, and pKW3_MIBamp_tracr_Universal2 for the spacer array. We stitched PCR products in two sequential PCRs to create the final amp^{R}-oriT-Universal2 cassette with primers creating 50 bp homology regions to pheS* and the BAC backbone. We used the cas9-free helper plasmid pLF118 to initiate lambda-red recombination and selected for the integration of the cassette onto the BAC with ampicillin. The complete integration of the cassette was first verified by Sanger sequencing and the successfully modified BAC 100k24 was additionally verified by next-generation sequencing (NGS) to ensure integrity of the entire synthetic DNA insert. All oligonucleotide sequences are listed in Tables 5 to 14.

Odd numbered BACs can be modified in an analogous way for CONEXER. The corresponding universal spacer array, Universal1, was amplified from the pKW3_MB1amp_tracr_Universal1 plasmid described above. Corresponding oligonucleotide sequences are listed in Table 8.

The odd and even CONEXER BACs provide a simple and rapid basis for integrating synthetic DNA at any point in the E. coli genome with the CONEXER protocol. To this end, the BAC backbones may directly be amplified—using the described BACs as templates—for S. cerevisiae-mediated assembly of BACs with other synthetic DNA¹⁴;⁵; Robertson, W. E. et al. Nat Protoc 16, 2345-2380, doi:10.1038/s41596-020-00464-3 (2021).

BACs from the human library were adapted for CONEXER by integration of an oriT sequence, a universal spacer array, a universal homology region, and a double selection cassette. To this end, we cloned plasmids containing all components in the correct orientation via Gibson assembly. These plasmids served as a template for PCR, where we amplify the complete sequence to be integrated into the BACs as one linear piece of DNA. We used the cas9-free helper plasmid pLF118 to initiate lambda-red recombination and selected for the integration of the cassette onto the BAC with appropriate antibiotics (hygromycin or kanamycin depending on the type of double selection cassette used). The complete integration of the cassette was first verified by genotyping the junctions at both ends of the cassette. Successfully modified BACs were additionally verified by next-generation sequencing (NGS) to ensure integrity of the entire synthetic DNA insert.

BACs for the assembly of the CFTR gene were assembled from fragments in yeast (Robertson, W. E. et al. Nature Protocols 16, doi:10.1038/s41596-020-00464-3 (2021)). Fragments were generated via PCR amplification. CONEXER BAC 100k25 served as a template for the amplification of BAC backbone fragments (containing the origin of replication, universal homology region, oriT, and universal spacer array). Genomic DNA purified from hTERT RPE-1 cells served as a template for PCR amplification of fragments of the CFTR gene which we used for assembly.

### REXER

We performed REXER¹⁴;⁵; Robertson, W. E. et al. Nat Protoc 16, 2345-2380, doi:10.1038/s41596-020-00464-3 (2021). Starting with reduced-genome streptomycin resistant E. coli cells containing the helper plasmid pKW20_CDFtet_pAraRedCas9_tracrRNA and a genomic double selection cassette, we transformed the cell with the relevant BAC and plated on LB agar with selection for the helper plasmid (5 μg/mL tetracycline), selection for the BAC (either 18 μg/mL chloramphenicol or 50 μg/mL kanamycin), and suppression of Cas9 and lambda-red expression (2% glucose). We inoculated an isolated colony in LB medium with 5 μg/mL tetracycline and antibiotic selection for the BAC and incubated the culture overnight at 37° C. with shaking. To render the cell induced and competent, we diluted the overnight culture 1:50 in LB medium with 5 μg/mL tetracycline and antibiotic selection for the BAC. When cells reached OD₆₀₀≈0.2 (usually after 2 h), we induced expression of lambda-red and Cas9 by adding arabinose to a final concentration of 0.5% (w/v) and continued incubation for one additional hour at 37° C. with shaking. We harvested the cells and rendered them electro-competent (Fredens et al., 2019; Robertson et al., 2021).

For genomic integration of synthetic DNA by REXER, we transformed the electro-competent, induced cells with 2 g of plasmid pKW3_MB1amp_tracr_spacers encoding spacer RNAs. After 1 h of recovery in 4 mL SOB medium with shaking at 37° C., we transferred the culture to 50 mL LB medium with 5 μg/mL tetracycline, selected for spacer RNAs (100 μg/mL ampicillin), and antibiotic selection for the BAC, and continued incubation at 37° C. with shaking for 3 h. We plated the culture on LB agar with 5 μg/mL tetracycline, antibiotic selection for the BAC, and agents selecting against the negative marker on the genome as well as the negative marker on the BAC backbone (200 μg/mL streptomycin against rpsL, 7.5% sucrose sacB, and/or 2.5 mM 4-CP against pheS*). After overnight incubation at 37° C., we picked 10-11 colonies and dissolved them in 30 μL water. We assessed each clone by colony PCR for the loss of the upstream genomic double selection cassette (locus⁰) and genomic integration of the downstream double selection cassette (locus¹). We further verified the first five clones by Sanger sequencing of the colony PCR-products. All oligonucleotide sequences are provided in Tables 5 to 14.

### CONEXER

CONEXER requires preparation of a conjugation competent donor cell and a recipient cell. The donor cell carries the non-transferable conjugative plasmid pJF146 (accession number MK809154.1, Fredens et al., 2019) and the BAC with the synthetic DNA for integration, an oriT sequence and a universal spacer array. The orientation of the oriT ensures that the spacer array enters the recipient cell last to minimise the risk of partial excision by premature initiation of Cas9 cleavage in the recipient cell. The recipient cell carries a genomic double selection cassette at locus⁰, marking the upstream end of the integration site, and the helper plasmid pKW20 for inducible expression of Cas9 and lambda-red components. Odd and even numbered BACs can be alternated for replacements of adjacent genomic regions in iterative CONEXER steps, with an alternating selection strategy, essentially as described for REXER and GENESIS^{14,5}.

Here, we describe CONEXER with a donor strain carrying an even (or odd) numbered BAC with a 100 kb synthetic DNA insert with rpsL-kan^{R} (or sacB-cat) followed by pheS* (or rpsL) on the BAC backbone; and a recipient strain carrying a genomic sacB-cat (or rpsL-kan^{R}) selection cassette at locus⁰. We grew the donor strain to saturation overnight in 25 ml LB medium with selection for pJF146 (50 μg/mL apramycin) and selection for the BAC (50 μg/mL kanamycin or 20 μg/mL chloramphenicol). We grew the recipient strain to saturation overnight in 25 ml LB medium with selection for the helper plasmid (5 μg/mL tetracycline), the genomic double selection cassette (20 μg/mL chloramphenicol or 50 μg/mL kanamycin) and suppression of Cas9 and lambda-red expression (2% glucose). We harvested the cells from each culture by centrifugation and washed the pellet three times in 1 mL LB medium. After the final wash we resuspended the pellets in 800 μl LB. We mixed 160 μl of recipient with 640 μl of donor, spotted the mixture onto LB agar plates and, once spots were dried, incubated the plates at 37° C. for 1 hour. Following conjugation, we washed cells off the plate and transferred all into 250 mL pre-warmed LB medium with selection for recipient cells carrying the helper plasmid (5 μg/mL tetracycline), and the BAC (50 μg/mL kanamycin or 20 μg/mL chloramphenicol), and induced expression of Cas9 and lambda-red (0.5% L-arabinose). After 1.5 hours of incubation at 37° C. with shaking we harvested cells by centrifugation and immediately transferred all into 250 ml pre-warmed LB with 50 μg/mL kanamycin (or 20 μg/mL chloramphenicol), 5 μg/mL tetracycline, and 2% glucose to terminate recombination by suppressing expression of Cas9 and lambda-red. After another 2.5 h incubation with shaking at 37° C. we spun the culture by centrifugation and resuspended the pellet in 2 mL Milli-Q filtered water. The cell suspension was spread in serial dilutions on LB agar plates with selection for the helper plasmid (5 μg/mL tetracycline), selection for the integration of the double selection cassette at locus' (50 μg/ml kanamycin or 20 μg/mL chloramphenicol), selection for the loss of the double selection cassette at locus⁰ (7.5% sucrose or 200 μg/ml streptomycin), and selection for the loss of the BAC backbone (2.5 mM 4-CP or 200 μg/ml streptomycin [not added in addition as the selection marker on the backbone is equivalent to the one at locus⁰ in this case]). In selection plates without sucrose, we added 2% glucose to suppress Cas9 and lambda-red expression.

For experiments in ΔrecA hosts (apart from the initial screen), we grew cells for 2-8 h in 250 ml pre-warmed LB with 50 μg/mL kanamycin (or 20 μg/mL chloramphenicol), 5 μg/mL tetracycline, and 2% glucose to allow for cells who received the BAC to expand prior to the 1.5 h induction of Cas9 and lambda-red expression. This increased the number of successful recombinants from CONEXER experiments.

For the assembly of human DNA in episomes some BACs were used a pheS*-HygR double selection cassette after the human DNA insert and rpsL on the backbone. To select for the maintenance of the pheS*-HygR double selection cassette we used 200 μg/mL hygromycin. To select for the loss of the pheS*-HygR double selection cassette we added 2.5 mM 4-CP. To select for the loss of the BAC backbone we used 200 μg/ml streptomycin.

After overnight incubation at 37° C., we picked 16-24 colonies and dissolved them in 30 μL water. We assessed each clone by colony PCR for the loss of the sacB-cat cassette at locus⁰ and integration of the rpsL-kan^{R} cassette at locus¹ as described for REXER. We selected 5-16 colonies with verified genotype for whole-genome sequencing by NGS. All oligonucleotide sequences are provided in Tables 5 to 14.

### Next-Generation Sequencing (NGS) and Sequencing Data Analysis

BACS and genomic DNA (gDNA) were extracted from overnight cultures of E. coli using the QIAprep Spin Miniprep Kit and DNEasy Blood and Tissue Kit (QIAGEN), respectively. Preparation for NGS has been previously described¹⁴;⁵; Robertson, W. E. et al. Nat Protoc 16, 2345-2380, doi:10.1038/s41596-020-00464-3 (2021). For preparation of many genomes, an automated workflow was implemented with a Biomek FXp (Beckman Coulter) as follows: E. coli cultures (500 μL) were grown overnight in a 1.2 mL 96-well plate, before resuspension in ATL buffer (QIAGEN, 90 μL) and Proteinase K (10 μL) and incubation at 56° C. for 2 h. AMPure XP (Beckman Coulter, 100 μL) was added to each well and the plate was vortexed (1000 rpm, 6 min). Beads were magnetised (5 min), supernatant removed, and washed with 70% EtOH (3×400 μL), before eluting gDNA with Buffer AE (QIAGEN, 100 μL). gDNA was then diluted 1:10 in H₂O and quantified using the Qubit™ dsDNA HS assay kit (Thermofisher) adapted for a connected fluorescence plate reader (Molecular Devices SpectraMAX I3), using a calibration line and 100 μL total volume in a 96-well plate (ex/em: 502/532). This data was processed onboard and used to direct subsequent dilution of gDNA to 0.25 ng/μL. Finally, we prepared paired-end sequencing libraries with the Nextera XT DNA Library Preparation Kit (Illumina) following the manufacturer's protocol but with reduced volumes: Input gDNA (0.2-0.25 ng/μL, 2 μL), TD Buffer (3 μL), ATM (2 μL), NT Buffer (1.5 μL), indexes (1 μL), NPM (3.5 μL). Index sequences were generated from the ‘Illumina Adapter Sequences’ support document (Nextera DNA indexes, pg 16, dated June 2020), purchased from Biomers and used at 10 μM. Libraries were then purified with AMPure XP magnetic beads (Beckman Coulter) as per manufacturer's instructions (7:14 bead:reaction vol. ratio), quantified by Qubit (Thermofisher), pooled and denatured according to manufacturer's instructions.

Libraries were paired-end sequenced on a MiSeq (Illumina, reagent kit v3 (600 cycles)), an Illumina HiSeq2500 (200-cycle) or a NextSeq 2000 (Illumina, P2 reagent kit v3 (100/200 cycles)). The downstream sequencing analysis was achieved with a custom Python script as described in detail previously¹⁴; Robertson et al., 2021. To generate recoding landscapes across a target genomic region we used a custom Python script as described in detail previously¹⁴; Robertson et al., 2021). The output is the frequency of recoding at each target codon plotted across the genomic region in question.

### Host-Factor KO—Strain Generation

For gene deletion by CRISPR/Cas9-mediated cleavage and lambda-red recombineering, we adapted the procedure from Jiang et al. (2013) (Jiang, W., et al. Nat Biotechnol 31, 233-239, doi: 10.1038/nbt.2508 (2013)). We cloned spacer plasmids bearing spacer sequences by restriction-ligation into pMSP43 backbone with ssDNA oligonucleotides encoding for guides. Briefly, we phosphorylated ssDNA oligonucleotides with T4 PNK (NEB), annealed and ligated with pMSP43 backbone. We transformed the obtained plasmids into E. coli DH10b and sequence verified by Sanger sequencing. Host factor single deletions were performed in reduced-genome streptomycin resistant E. coli with a sacB-cat double selection cassette integrated at LS23 bearing helper plasmid pKW20. We grew up cultures in LB to OD₆₀₀=0.2 and then added L-arabinose (0.5%) to induce Cas9 and lambda-red.

After 1.5 h of arabinose induction cells were harvested and rendered electrocompetent by washing three times with 50 mL ice-cold 20% (w/v) glycerol in Milli-Q water. For CRISPR/Cas9-mediated cleavage, a further helper plasmid expressing the target-specific spacer sequence (conferring spectinomycin resistance) was co-electroporated with a repair ssDNA oligonucleotide introducing two stop-codons and a frameshift mutation into the target gene. The cultures were recovered after electroporation in 1 ml SOB for 1 h at 37° C. and then plated on selective LB agar plates (75 μg/mL spectinomycin, 20 μg/mL chloramphenicol, and 0.5% L-arabinose for continued Cas9 activity). The following day we picked colonies from the selective plates and amplified the targeted gene region by colony PCR. Deletions were confirmed by Sanger sequencing. Subsequently, deletion strains were cured of helper plasmids (pHFXX with specR) by repeated passaging. Curing was confirmed by phenotyping.

### Continuous Genome Synthesis

For continuous genome synthesis CONEXER 100k24 was first performed in reduced-genome streptomycin resistant E. coli ΔrecA with a sacB-cat double selection cassette at LS23. On the following day 40 clones were picked from the selection plate and grown up individually. We assessed each clone by phenotyping for the loss of the sacB-cat cassette at LS23 and integration of the rpsL-kan^{R} cassette at LS24 as described for REXER. Clones with the correct phenotype (39) were subsequently pooled in equal ratios to a total volume of 25 mL. This pool of cells served as the recipient culture for CONEXER 100k25. 96 clones were picked from the selection plate and grown up individually. Again, we assessed each clone by phenotyping for the loss of the rpsL-kan^{R} cassette at LS24 and the integration of the sacB-cat cassette at LS25. Clones with the correct phenotype (72) were subsequently pooled in equal ratios to a total volume of 25 mL. This pool of cells served as the recipient culture for CONEXER 100k26. 96 clones were picked from the selection plate and grown up individually. Again, we assessed each clone by phenotyping for the loss of the sacB-cat cassette at LS25 and the integration of the rpsL-kan^{R} cassette at LS26. Clones with the correct phenotype (53) were subsequently pooled in equal ratios to a total volume of 25 mL. This pool of cells served as the recipient culture for CONEXER 100k27. On the following day, 96 clones were picked from the selection plate and grown up individually.

Again, we assessed each clone by phenotyping for the loss of the rpsL-kan^{R} cassette at LS26 and the integration of the sacB-cat cassette at LS27. Clones with the correct phenotype (77) were subsequently pooled in equal ratios to a total volume of 25 mL. This pool of cells served as the recipient culture for CONEXER 100k28. The following day 288 clones were picked from the selection plate and grown up individually. Again, we assessed each clone by phenotyping for the loss of the sacB-cat cassette at LS27 and the integration of the rpsL-kan^{R} cassette at LS28. Out of all the clones with the correct phenotype (284) 182 were sequenced by NGS.

To calculate the expected frequency of full recoded clones in continuous genome synthesis we multiplied the experimentally determined frequency of fully recoded clones of each step of CONEXER.

TABLE 5
Oligos used to clone spacer arrays for REXER and CONEXER (pKW3_MB1amp_tracr_spacers)

| | Oligo F1 (5′ −> 3′) | Oligo F2 (5′ −> 3′) | Oligo R (5′ −> 3′) |
|---|---|---|---|
| Universal1 | GACAAATAGTGCGATTACGAAATTTTTT | GATGCAAGTGTGTCGCTGTCGA | CAGCAGGACGCACTGACCGAATTCAAC |
| spacer | AGACAAAAATAGTCTACGAGGTTTTAG | CAGGCCCTGTTTTAGAGCTATGC | TCAACAAGTCTCAGTGTGCTGAAGTTTT |
| array | AGCTATGCTGTTTTGAATGGTCCCAAA | TGTTTTGAATGGTCCCAAAACCG | GGGACCATTCAAAACAGCATAGCTCTAA |
| | ACGATGCAAGTGTGTCGCTGTCGACA | GCCGCCCTTTAGTGAGGGTAAG | AACAGGAAGCTTACCCTCACTAAAGGG |
| | GGCCCT | CTTCCT | CGGCCG |
| Universal2 | GACAAATAGTGCGATTACGAAATTTTTT | GATGCAAGTGTGTCGCTGTCGA | CAGCAGGACGCACTGACCGAATTCAAC |
| spacer | AGACAAAAATAGTCTACGAGGTTTTAG | CAGGCCCTGTTTTAGAGCTATGC | TCAACAAGTCTCAGTGTGCTGAAGTTTT |
| array | AGCTATGCTGTTTTGAATGGTCCCAAA | TGTTTTGAATGGTCCCAAAACAT | GGGACCATTCAAAACAGCATAGCTCTAA |
| | ACGATGCAAGTGTGTCGCTGTCGACA | TAATTGTCAACACGTGCTCAAGC | AACAGGAAGCTTGAGCACGTGTTGACA |
| | GGCCCT | TTCCT | ATTAAT |
| 100k13 | GACAAATAGTGCGATTACGAAATTTTTT | CCGGATTGCGCGTAATCGTCAC | CAGCAGGACGCACTGACCGAATTCAAC |
| spacer | AGACAAAAATAGTCTACGAGGTTTTAG | CATCCCCTGTTTTAGAGCTATGC | TCAACAAGTCTCAGTGTGCTGAAGTTTT |
| array | AGCTATGCTGTTTTGAATGGTCCCAAA | TGTTTTGAATGGTCCCAAAACTT | GGGACCATTCAAAACAGCATAGCTCTAA |
| | ACCCGGATTGCGCGTAATCGTCACCAT | CAGCAAGGAGCTGTGAAAATGT | AACAGGGAGACATTTTCACAGCTCCTTG |
| | CCCCT | CTCCCT | CTGAA |
| 100k22 | GACAAATAGTGCGATTACGAAATTTTTT | CATTTTCAACATTGTTGCAGCTG | CAGCAGGACGCACTGACCGAATTCAAC |
| spacer | AGACAAAAATAGTCTACGAGGTTTTAG | GCAGCCTGTTTTAGAGCTATGCT | TCAACAAGTCTCAGTGTGCTGAAGTTTT |
| array | AGCTATGCTGTTTTGAATGGTCCCAAA | GTTTTGAATGGTCCCAAAACGGA | GGGACCATTCAAAACAGCATAGCTCTAA |
| | ACCATTTTCAACATTGTTGCAGCTGGC | TATCTGGGGCATGACATGGAAG | AACAGGGTCTTCCATGTCATGCCCCAG |
| | AGCCT | ACCCT | ATATCC |
| 100k24 | GACAAATAGTGCGATTACGAAATTTTTT | TGATTGTAATGCTTAGCCATTAT | CAGCAGGACGCACTGACCGAATTCAAC |
| spacer | | | |
| array | AGACAAAAATAGTCTACGAGGTTTTAG | CTTCCCTGTTTTAGAGCTATGCT | TCAACAAGTCTCAGTGTGCTGAAGTTTT |
| | AGCTATGCTGTTTTGAATGGTCCCAAA | GTTTTGAATGGTCCCAAAACAAC | GGGACCATTCAAAACAGCATAGCTCTAA |
| | ACTGATTGTAATGCTTAGCCATTATCTT | CAGGAAACAGAACCTCTGACAAT | AACAGGCATTGTCAGAGGTTCTGTTTCC |
| | CCCT | GCCT | TGGTT |
| 100k28 | GACAAATAGTGCGATTACGAAATTTTTT | GACGAAACCTAACAGGAAGCAC | CAGCAGGACGCACTGACCGAATTCAAC |
| spacer | | | |
| array | AGACAAAAATAGTCTACGAGGTTTTAG | ATCACCCTGTTTTAGAGCTATGC | TCAACAAGTCTCAGTGTGCTGAAGTTTT |
| | AGCTATGCTGTTTTGAATGGTCCCAAA | TGTTTTGAATGGTCCCAAAACCC | GGGACCATTCAAAACAGCATAGCTCTAA |
| | ACGACGAAACCTAACAGGAAGCACAT | TTGTTACCTGATCAGCGTAAACA | AACAGGGGTGTTTACGCTGATCAGGTA |
| | CACCCT | CCCCT | ACAAGG |
| 100k37a | GACAAATAGTGCGATTACGAAATTTTTT | TGTGCCAGGCGCAGGTTGACCA | CAGCAGGACGCACTGACCGAATTCAAC |
| spacer | AGACAAAAATAGTCTACGAGGTTTTAG | TGCTGCCTGTTTTAGAGCTATGC | TCAACAAGTCTCAGTGTGCTGAAGTTTT |
| array | AGCTATGCTGTTTTGAATGGTCCCAAA | TGTTTTGAATGGTCCCAAAACCA | GGGACCATTCAAAACAGCATAGCTCTAA |
| | ACTGTGCCAGGCGCAGGTTGACCATG | TCTTATCCAGCAACCAGGTCGCA | AACAGGGATGCGACCTGGTTGCTGGAT |
| | CTGCCT | TCCCT | AAGATG |

TABLE 6
Oligos used to genotype genomic loci after REXER and CONEXER

| | Oligo F (5′ −> 3′) | Oligo R (5′ −> 3′) |
|---|---|---|
| 100k13_locus0 | GTCGCCAGACAGAATGCGAAGAATTG | TGAGACTCACAAATCCACGATGACAGG |
| 100k13_locus1 | CGCTCCACTCATTCGTAATACCGTTCG | GGCAAGCGGGAATACCAAAGAAGAATAG |
| 100k22_locus0 | GCTTTACTGGTTAGCGGATGTCAGATTG | CCCGTCAGCAGGAGAATCAGAAGTAAGAAC |
| 100k22_locus1 | GGGCAAACGGTGTGGATTAAAGACG | ATCGTCACCACAAATAACGCCACAGACTC |
| 100k24_locus0 | TGGCATCACGAGACACATCACAGAG | TACAACATGACCAGCGGATTCAAG |
| 100k24_locus1 | TTACCGCCTGGTTCTCTGACTTAACGC | TTCTTGTTCCAGTTGACCTTTCTCCCAG |
| 100k28_locus0 | CATGTGGGCGTTCAGCTTTATTCTTCG | GTGCGATAGACAAAGCAGTCCTGAG |
| 100k28_locus1 | CTTTCCACTAATGCAGGCTACCCAATCACTG | CAGAACCAATGTCTTTCACCATTCCACG |
| 100k37a_locus0 | GTGTTCCGTCCAATCTCCAACAACGATCTG | AGAGGTCAACATTACAGCCAGCAGAG |
| 100k37a_locus1 | TGCATTACGACACCACCCTGAACG | AAACGCAGCCACTGAGGCAAATG |

TABLE 7
Oligos used to adapt even numbered BACs for CONEXER

| | Oligo F (5′ −> 3′) | Oligo R (5′ −> 3′) |
|---|---|---|
| AmpR | CGCGGAACCCCTATTTGTTTATTTTTCTAAATACAT | TTACCAATGCTTAATCAGTGAGGCACCTATCTC |
| | TCAAATATGTATC | |
| OriT | GGTGCCTCACTGATTAAGCATTGGTAAAGCGCTTTT | TCATCTGTTACGCCGGCGGTAGCC |
| | CCGCTGCATAACC | |
| Universal2 | GGATCCtatttcttaataactaaaaatatggtataa | gatactgagcacatcagcaggacgcac |
| spacer array | tactcttaataaatgcagtaatac | |
| PCR stitch | GTTTAAATAAggaggacaatcCGCGGAACCCCTATT | tttttagttattaagaaataGGATCCTCATCTGTTACGCCGGC |
| Amp-oriT | TGTTTATTTTTCT | GGTAGCCG |
| PCR stitch | CATTCTTCGAAAACGATCTGCGTTTCCTCAAACAGT | ccttagctttcgctaaggatgataagcgtagcgcatcaggcaa |
| Amp-oriT- | TTAAATAAggaggacaatcCGCGG | tttagcgtttggatactgagcacatcagcaggacgcac |
| Universal2 | | |

TABLE 8
Oligos used to adapt odd numbered BACs for CONEXER

| | Oligo F (5′ −> 3′) | Oligo R (5′ −> 3′) |
|---|---|---|
| AmpR | CGCGGAACCCCTATTTGTTTATTTTTCTAAATACAT | TTACCAATGCTTAATCAGTGAGGCACCTATCTC |
| | TCAAATATGTATC | |
| OriT | GGTGCCTCACTGATTAAGCATTGGTAAAGCGCTTTT | TCATCTGTTACGCCGGCGGTAGCC |
| | CCGCTGCATAACC | |
| Universal1 | GGATCCtatttcttaataactaaaaatatggtataa | gatactgagcacatcagcaggacgcac |
| spacer array | tactcttaataaatgcagtaatac | |
| PCR stitch | GTGAAGCGTCCTAAGGCTTAACGCGGAACCCCTATT | tttttagttattaagaaataGGATCCTCATCTGTTACGCCGG |
| Amp-oriT | TGTTTATTTTTCT | CGGTAGCCG |
| PCR stitch | GTTAAAGACCGTAAGCAGGCTCGTTCCAAGTATGGC | ccttagctttcgctaaggatgataagcgtagcgcatcaggca |
| Amp-oriT- | GTGAAGCGTCCTAAGGCTTAACGC | atttagcgtttggatactgagcacatcagcaggacgcac |
| Universal1 | | |

TABLE 9
Oligos used to clone a cas9-free
derivative of pKW20

| | Oligo F (5′ −> 3′) | Oligo R (5′ −> 3′) |
|---|---|---|
| pKW20_ | AATGGCGATAGAGTATAT | TAAAATATACTCTATCGC |
| without_cas9 | TTTAGATGAAGATGCTAG | CATTGCTCCCCAAATACA |
| | AGATCCCTAGG | AAACC |

TABLE 10
Oligos used to clone spacer arrays for Host Factor Kos

| | Oligo Fw | Oligo Rv |
|---|---|---|
| recA | aacacaatttggtaaaggctccatcatgcgcctg | aaacaggcgcatgatggagcctttaccaaattgt |
| recQ | aacttgatagcaaagggattttccgccaccagtg | aaacactggtggcggaaaatccctttgctatcaa |
| recN | aacctggttttcttccagccagcgcagagccgcg | aaacgcggctctgcgctggctggaagaaaaccag |
| ruvC | aaccacgcccgcatagatgagtttcagacgagag | aaactctcgtctgaaactcatctatgcgggcgtg |
| sbcD | aaccgcgaagctgaacatcaggcttttcttgacg | aaacgtcaagaaaagcctgatgttcagcttcgcg |
| mutH | aacacgccagagaatttaaaacgcgataaaggcg | aaacgcctttatcgcgttttaaattctctggcgt |
| recB | aacagaggcttcaatcaggcgctcaccctgtaag | aaacttacagggtgagcgcctgattgaagcctct |
| recO | aaccagcgcgcatttgtcctgcatagtcgcccgg | aaaccgggcgactatgcaggacaaatgcgcgctg |
| rmuC | aacgcaaaacaacaaattacccaaagcgagcacg | aaacgtgctcgctttgggtaatttgttgttttgc |
| rpnB | aaaccaagaggtcggaataatattgccgcaggtc | aacgacctgcggcaatattattccgacctcttgg |
| sbcC | aacgaagtgaaaggtgaagcgtaccgtgcattcg | aaacgaatgcacggtacgcttcacctttcacttc |
| uvrD | aacaagacgcgcgtactggtgcatcgtatcgccg | aaacggcgatacgatgcaccagtacgcgcgtctt |
| dinG | aacgtccgcaatcatctgccgctgcggcgcacgg | aaaccgtgcgccgcagcggcagatgattgcggac |
| helD | aaccccggcattgaggatcaccgcccgatcgtag | aaactacgatcgggcggtgatcctcaatgccggg |
| ihfA | aacgaaatgtcagaatatctgtttgataagcttg | aaacaagcttatcaaacagatattctgacatttc |
| mutS | aacatcacccatccggtaaaacagcaggatctcg | aaacgagatcctgctgttttaccggatgggtgat |
| radA | aactgtaatgaatgcggggccgattatccgcgcg | aaacgcgcggataatcggccccgcattcattaca |
| rarA | aacatttcgggtcttgggggtagcggcagttacg | aaacgtaactgccgctacccccaagacccgaaat |
| recC | aacattctggtgcaaagtaccggtatggcacagg | aaacctgtgccataccggtactttgcaccagaat |
| recD | aactgtgtcagtgaaatcggtgagctacaaaatg | aaacattttgtagctcaccgatttcactgacaca |

TABLE 11
Oligos used for KO of host factor genes

| | Oligo Fw | Oligo Rv |
|---|---|---|
| recA | caatttggtaaaggctccatcatgcgcctgTAATAAGCTT | gatggtttccacatccatggaacggtcttcAAGCTTATTA |
| | gaagaccgttccatggatgtggaaaccatc | caggcgcatgatggagcctttaccaaattg |
| recQ | ctttccggccgcgattgcctcgtcgtcatgTAATAAGCTT | agggatttgatagcaaagggattttccgccAAGCTTATTA |
| | ggcggaaaatccctttgctatcaaatccct | catgacgacgaggcaatcgcggccggaaag |
| recN | ctgtgcgcccgtttttctctgaaagatacgTAATAAGCTT | ttcaagctggttttcttccagccagcgcagAAGCTTATTA |
| | ctgcgctggctggaagaaaaccagcttgaa | cgtatctttcagagaaaaacgggcgcacag |
| ruvC | ggatgcatccgcaccaaagtggatgatttaTAATAAGCTT | ttccgtcacgcccgcatagatgagtttcagAAGCTTATTA |
| | ctgaaactcatctatgcgggcgtgacggaa | taaatcatccactttggtgcggatgcatcc |
| sbcD | cgcgaagctgaacatcaggcttttcttgacTAATAAGCTT | cgcatccacctgatgggtttgtgctgtctcAAGCTTATTA |
| | gagacagcacaaacccatcaggtggatgcg | gtcaagaaaagcctgatgttcagcttcgcg |
| mutH | acgccagagaatttaaaacgcgataaaggcTAATAAGCTT | gctggcacctagccagatctccagtaacacAAGCTTATTA |
| | gtgttactggagatctggctaggtgccagc | gcctttatcgcgttttaaattctctggcgt |
| recB | TgtcgccgagacactagatcctttgcgcttgTAATAAGCT | gTgccggcagaggcttcaatcaggcgctcaccAAGCTTAT |
| | TggtgagcgcctgattgaagcctctgccggcAc | TAcaagcgcaaaggatctagtgtctcggcgacA |
| recO | cagcgcgcatttgtcctgcatagtcgcccgTAATAAGCTT | ctccgtgaagacgtccagcatcaggctggtAAGCTTATTA |
| | accagcctgatgctggacgtcttcacggag | cgggcgactatgcaggacaaatgcgcgctg |
| rmuC | gcaaaacaacaaattacccaaagcgagcacTAATAAGCTT | gcgcacttcgttattgagtaactcgcactcAAGCTTATTA |
| | gagtgcgagttactcaataacgaagtgcgc | gtgctcgctttgggtaatttgttgttttgc |
| rpnB | ataaatataacccactccctcctgcgttttAAGCTTATTA | gacctgcggcaatattattccgacctcttgTAATAAGCTT |
| | caagaggtcggaataatattgccgcaggtc | aaaacgcaggagggagtgggttatatttat |
| sbcC | gaagtgaaaggtgaagcgtaccgtgcattcTAATAAGCTT | attaccgtcgggttggttacgcgcccgattAAGCTTATTA |
| | aatcgggcgcgtaaccaacccgacggtaat | gaatgcacggtacgcttcacctttcacttc |
| uvrD | aagacgcgcgtactggtgcatcgtatcgccTAATAAGCTT | aatcgagtatggcgagcagttttccacgctAAGCTTATTA |
| | agcgtggaaaactgctcgccatactcgatt | ggcgatacgatgcaccagtacgcgcgtctt |
| dinG | gcgcttcaggaacagatccccgactttattTAATAAGCTT | ggcgacgtccgcaatcatctgccgctgcggAAGCTTATTA |
| | ccgcagcggcagatgattgcggacgtcgcc | aataaagtcggggatctgttcctgaagcgc |
| helD | acaacgcttggaaaacgtctggcacagcacTAATAAGCTT | aatcccggcattgaggatcaccgcccgatcAAGCTTATTA |
| | gatcgggcggtgatcctcaatgccgggatt | gtgctgtgccagacgttttccaagcgttgt |
| ihfA | gctgaaatgtcagaatatctgtttgataagTAATAATctt | aaccagttctttggcatcccgcttgctaagATTATTActt |
| | agcaagcgggatgccaaagaactggtt | atcaaacagatattctgacatttcagc |
| mutS | cagcagtatctcaggctgaaagcccagcatTAATAAGCTT | ataaaaatcacccatccggtaaaacagcagAAGCTTATTA |
| | ctgctgttttaccggatgggtgatttttat | atgctgggctttcagcctgagatactgctg |
| radA | tgtaatgaatgcggggccgattatccgcgcTAATAAGCTT | ggtgttccaggcatgacaggcactgcactgAAGCTTATTA |
| | cagtgcagtgcctgtcatgcctggaacacc | gcgcggataatcggccccgcattcattaca |
| rarA | atttcgggtcttgggggtagcggcagttacTAATAAGCTT | ctgatactcctgctccagcaccgccagttgAAGCTTATTA |
| | caactggcggtgctggagcaggagtatcag | gtaactgccgctacccccaagacccgaaat |
| recC | attctggtgcaaagtaccggtatggcacagtAATAAGCTT | cgcaataccaaacttttgcgacagggtcatAAGCTTATTa |
| | atgaccctgtcgcaaaagtttggtattgcg | ctgtgccataccggtactttgcaccagaat |
| recD | tgtgtcagtgaaatcggtgagctacaaaatTAATAAGCTT | cctgctgaccgcttgagaagccagcaagcaAAGCTTATTA |
| | tgcttgctggcttctcaagcggtcagcagg | attttgtagctcaccgatttcactgacaca |

TABLE 12
Oligos used to verify KO of host factor genes

| | Oligo Fw | Oligo Rv |
|---|---|---|
| recA | GTAAAACGACGGCCAGTctactgcgtatgcattgcagac | cccagtttacgtgcgtagattgg |
| recQ | GTAAAACGACGGCCAGTccctctcagagactaaaagcattgc | gatataaagcagacgaatttgcccg |
| recN | GTAAAACGACGGCCAGTaccagtttatactgtacacaataacagtaatgg | agctttgatgccacaactgataacg |
| ruvC | GTAAAACGACGGCCAGTctgatgcgtctgatcgatatgc | cggaccatatgctgcacctg |
| sbcD | GTAAAACGACGGCCAGTataggttgcgatcattaatgcgac | cgcgacgaggcaagatttgc |
| mutH | GTAAAACGACGGCCAGTtcgattaccggcaacctaaaaagc | cttcccagtcttcgcgtagc |
| recB | GTAAAACGACGGCCAGTgctgacgccgcaaaaacttgC | tcggtggtttcacgcagaca |
| recO | GTAAAACGACGGCCAGTttgaagcgcgtaaagacatgc | aaagttcagagaagcgcgtctc |
| rmuC | GTAAAACGACGGCCAGTgcaggaaatgcctttccaactg | gcgacaacaggctgttcagac |
| rpnB | GTAAAACGACGGCCAGTaagccagcagagtgaataaggataaggac | cgtcatcttaaatttgaggggtgacgg |
| sbcC | GTAAAACGACGGCCAGTaagattgatttcacccgcgagc | agatttcagtgccggttaactcctc |
| uvrD | GTAAAACGACGGCCAGTatttcccggttggcatctctg | ggttcatggctttgatcagacgc |
| dinG | GTAAAACGACGGCCAGTtcgctggtgctgtagaaatttatgat | gcagtgaatttaagatcgggaatgatc |
| helD | GTAAAACGACGGCCAGTtggtggcaaatccgccaagc | cagttgctggcgtaaaacacc |
| ihfA | GTAAAACGACGGCCAGTgcgtaaacttatttgacgtgtaccg | cagattactcgtctttgggcgaA |
| mutS | GTAAAACGACGGCCAGTcttgcttcataagcatcacgcaaaa | ggatcgccaatttgttcgcag |
| radA | GTAAAACGACGGCCAGTgattgcctaccatgccaagc | gccaatcagaatggcacttcc |
| rarA | GTAAAACGACGGCCAGTgccgcgattaacgaaatctacg | cagttaccgacgcgggataatc |
| recC | GTAAAACGACGGCCAGTagcatcagcgttacgctagtttc | gtcagtcagataatgccgcaac |
| recD | GTAAAACGACGGCCAGTggaagctgtggagcacaaac | ccgcaactttttgccagttaatttcA |

TABLE 13
Oligos used to clone pHBA010_pBBR1_CONEX-components_pH_3-prime
(template for human BAC adaptation)

| | Oligo F (5′ −> 3′) | Oligo R (5′ −> 3′) |
|---|---|---|
| pBBR1 backbone | GCTGCGGCGCCCTACGGGCTTG | GCGGCGAGGCGGCTACAGCCGATAG |
| spacer array | ACCGCCGGCGTAACAGATGAGGATCCTATTTCTTAATAACTAA | AGCCCGTAGGGCGCCGCAGCGATACTGAG |
| | AAATATGGTATAATACTC | CACATCAGCAGG |
| oriT_ampR | GGAGGACAATCCGCGGAACCC | TCATCTGTTACGCCGGCGGTAG |
| pheS_hygR_univ- | GGCTGTAGCCGCCTCGCCGCGTTGACAATTAATCATCGGCAT | GGTTCCGCGGATTGTCCTCCTTAAGCCTTA |
| HR_rpsL | AGTATATCGG | GGACGCTTCACGC |

TABLE 14
Oligos used for human BAC adaptations

| | Oligo F (5′ −> 3′) | Oligo R (5′ −> 3′) |
|---|---|---|
| RP11-410P24 | TAATAAATTTGCGGCCGCTAATACGACTCACTATA | CCACCCAACCCCCACAATATTTTAATTCAGAATTCATCAGTAT |
| adaptation | GGGAGAGGATCCGCGgatactgagcacatcagcaggacgc | AAGAATTCGTTGACAATTAATCATCGGCATAGTATATCGG |
| RP11-630H12 | TAATAAATTTGCGGCCGCTAATACGACTCACTATA | GTTTGAGACCAGTCTGGGCAACAAAGCAAGATTCCATCTCTA |
| adaptation | GGGAGAGGATCCGCGgatactgagcacatcagcaggacgc | AAGAATTCcGGCCTGGTGATGATGGC |
| RP11-599F13 | TAATAAATTTGCGGCCGCTAATACGACTCACTATA | GAGGTCCAGGGAAAGGATGTGATTGGCTCATGGGGCATATA |
| adaptation | GGGAGAGGATCCGCGgatactgagcacatcagcaggacgc | TTGTGAATTCGTTGACAATTAATCATCGGCATAGTATATCGG |
| RP11-410P24 | CAGTACTGCGATGAGTGGCAGGG | GCAGTAATACAGGGGCTTTTCAAGACTGAAG |
| genotyping | | |
| junction 1 | | |
| RP11-410P24 | GTGGTGACCAGGAATGTTCAGAGCATC | GCATAAAAGACGGCTGCTGGAGG |
| genotyping | | |
| junction 2 | | |
| RP11-630H12 | CAGTACTGCGATGAGTGGCAGGG | GCAGTAATACAGGGGCTTTTCAAGACTGAAG |
| genotyping | | |
| junction 1 | | |
| RP11-630H12 | GCATAAAAGACGGCTGCTGGAGG | CAGGAGCCCATCAAAGGTGAACATGG |
| genotyping | | |
| junction 2 | | |
| RP11-599F13 | CAGTACTGCGATGAGTGGCAGGG | GCAGTAATACAGGGGCTTTTCAAGACTGAAG |
| genotyping | | |
| junction 1 | | |
| RP11-599F13 | GTGGTGACCAGGAATGTTCAGAGCATC | CTGACCCCAAACCTAGAGTCAGCATC |
| genotyping | | |
| junction 2 | | |

TABLE 15
Plasmids used

| Plasmid | Description | Genbank # | Reference |
|---|---|---|---|
| Helper (pKW20) | Contains lambda-red recombination components and Cas9 | MN927219 | Wang et al. |
| | under arabinose inducible promoter as well as tracrRNA | | 2016 |
| pLF118 | pKW20 without Cas9 | MK809153.1 | Fredens et |
| | | | al. 2019 |
| pKW3_MB1amp_tracr | Plasmid carrying spacers for REXER experiments | none | This study |
| spacers pMSP48 | pMB1-based plasmid containing a spectinomycine | none | This study |
| | resistance (used for insertion of all spacers used | | |
| | for HF Kos) | | |
| pHBA008_pBBR1_ | CONEX components as template for human BAC adaptation, | none | This study |
| CONEX-components_ | rpsL-kanR marker cassette, pheS as backbone selection | | |
| rK_3-prime | marker, oriT and spacer array | | |
| pHBA010_pBBR1_ | CONEX components as template for human BAC adaptation, | none | This study |
| CONEX-components_ | pheS-hygR marker cassette, rpsL as backbone selection | | |
| pH_3-prime | marker, oriT and spacer array | | |

### Example 1—a Rapid and General Method to Create Custom Synthetic Genomes in Escherichia coli

### Summary

Whole genome synthesis and large-scale genome engineering promise to provide powerful approaches for understanding organism function, wholesale engineering of biosynthetic pathways, and creating organisms with functions beyond those found in nature. Simple, robust, accelerated, and scalable methods for replacing genomic DNA with synthetic DNA will make genome synthesis, and large-scale genome engineering more accessible.

Here we report an approach that simplifies and accelerates the introduction of more than 100 kb of synthetic DNA into the Escherichia coli genome. Our method accomplishes this using a rapid (one day) protocol, which may be iterated to introduce even larger synthetic DNA sequences. Crucially, the method standardizes and unifies all the necessary components such that the user only needs to clone the synthetic DNA of interest into a bacterial artificial chromosome and then implement a standard protocol.

### Results

As discussed herein, it was unclear whether a derivative of REXER that used universal spacers would work because it would result in non-homologous sequences present at the ends of the excised donor nucleic acid. It was unknown as to whether this would lead to undesired insertions or deletions.

To investigate the efficiency and fidelity of REXER with universal spacers we first designed and cloned two pairs of spacer RNAs (Universal1 and Universal2) (Table 3 and SEQ ID NOs: 24 and 25) that bind to and direct the cleavage within each of the BAC backbones we have used for E. coli genome synthesis via REXER and GENESIS (FIG. 2a); Universal1 and Universal2 target the BAC backbones used for the genome sections designated with odd and even numbers respectively (FIG. 4).

We first demonstrated that the synthetic DNA is integrated into the genome scarlessly despite the non-homologous sequences at both ends resulting from universal spacer RNA mediated Cas9 excision from the BAC. We tested REXER at multiple loci with synthetic recoded DNA 100k13, 100k22, 100k24, 100k28 and 100k37 (ref¹⁴) (FIG. 4 and Table 3). In REXER, genomic integration of the synthetic DNA is selected for by simultaneous positive and negative selection with antibiotic resistance markers positioned at the upstream end of the genomic integration site and the downstream end of the synthetic DNA insert in the BAC (FIG. 1a). A successful recombination results in the loss of the genomic selection cassette at the 5′ end of the integration site (locus⁰), and the integration of a selection cassette at the 3′ end (locus¹). We confirmed successful integration in 11/11 post-REXER clones at each of the five loci tested (FIG. 2b).

We verified the sequence around the homology regions for recombination in post-REXER clones and found that all clones had lost the 6 bp non-homologous sequence (FIG. 2c). Therefore, the mismatched sequences generated by both sets of universal spacers are efficiently and reliably removed in REXER experiments at all five loci tested. We conclude that universal spacers enable the scarless integration of large synthetic DNAs into the genome.

Next, we assessed whether using universal spacers for REXER affects recombination across the entire 100 kb genomic region that is targeted for replacement with synthetic DNA. REXER between the synthetic DNA insert from the BAC and the corresponding genomic DNA that is targeted for replacement can lead to chimeric sequences that result from recombinational crossovers⁵. These crossovers are facilitated by the high degree of homology between recoded and wildtype DNA: there is approximately 98.5% sequence identity between the synthetic DNA we used to implement synonymous codon compression throughout the E. coli genome and the corresponding natural genomic DNA sequence¹⁴. We have previously shown that the chimeras that result from REXER can be very useful for identifying and fixing sequences in the synthetic DNA that are not tolerated by the cell. The crossover frequency we observe with REXER is ideal since it is low enough to yield at least one in 8 clones in which the synthetic DNA completely replaces the corresponding genomic DNA (assuming the cell tolerates the entire synthetic DNA sequence). At the same time the crossover frequency is high enough to allow regions within a synthetic DNA sequence (including individual codon positions) that are not tolerated to be efficiently pinpointed; this is achieved through the sequencing of several post-REXER clones or a pool of post-REXER clones and analysing the frequency of recoding at each codon position^{5,14}, the frequency of recoding at each codon position can be visualised by creating compiled recoding landscapes based on the sequencing data (FIG. 2d). Codon positions that are never recoded may not tolerate recoding by the scheme being tested.

We first compared the use of universal spacer RNA and HR-specific spacer RNA for REXER with the same recoded synthetic DNA, 100k24, to replace 96.5 kb of the E. coli genome. From previous REXER experiments we know that all designed synonymous codon replacements are tolerated in this region¹⁴. Whilst the compiled landscapes are comparable, REXER initiated with universal spacer RNA resulted in twice as many clones with complete integration of the synthetic DNA, with 40% completely recoded post-REXER clones (FIG. 2d and FIG. 5). Therefore, despite the presence of short non-homologous ends at the excised synthetic DNA we do not observe a higher frequency of crossovers. Surprisingly, the overall efficiency of REXER with universal spacers is at least as good as with HR-specific spacers.

We conclude that the generation of terminal mismatches on the template for recombination does not impede the recombination and integration efficiency. We suggest that the non-homologous ends of the DNA in the BAC may be removed by exonucleases prior to recombination¹⁹, or by flap endonucleases such as EcoIX during recombination²⁰, similar to the mechanism described for FEN1 in eukaryotes^{21,22}.

REXER requires two sequential rounds of competent cell preparation and electroporation and it takes 4 days to go from cells with an appropriately marked genome to having clonal colonies on a post-REXER agar plate. To accelerate and simplify the introduction of synthetic DNA into the genome, we created BACs in which universal spacer arrays and an oriT sequence are integrated into the BAC backbone (FIG. 3 and SEQ ID NOs: 26 and 27)

Upon mixing ‘donor cells’ containing these new BACs (with a synthetic DNA insert) and a non-transferable F′ plasmid with the ‘recipient cells’ of interest, we selected for BAC transfer to the recipient via conjugative transfer. We turned on the expression of the Cas9 protein and the lambda red recombination components from the helper plasmid in the recipient, with arabinose for 1.5 h, before turning their expression off with glucose for 2.5 h; the spacers were expressed from the BAC. We selected for recipient cells in which the negative selection marker had been lost from the genome and the positive selection marker had been acquired from the BAC. Using this one day universal protocol we introduced synthetic DNA (a completely recoded fragment 24; 96 kb) in place of the corresponding genomic DNA in the recipient cell. In 19% of the clones the synthetic DNA had completely replaced the corresponding genomic sequence (FIG. 3b). We named our accelerated approach CONEXER (CONjugation coupled with programmed EXcision for Enhanced Recombination).

### Example 2—Assembly of Mb Human DNA in Episomes

The assembly of large DNA in episomes provides a foundational technology for building genomes. Entirely synthetic mycoplasma genomes have been assembled in yeast before transfer to mycoplasma. The ability to synthesize the Gbp genomes of plants and animals, with chromosomes that span the tens to hundreds of Mbps, will require technologies for assembling Mbps of DNA that can be used to replace the DNA in chromosomes in a reasonable number of steps.

Notably, the human DNA used for sequencing the essentially complete human genome was primarily captured into BACs in E. coli. The repetitive nature of much human, animal and crop genome sequence makes E. coli an attractive host for assembling DNA with which to build synthetic Gbp genomes. We hypothesized that the principles we have established for CONEXER might be extended to realize the scarless assembly and cloning, through iterative insertion, of megabases of DNA in episomes in E. coli.

We designed an assembly BAC in which to iteratively insert and assemble DNA (FIG. 6a). This BAC contains approximately 50 bp of sequence homologous to one end of the next sequence to be inserted (HR1); this is immediately followed by a positive and negative selection cassette and a universal homology region (uHR), which is complementary to the other end of the sequence to be inserted. We also designed donor BACs with the CONEXER backbone, containing universal spacers and oriT (FIG. 6a). In the donor BACs, HR1 is within one end of the next DNA sequence to be inserted into the recipient BAC; this DNA sequence is followed by a distinct positive and negative selection cassette and a universal homology region. Each step of assembly (FIG. 6a,b) proceeds by conjugation of the donor BAC to recipient cells containing the assembly BAC, Cas9-mediated excision of the sequence from HR1 to the universal homology region from the donor BAC, and lambda red mediated insertion of this sequence into the assembly BAC. Selection for loss of the negative selection markers on the assembly BAC, and gain of the positive marker from the sequence excised from the donor BAC selects for cells containing the assembly BAC with the correct insertion. Cells containing the new assembly BAC provide the input for the next step of insertion. We named our approach BAC stepwise insertion synthesis (BASIS).

We first demonstrated that assembly of the 208 Kbp human Cystic Fibrosis Transmembrane regulator gene by BASIS. We assembled the CFTR gene in three steps of BASIS with 2 donor BACS and 1 recipient episome that each contained approximately 70 Kbp fragments of the gene. We verified each intermediate step and the final assembly by next generation sequencing (FIG. 6c).

Next, we demonstrated that BASIS can be used to assemble large sections of human genomic DNA, which includes exonic, intronic and intergenic regions, into a single episome. We started with a library of human BACs used for the essentially complete sequencing of the human genome. Each of these human BACs contains approximately 100 Kbp of human DNA and there is substantial overlap between human BAC sequences.

We used one step of lambda red recombination to convert members of the human BAC library, covering a region of chromosome 21, into donor BACs for BASIS; this step introduced a positive and negative selection cassette, uHR, oriT, and universal spacers. We performed three steps of BASIS to assemble a 503 Kbp episome containing 495 Kbp of human DNA. We identified correctly assembled clones by sequencing, and used these clones as an input for the next step of BASIS, the final assembly was also verified by sequencing (FIG. 6d).

### Example 3—Crossover Minimization

In our E. coli genome synthesis each step of REXER was followed by genome sequencing to identify a single correct clone that could be used as the input for the next round of REXER. Identifying the correct intermediate clone with which to proceed was necessary because only approximately 20% of the clones from each step had replaced all 100 Kbp of genomic DNA with synthetic DNA. Thus without identifying the correct clone at each step, five steps would yield fully recoded clones with a frequency of no more than 3×10⁻⁴, and therefore tens of thousands of clones would need to be sequenced to identify a single clone with the correct sequence. Therefore, while sequencing after each step of REXER was necessary to complete the synthesis it massively slowed the synthesis and added to its cost.

We envisioned iterating CONEXER by directly using an un-sequenced pool of clones from one CONEXER as the input for the next CONEXER. In order to do this we set out to identify factors that substantially increase the fraction of clones in which the genomic DNA has been completely replaced with synthetic DNA in a single step of CONEXER.

We identified 20 factors involved in DNA repair, replication, and recombination to test for their contribution to CONEXER. We deleted each of these factors in E. coli and the performed CONEXER with 100k 24 in the resulting deletion strains. These experiments identified recA and recO as factors that increase the fraction of clones with fully synthetic sequence (FIG. 6a, FIG. 9). Deletion of recA (ΔrecA) increased the fraction of fully synthetic DNA from 20% to approximately 80% for 100k 24 (FIG. 7a). We observed similar dramatic increases across several other 100 Kbp regions, underscoring the generality of our observations (FIG. 7b).

### Example 5—Rapid and Continuous Genome Synthesis

Encouraged by the increases we observed in full replacement of genomic DNA with synthetic DNA in single steps of CONEXER, we asked whether we could directly use the output from one round of CONEXER—without identifying an individual, fully recoded clone by sequencing—as the input for the next round of CONEXER.

We first performed CONEXER, to replace the E. coli genome with synthetic, recoded DNA between LS23 and LS24, in ΔrecA E. coli containing the +2/−2 selection cassette at landing site (LS) 23 in its genome (FIG. 8a, FIG. 10). We selected for loss of the negative 7 marker from the genome (in the +2/−2 cassette at LS23) and gain of the positive marker associated with the synthetic DNA (in the +1/−1 cassette at LS24). We picked clones from the selection plate and grew them overnight; in parallel with the overnight growth we stamped clones onto selective agar to ensure they had the correct post-CONEXER phenotype. The next day clones that phenotyped correctly were pooled, and used as a direct input for the next round of CONEXER (100k25), in which the genomic DNA between LS24 and LS25 was replaced with synthetic DNA (FIG. 8a). We selected for loss of the negative marker from the genome (in the +1/−1 cassette at LS24) and gain of the positive marker associated with the synthetic DNA (in the +2/−2 cassette at LS25). We picked and pooled clones, essentially as described from the previous round of CONEXER, and used the resulting pool as the input for the next round of CONEXER (100k26). We performed five rounds of CONEXER to replace the 0.5 Mbp section of the E. coli genome between LS23 and LS28 with synthetic DNA (FIG. 8a). The entire process took ten days. Sequencing revealed that after five rounds of CONEXER ten percent of clones (19 out of 182) were fully recoded across the targeted 0.5 Mbp region of the genome (FIG. 5b). We conclude that we have developed a method for rapid and continuous genome synthesis (GCS).

### DISCUSSION

We have realized a single step, one day, universal protocol for introducing at least 100 Kbp of synthetic DNA into the E. coli genome. We have identified host factor knockouts that minimize cross overs between the host genome and synthetic DNA and enable continuous genome synthesis. We demonstrated continuous genome synthesis to build 0.5 Mbp sections of the E. coli genome, from BACs in ten days. As the methods are parallelizable it will be possible to build synthetic DNA covering the genome, in 7-8 strains, in about ten days. By combining this advance with the rapid and precise methods that have been created for compiling 0.5 Mbp synthetic recoded sections into a single strain (¹⁴; ¹²), we anticipate that our advances will reduce the timescale for synthesizing E. coli genomes from years to a few weeks. Moreover, we anticipate our approach will enable the construction of many genomes in parallel, allowing genome-level hypotheses to be tested at scale, and the creation of genome libraries for discovering new cellular function.

By extending the principles we have established for E. coli genome synthesis we have realized the scarless assembly of episomes bearing large regions of the human genome. While we have exemplified the principles through the assembly of natural sequence from human BACs (Osoegawa, K. et al. Genome Res 11, 483-496, doi:10.1101/gr.169601 (2001)), the approaches may also be used to assemble synthetic DNA fragments. Moreover, the numerous methods for multiplex editing (Wang, H. H. et al. Nature 460, 894-898, doi:10.1038/nature08187 (2009); Tong, Y., et al. Nat Commun 12, 5206, doi:10.1038/s41467-021-25541-3 (2021); Jiang, W., et al., Nat Biotechnol 31, 233-239, doi:10.1038/nbt.2508 (2013); Farzadfard, F. & Lu, T. K. Science 346, 1256272, doi:10.1126/science.1256272 (2014)) in E. coli may be combined with assembly to edit large regions of assembled human DNA much more rapidly than in human, animal or plant cells. These methods may be combined with approaches for moving large episomal DNA into animal cells (Waters, V. L. Nat Genet 29, 375-376, doi:10.1038/ng779 (2001); Litzkas, P., Jha, K. K. & Ozer, H. L. Mol Cell Biol 4, 2549-2552, doi:10.1128/mcb.4.11.2549-2552.1984 (1984)), and for iterative recombination of synthetic DNA into animal chromosomes (Martella, A., et al. ACS Synth Biol 6, 1380-1392, doi:10.1021/acssynbio.7b00016 (2017); Lee, E. C. et al. Nat Biotechnol 32, 356-363, doi:10.1038/nbt.2825 (2014); Macdonald, L. E. et al. Proc Natl Acad Sci USA 111, 5147-5152, doi:10.1073/pnas.1323896111 (2014)).

Overall, the ability to rapidly assemble large DNAs in episomes, and the development of continuous genome synthesis methods, provide the foundations for rapid and scalable genome synthesis.
References 1. Santos, C. N., Regitsky, D. D. & Yoshikuni, Y. Implementation of stable and complex biological systems through recombinase-assisted genome engineering. Nat Commun 4, 2503, doi:10.1038/ncomms3503 (2013).
2. Santos, C. N. & Yoshikuni, Y. Engineering complex biological systems in bacteria through recombinase-assisted genome engineering. Nat Protoc 9, 1320-1336, doi:10.1038/nprot.2014.084 (2014).
3. Krishnakumar, R. et al. Simultaneous non-contiguous deletions using large synthetic DNA and site-specific recombinases. Nucleic Acids Res 42, e111, doi:10.1093/nar/gku509 (2014).
4. Wang, G. et al. CRAGE enables rapid activation of biosynthetic gene clusters in undomesticated bacteria. Nat Microbiol 4, 2498-2510, doi:10.1038/s41564-019-0573-8 (2019).
5. Wang, K. et al. Defining synonymous codon compression schemes by genome recoding. Nature 539, 59-64, doi: 10.1038/nature20124 (2016).
6. Itaya, M., Tsuge, K., Koizumi, M. & Fujita, K. Combining two genomes in one cell: stable cloning of the Synechocystis PCC6803 genome in the Bacillus subtilis 168 genome. Proc Natl Acad Sci USA 102, 15971-15976, doi:10.1073/pnas.0503868102 (2005).
7. Lau, Y. H. et al. Large-scale recoding of a bacterial genome by iterative recombineering of synthetic DNA. Nucleic Acids Res 45, 6971-6980, doi:10.1093/nar/gkx415 (2017).
8. Lartigue, C. et al. Creating bacterial strains from genomes that have been cloned and engineered in yeast. Science 325, 1693-1696, doi:10.1126/science. 1173759 (2009).
9. Ostrov, N. et al. Design, synthesis, and testing toward a 57-codon genome. Science 353, 819-822, doi:10.1126/science.aaf3639 (2016).
10. Dymond, J. S. et al. Synthetic chromosome arms function in yeast and generate phenotypic diversity by design. Nature 477, 471-476, doi: 10.1038/nature10403 (2011).
11. Gibson, D. G. et al. Creation of a bacterial cell controlled by a chemically synthesized genome. Science 329, 52-56, doi: 10.1126/science.1190719 (2010).
12. Wang, K., de la Torre, D., Robertson, W. E. & Chin, J. W. Programmed chromosome fission and fusion enable precise large-scale genome rearrangement and assembly. Science 365, 922-926, doi:10.1126/science.aay0737 (2019).
13. Hutchison, C. A., 3rd et al. Design and synthesis of a minimal bacterial genome. Science 351, aad6253, doi: 10.1126/science.aad6253 (2016).
14. Fredens, J. et al. Total synthesis of Escherichia coli with a recoded genome. Nature 569, 514-518, doi:10.1038/s41586-019-1192-5 (2019).
15. de la Torre, D. & Chin, J. W. Reprogramming the genetic code. Nat Rev Genet 22, 169-184, doi:10.1038/s41576-020-00307-7 (2021).
16. Mercy, G. et al. 3D organization of synthetic and scrambled chromosomes. Science 355, doi: 10.1126/science.aaf4597 (2017).
17. Richardson, S. M. et al. Design of a synthetic yeast genome. Science 355, 1040-1044, doi: 10.1126/science.aaf4557 (2017).
18. Venetz, J. E. et al. Chemical synthesis rewriting of a bacterial genome to achieve design flexibility and biological functionality. Proc Natl Acad Sci USA 116, 8070-8079, doi:10.1073/pnas.1818259116 (2019).
19. Lovett, S. T. The DNA Damage Response. Bacterial Stress Responses, 341 2nd Edition, 205-228 (2011).
20. Anstey-Gilbert, C. S. et al. The structure of Escherichia coli ExoIX—implications for DNA binding and catalysis in flap endonucleases. Nucleic Acids Res 41, 8357-8367, doi:10.1093/nar/gkt591 (2013).
21. Liu, Y., Kao, H. I. & Bambara, R. A. Flap endonuclease 1: a central component of DNA metabolism. Annu Rev Biochem 73, 589-615, doi:10.1146/annurev.biochem.73.012803.092453 (2004).
22. Anzalone, A. V. et al. Search-and-replace genome editing without double-strand breaks or donor DNA. Nature 576, 149-157, doi:10.1038/s41586-019-1711-4 (2019).

1. A method of introducing a sequence of interest into a target nucleic acid, the method comprising
a) providing a host cell
said host cell comprising an episomal replicon,
said episomal replicon comprising a backbone sequence and a donor nucleic acid sequence,
wherein said donor nucleic acid sequence comprises in order: 5′—homologous recombination sequence 1—sequence of interest—homologous recombination sequence 2—3′,
wherein the backbone sequence comprises a first excision site positioned adjacent to homologous recombination sequence 1 and a second excision site positioned adjacent to homologous recombination sequence 2,
said host cell further comprising a target nucleic acid;
b) providing helper protein(s) capable of supporting nucleic acid recombination in said host cell;
c) providing an RNA-guided DNA endonuclease;
d) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
e) inducing excision of said donor nucleic acid sequence by the RNA-guided DNA endonuclease; and
f) incubating to allow recombination between the excised donor nucleic acid and said target nucleic acid.

2. The method according to claim 1, wherein the RNA-guided DNA endonuclease is a CRISPR-Cas nuclease, the first RNA molecule comprises a spacer specific for the first excision site, and the second RNA molecule comprises a spacer specific for the second excision site.

3. The method according to claim 2, wherein the CRISPR-Cas nuclease is Cas9.

4. The method according to any one of claims 1 to 3, wherein the first RNA molecule and/or the second RNA molecule are encoded by the episomal replicon.

5. The method according to any one of claims 1 to 4, wherein each terminus of the excised nucleic acid comprises nucleic acid sequence derived from the backbone sequence.

6. The method according to claim 5, wherein the excised donor nucleic acid comprises 6 or fewer base pairs of nucleic acid sequence derived from the backbone sequence at each terminus.

7. The method according to any one of claims 1 to 6, wherein the episomal replicon is a bacterial artificial chromosome.

8. The method according to any one of claims 1 to 7, wherein the episomal replicon is delivered to the host cell by conjugative transfer.

9. The method according to any one of claims 1 to 8, wherein the target nucleic acid is the genome of the host cell.

10. The method according to any one of claims 1 to 9, wherein the host cell is a prokaryotic cell.

11. The method according to any one of claims 1 to 10, wherein the prokaryotic cell is Escherichia coli.

12. A method of assembling a nucleic acid sequence, the method comprising:
(i) performing the steps of any one of claims 1 to 11 to introduce a first donor nucleic acid sequence into a first target nucleic acid in order to create a second target nucleic acid; and
(ii) performing the steps of any one of claims 1 to 11 to introduce a second donor nucleic acid sequence into the second target nucleic acid in order to create a third target nucleic acid.

13. The method of claim 12, wherein part (i) and part (ii) are iterated.

14. The method of claim 13, wherein
the sequence of the first RNA molecule for part (i) is the same for each iteration and/or the sequence of the second RNA molecule for part (i) is the same for each iteration; and
the sequence of the first RNA molecule for part (ii) is the same for each iteration and/or the sequence of the second RNA molecule for part (ii) is the same for each iteration.

15. The method of any one of claims 12 to 14, further comprising:
(iii) performing the steps of any one of claims 1 to 11 to introduce a third donor nucleic acid sequence into the third target nucleic acid in order to create a fourth target nucleic acid;
iterating parts (i), (ii), and (iii), and wherein
the sequence of the first RNA molecule for part (iii) is the same for each iteration and/or
the sequence of the second RNA molecule for part (iii) is the same for each iteration.

16. The method of any one of claims 12 to 15, wherein part (i) comprises the use of a donor-nucleic-acid-sequence-encoding episomal replicon comprising a first backbone sequence, and part (ii) comprises the use of a donor-nucleic-acid-sequence-encoding episomal replicon comprising a second backbone sequence, wherein
the first backbone sequence comprises a first marker or set of markers, encodes the first RNA molecule specific for the first excision site within said first backbone sequence, and encodes the second RNA molecule specific for the second excision site within said first backbone sequence; and
the second backbone sequence comprises a second marker or set of markers, encodes the first RNA molecule specific for the first excision site within said second backbone sequence, and encodes the second RNA molecule specific for the second excision sites within said second backbone sequence; wherein
the first marker or set of markers is different from the second marker or set of markers.

17. A method for constructing an episomal replicon comprising the steps of:
a) providing a donor episomal replicon, said replicon comprising:
a backbone, said backbone comprising universal spacer sequences, a first homology region HRn which is specific for an integration step n, and a second, universal, homology region uHR, a first excision site positioned adjacent to HRn and a second excision site positioned adjacent to uHR;
a donor nucleic acid DNAn;
a double selection cassette, comprising positive and negative selection markers;
b) providing a host cell comprising an assembly episomal replicon comprising a double selection cassette comprising positive and negative selection markers, flanked by HRn and uHR, the double selection cassette in the assembly replicon comprising different markers to the selection cassette in the donor replicon;
c) providing helper protein(s) capable of supporting nucleic acid recombination in said host cell;
c) providing an RNA-guided DNA endonuclease;
d) providing a first RNA molecule comprising a sequence specific for the first excision site and a second RNA molecule comprising a sequence specific for the second excision site, wherein the first and the second RNA molecules contribute to directing the RNA-guided DNA endonuclease during excision;
e) inducing excision of said donor nucleic acid sequence DNAn by the RNA-guided DNA endonuclease in the host cell; and
f) incubating to allow recombination between the excised donor nucleic acid and said assembly replicon to form a second assembly replicon, which comprises the nucleic acid DNAn.

18. The method according to claim 17, wherein the RNA-guided DNA endonuclease is a CRISPR-Cas nuclease, the first RNA molecule comprises a spacer specific for the first excision site, and the second RNA molecule comprises a spacer specific for the second excision site.

19. The method according to claim 18, wherein the CRISPR-Cas nuclease is Cas9.

20. The method according to any one of claims 17 to 19, wherein the first RNA molecule and/or the second RNA molecule are encoded by the donor episomal replicon.

21. The method according to any one of claims 17 to 20, wherein each terminus of the excised nucleic acid comprises nucleic acid sequence derived from the backbone sequence.

22. The method according to claim 21, wherein the excised donor nucleic acid comprises 6 or fewer base pairs of nucleic acid sequence derived from the backbone sequence at each terminus.

23. The method according to any one of claims 17 to 22, wherein the episomal replicon is a bacterial artificial chromosome.

24. The method according to any one of claims 17 to 23, wherein the episomal replicon is delivered to the host cell by conjugative transfer.

25. The method according to claim 24, wherein the episomal replicon is comprised in a donor host cell, and the assembly replicon is comprised in a recipient host cell; the donor replicon is transferred to the recipient host cell by conjugative transfer; and the donor host cell comprises a non-transferrable F′ plasmid.

26. The method according to any one of claims 17 to 25, wherein the host cell is a prokaryotic cell.

27. The method according to any one of claims 17 to 26, wherein the prokaryotic cell is Escherichia coli.

28. The method of any one of claims 17 to 27, wherein the donor nucleic acid DNAn comprises a homology region HRn+1, and the method further comprises the steps of introducing into the host cell a further donor episomal replicon comprising a second donor nucleic acid DNAn+1, inducing excision of said donor nucleic acid sequence DNAn+1 by the RNA-guided DNA endonuclease in the host cell; and incubating to allow recombination between the excised donor nucleic acid DNAn+1 and said second assembly replicon to form a third assembly replicon, which comprises the nucleic acid DNAn and nucleic acid DNAn+1.

29. The method of claim 28, iteratively repeated.

30. A method according to any one of claims 12 to 16, wherein the episomal replicon of the steps of claims 1 to 11 is constructed according to any one of claims 17 to 29.

31. The method according to any one of claims 1 to 30, wherein the host cell is lacking competent recA and/or recO.

32. The method according to claim 31, wherein the host cell lacks recA (ΔrecA).
